(19) United States
(12) Patent Application Publication
(10) Pub. No.: US 2022/0257569 A1
(43) Pub. Date: Aug. 18, 2022

(54) INDOLE-OXADIAZOLE COMPOUNDS AND THEIR THERAPEUTIC USE

(71) Applicants: THE GOVERNING COUNCIL OF THE UNIVERSITY OF TORONTO, Toronto, Ontario (CA); THE UNIVERSITY COURT OF THE UNIVERSITY OF ABERDEEN, Aberdeen Aberdeenshire (GB)

(72) Inventors: Iain Robert GREIG, Aberdeen (GB); Ruth ROSS, Toronto, Ontario (CA); Amy RAMSEY, Toronto, Ontario (CA); Catherine MIELNIK, Oakville, Ontario (CA); Laurent Alain Claude TREMBLEAU, Aberdeen (GB); Mostafa Hamed ABDELRAHMAN, Aberdeen (GB)

(73) Assignees: THE GOVERNING COUNCIL OF THE UNIVERSITY OF TORONTO, Toronto, Ontario (CA); THE UNIVERSITY COURT OF THE UNIVERSITY OF ABERDEEN, Aberdeen Aberdeenshire (GB)

(21) Appl. No.: 17/274,933

(22) PCT Filed: Sep. 12, 2019
(86) PCT No.: PCT/CA2019/051294

§ 371 Date: Mar. 10, 2021

Related U.S. Application Data
(60) Provisional application No. 62/730,296, filed on Sep. 12, 2018.

(51) Int. Cl. A61K 31/4245

(52) U.S. Cl. CPC A61K 31/4245

(57) ABSTRACT

The present application pertains to methods of using indole-oxadiazole compounds of Formula I to modulate cannabinoid receptor activity: I In particular diseases, disorders or conditions that benefit from modulating cannabinoid receptor activity, such as non-alcoholic steatohepatitis (NASH), non-alcoholic fatty liver disease (NAFLD), schizophrenia, bipolar disorder, psychosis, metabolic syndrome, type-2 diabetes, dyslipidaemia, obesity, eating disorders, cardiovascular diseases and disorders, and other conditions as described herein, may be treated. Also included in the present application are certain novel compounds of Formula Ia and pharmaceutical compositions comprising these compounds.

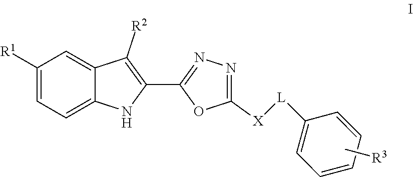

### RELATED APPLICATIONS

The present application claims the benefit of priority of co-pending U.S. provisional patent application No. 62/730,296 filed on Sep. 12, 2018, the contents of which are incorporated herein by reference in their entirety.

### FIELD

The present application relates to a method of modulating cannabinoid receptor activity using compounds of Formula I. The present application also relates to the treatment of diseases, disorders or conditions treatable by modulating cannabinoid receptor activity such as mental disorders and liver disorders. The present application also relates to indole-oxadiazole compounds of Formula Ia, to processes for their preparation and to compositions comprising them.

### BACKGROUND

The endocannabinoid system encompasses a family of endogenous eicosanoid ligands, known as “endocannabinoids”. Prominent examples include arachidonoylethanolamide (anandamide) and 2-arachidonoyl glycerol (2-AG), both of which are synthesized on demand and are rapidly hydrolyzed by the enzymes fatty acid amide hydrolase (FAAH) and monoacyl glycerol lipase (MAG lipase) respectively (see, e.g., Di Marzo, 2004). Levels of the endocannabinoids are altered in certain disease states, where they have an autoprotective role (see, e.g., Pertwee, 2005). Mammalian tissues express at least two types of cannabinoid receptor, CB1 and CB2, both G protein coupled receptors (GPCR).

Cannabinoid receptors have been shown to play an important role in many areas of human physiology and are treatments or potential treatments for a number of human medical conditions. Cannabinoid receptor agonists are already in use (e.g., Marinol®, Solvay; Nabilone®, Eli Lilly; Sativex®, GW Pharmaceuticals) as treatments for chemotherapy-induced nausea; for the control of pain and the treatment of spasticity in patients with multiple sclerosis; and as appetite enhancers for patients with HIV/AIDS or undergoing chemotherapy.

Other studies have demonstrated a role in inflammation for both the CB1 and CB2 receptors and the ability of drugs which antagonize these receptors to be used as anti-inflammatory agents in the treatment of a number of disorders, including rheumatoid arthritis, psoriasis and inflammatory bowel disease (see, e.g., Croci et al., 2003).

More recently there has been intense interest in the therapeutic properties of drugs which act as antagonists at CB1. These include SR141716A (Acomplia®, Sanofi-Aventis) and taranabant (Merck) for which clinical trials have demonstrated efficacy in the facilitation of weight loss, treatment of type-2 diabetes and cessation of smoking. The inventors have previously shown that similar compounds are able to prevent bone loss and therefore may be used in the treatment of disorders involving excessive or inappropriate bone loss, including osteoporosis, Paget's disease of bone, and bone cancers (see, e.g., Greig et al., 2004; Idris et al., 2005).

A number of drugs reached late stage clinical trials and/or were marketed. However, rejection by the FDA and eventual withdrawal in Europe of the first such drug to reach the market, rimonabant, effectively terminated this approach, in spite of the apparent therapeutic benefit. The primary problems were found to be caused by down-regulation of the receptor (due to the property of inverse agonism) leading to a reduction in efficacy and reversal of weight loss on cessation of treatment, and by CNS effects, which included suicidal thoughts, nausea and depression.

There is ample evidence that the levels of endocannabinoids are increased in both physiological and pathophysiological situations; the increased endocannabinoid levels may be exacerbating an illness or they may be having an autoprotective action (see, e.g., Pertwee, 2005).

It has long been suggested that the toxicological issues associated with cannabinoid antagonism could be avoided if a drug only tuned down excessive receptor activation, rather than turned it off completely. This effect can be achieved by the use of allosteric modulators and the therapeutic potential of cannabinoid receptor modulation can be harnessed without the associated side effects of global activation or inhibition of the receptor (see Di Marzo, 2008).

It is now acknowledged that numerous GPCRs contain allosteric binding sites for endogenous and/or synthetic ligands, which are discrete from the agonist-binding site, which is known as the orthosteric site (the normal binding site for the endogenous ligand) (see e.g., Christopoulos et al., 2002). The binding of an allosteric modulator delivers a conformational change which impacts the affinity and/or efficacy of orthosteric ligands, thereby influencing the behaviour of a receptor and tuning the pre-existing actions of the endogenous ligands (see, e.g., May et al., 2003). The action may increase or decrease the affinity of the endogenous ligand and its association or dissociation rate constant, and may either increase or decrease the intracellular activity caused by the binding of the endogenous ligand. Thus, allosteric modulators may be defined as positive or negative according to whether they enhance or inhibit the transmission of activity caused by the endogenous ligand binding.

Allosteric modulation has a number of advantages, including more subtle modulation or resetting of receptor activity than seen with intervention at the orthosteric site. As an allosteric modulator can only act in the presence of the natural ligand, it allows for drug therapy that more effectively maintains normal receptor function. Thus, allosteric modulation may be less likely to cause side-effects, as the pharmacological effects more closely model normal physiology. (See, e.g., Conn et al., 2009; Gao et al., 2006).

The endogenous ligand is generally synthesized and released on demand at the site in which the action is required; rapid breakdown ensures that the effects on distant receptors sites in other tissue types is kept to a minimum. Likewise, in disease states, receptor over-activation or under-activation may be restricted to particular tissues and it is desirable only to modulate at these sites; altering receptor activity at other sites may lead in turn to undesirable harmful effects. Synthetic ligands tend to be delivered systemically and thus affect a variety of tissue types, but cannot usually be designed to have the same binding characteristics as the endogenous ligand, due to the requirements for metabolic and chemical stability. Thus, tuning of the signal from the endogenous ligand also has the advantage of introducing tissue selectivity and thus affecting only the tissues affected by the disease state, giving a more effective therapy. With less evolutionary pressure and lower sequence conservation between receptor sub-types, the allosteric sites of many receptors offer greater opportunities for selectivity, whereas the orthosteric sites of many receptors can be too similar to allow a drug to distinguish between them (as they often must bind the same endogenous ligand, for example, a neurotransmitter). General reviews on the growing importance and promise of allosteric modulation can be found in, e.g., Conn et al., 2009. Reviews regarding the potential of cannabinoids can be found in, e.g., Ross, 2007.

Allosteric modulation may also offer an approach to targeting the activities of orthosteric receptors which are regarded as “un-druggable”, in that the physiochemical characteristics of the binding site (the mixture of hydrophobic and polar groups lining the pocket) may be incompatible with the physicochemical attributes required of a drug with sufficient solubility, membrane permeability and metabolic stability to reach the site of action. In these cases, an allosteric site may be found which has better compatibility with drug-like substances.

Furthermore, allosteric modulation may offer an approach to targeting diseases in which receptor number has been reduced (e.g., Parkinson's disease, in which the number of dopaminergic neurons is diminished).

Recent reviews highlight the emerging lucrative drug discovery potential of developing positive and negative allosteric modulators of GPCRs (see, e.g., Wang et al, 2009). Similarly, the targeting of drugs to allosteric sites on the cannabinoid CB1 receptor may offer reduced side-effect profile; greater receptor-subtype selectivity; reduced drug-induced alterations in receptor coupling mechanisms (see, e.g., Ross, 2007; Dopart et al., 2018). In particular, allosteric modulators may be beneficial in the treatment of multi-factorial syndromes, such as metabolic disorders (see, e.g., Wang et al, 2009).

In 2005, the first evidence was published indicating that the cannabinoid CB1 receptor contains an allosteric binding site and compounds were identified that were unexpectedly allosteric enhancers of agonist binding affinity, but that functioned as allosteric inhibitors of agonist activity efficacy (see, e.g., Price et al., 2005). Accordingly, allosteric inhibitors may have the potential to treat liver fibrosis, fatty liver disease, schizophrenia, bipolar disorder, metabolic syndrome (obesity, type-2 diabetes and associated conditions) and other conditions in which excessive activation of the endocannabinoid system has been implicated. They could also be used to treat Cannabis use disorder; Cannabis-induced hyperemesis and drug addiction.

Whilst a number of ligands that are selective for the cannabinoid CB1 and CB2 receptor have been developed to date, issues of receptor subtype selectivity remain. A number of well-established cannabinoid receptor agonists and antagonists have affinity for an orphan receptor GPR55 and the TRPV1 channel (see, e.g., Ryberg et al., 2007; Ross, 2003). Targeting the allosteric site on the CB1 receptor may be a key strategy for generation of highly subtype-specific compounds.

Cannabinoid receptor allosteric modulators may offer the opportunity to target specific downstream activity pathways thus giving a more targeted therapeutic outcome with reduced side effects. Cannabinoid receptor allosteric modulators may also display ligand selectivity whereby they differentially modulate the effects of specific agonists e.g. 2AG, anandamide or THC (Baillie et al., 2013).

A major hypothesis for the pathophysiology of mania (in, for example, schizophrenia (SCZ) and bipolar disorder (BD)) is the dopamine dysfunction hypothesis, developed in response to the observation that drugs which block dopamine receptors are effective in treating positive symptoms in SCZ and mania in BD (Purves-Tyson et al., 2017). Therefore, potential targets for the development of novel therapeutics may require testing in complex settings of dopamine dysregulation. In addition to dopamine, glutamate dysfunction is implicated in these disorders; phencyclidine, a non-competitive antagonist of NMDA receptors, produces schizophrenic symptoms. Ketamine and MK-801, two additional non-competitive antagonists of NMDARs, also produce schizophrenic symptoms. NMDARs are altered in SCZ, BD and depression. NMDARs appear to play a central role in onset and establishment of these mental illnesses; NMDAR hypofunction promotes increased function of mostly D2 receptors (in the striatal and prefrontal regions of schizophrenic patients; whereas in depressives NMDAR hyper-function restricts primarily serotonin activity in the prefrontal cortex.

Endocannabinoids are released ‘on demand’ and alter the release of various inhibitory and excitatory neurotransmitters. CB1 receptors are expressed in different neuronal types, including GABAergic, glutamatergic, serotonergic and dopaminergic neurons. Expression levels of CB1 receptors can significantly differ in various brain regions and neuronal cell types. The expression profile underpins the complex nature of the function of the endocannabinoid system that explains the multimodal effects of certain cannabinoid drugs and the opposing effects in different illnesses. The complex dysregulation and circuit-based mechanisms for brain region-dependent alterations in dopaminergic activity in psychiatry may be more amenable to drug targets that indirectly-modulate dopaminergic and glutamatergic activity, via direct approaches such as D2 antagonists. As such, the endocannabinoid system acts as a crucial filter that integrates activity that controls dopamine neuron activity. A recent review (Covey et al., 2017) highlights that many disease states and behaviours that have be defined as ‘dopamine dependent’ are now understood to be interactions between the dopamine and endocannabinoid system.

Notably, a recent study (Arias et al., 2017) showed that three quarters of Cannabis addicts had a dual diagnosis, mainly associated with psychosis, BD, and agoraphobia. Cannabis is known to exacerbate psychotic illness; initiating earlier onset of a more severe illness. Evidence suggests an association between Cannabis use and relative risk of developing SCZ; as either an independent or an environmental risk to vulnerable individuals (French et al., 2015).

It has also become clear over recent decades that lifestyle-related intervention is no longer sufficient for the control of obesity, a modern plague associated with many cardiovascular complications. The possibility of pharmacological assistance is being widely investigated, but as noted earlier, safety concerns and poor efficacy have severely limited the number of drugs to reach the market.

Alongside other health risks, obesity is associated with insulin resistance and impaired glucose tolerance; the endocannabinoid system is a powerful regulator of the overactive systemic metabolism seen in type-2 diabetes.

Cannabinoid antagonists have been proposed as useful therapeutic agents in this field, but again the lack of a safe agent has thus far prevented clinical exploitation. Evidence suggests that the activation of CB1 receptors in these peripheral tissues promotes lipogenesis, lipid storage, insulin secretion, glucagon secretion and adiponectin modulation (see, e.g., Cota et al. 2003; Osei-Hyiaman et al., 2005; Bermudez-Silva et al., 2008). CB1 receptors and endocannabinoids are present in peripheral tissues involved in metabolic dysfunction associated with obesity, including adipose tissue, liver, skeletal muscle and pancreas, and there is evidence for the upregulation of the endocannabinoid system in these tissues in experimental and human obesity (see, e.g., Kunos et al., 2009). Furthermore, a peripherally-restricted CB1 receptor antagonist does not affect behavioural responses in mice with genetic or diet-induced obesity, but it does cause weight-independent improvements in glucose homeostasis, fatty liver, and plasma lipid profile (see, e.g., Tam et al., 2010). These findings confirm a prominent role for peripheral CB1 receptors on the modulation of metabolism (see, e.g., Son et al., 2010).

The impact of non-alcoholic fatty liver disease (NAFLD) is a growing concern worldwide due to the increasing prevalence of obesity, metabolic syndrome, insulin resistance and Type 2 diabetes. NAFLD encompasses a spectrum of pathological changes in the liver ranging from hepatic steatosis or NAFLD to non-alcoholic steatohepatitis (NASH), which is characterized by hepatocyte ballooning and necroinflammatory steatosis with or without fibrosis.

Economically, the escalation in the prevalence of NAFLD and associated cardiovascular- and liver-related morbidity and mortality will result in an ever-increasing burden to the healthcare system. While prevention and lifestyle modification such as exercise and caloric restriction would be the first line of therapy to combat obesity and NAFLD, modifying the behavior and motivation of people before and after the development of symptoms precludes the effectiveness of lifestyle behavioral modifications and warrants the development of alternative treatment strategies such as pharmacological intervention. Existing pharmacological treatments proposed for the treatment of NAFLD include the use of thiazolidinediones (TZDs), which improve insulin sensitivity and glucose tolerance while promoting hepatic fatty acid oxidation and decreasing hepatic lipogenesis (Dietrich et al., 2014). However, long-term treatment with TZDs is highly discouraged due to complications of increased fracture risk, congestive heart failure and stroke. One system that holds promise as a potential therapeutic target for the amelioration of NAFLD is the endocannabinoid system (ECS).

Endocannabinoids are endogenous lipid ligands that bind to specific G protein-coupled receptors, which include the CB1, CB2 and GPR55 receptors, to mediate their physiological effects including the regulation of appetite and energy homeostasis. The endocannabinoids arachidonoylethanolamide (anadamide, AEA) and 2-archidonoylglycerol (2-AG) are fatty acid derivatives generated from membrane phospholipid precursors to act in a localized manner It is well known that endocannabinoids can regulate energy balance by modulating the hypothalamic regulation of food intake (Quarta et al., 2011). More importantly, increasing evidence has demonstrated that the endocannabinoid system is involved in the regulation of energy metabolism peripherally, where the local production of lipid mediators may affect activity in adipose tissue and liver. Dysregulation of the endocannabinoid system in obesity has been reviewed extensively (Vettor et al., 2009; Martins, et al., 2014; Simon et al., 2017). Hyperactivity of the endocannabinoid system is also evident in animal models of diet-induced obesity (DIO) with evidence of higher levels of endocannabinoids and an upregulation of peripheral CB1 receptors (Kunos et al., 2008, Purohit et al., 2010). Hepatic CB1 receptors can stimulate de novo lipogenesis and inhibit fatty acid oxidation, which leads to an overall accumulation of lipids within hepatocytes and a dysregulation in energy balance.

The involvement of the endocannabinoid system in obesity is supported by human genetic and genetically-modified knockout mouse studies (Tourino et al., 2010, Cota et al., 2003, Ravinet et al., 2004, Osei-Hyiaman et al., 2008, Jeong et al., 2008). Collectively, these studies support a role of the endocannabinoid system in metabolic energy balance dysfunction that can lead to NAFLD.

Initial studies using rimonabant, a CB1 receptor antagonist introduced as an anti-obesity drug were promising (Van Gaal et al., 2005; Scheen et al., 2006). However, the increased propensity for adverse psychiatric side effects, including an exacerbation in anxiety and suicide, led to the discontinuation of this drug. Studies in mice have also supported a role for CB1 receptor blockade by rimonabant in attenuating obesity and hepatic steatosis. Taken together, these data suggest that use of either a centrally-acting CB1 receptor negative allosteric modulator or a peripherally-acting negative allosteric modulator (allosteric inhibitor) would be expected to be useful in therapy for, e.g., schizophrenia, bipolar disorder, and NASH/NAFLD, but would be expected to lack the side-effects seen with CB1 receptor antagonists that target the orthosteric site.

A small number of 2-(1H-indol-2-yl)-1,3,4-oxadiazoles (“indole-oxadiazoles”) have been described.

Hiremath et al., 1989 describes 2-(1H-indol-2-yl)-1,3,4-oxadiazoles compounds, for which biological activity was not reported.

Monge et al., 1992 describe 2-(1H-indol-2-yl)-1,3,4-oxadiazole compounds, for which inhibition of platelet aggregation was reported.

Hurst et al., 2009, describes a 2-(1H-indol-2-yl)-1,3,4-oxadiazoles compound for which modulation of the nicotinic acetylcholine receptor is reported.

Peters et al., 2004 describe a 2-(1H-indol-2-yl)-1,3,4-oxadiazole compound for which modulation of the nicotinic acetylcholine receptor and modulators of the monoamine receptors and transporters is reported.

El Ashry et al., 2013 describes a 2-(1H-indol-2-yl)-1,3,4-oxadiazole compound, for which antibacterial and antifungal activity is reported.

Vasu et al., 2014 describes 2-(1H-indol-2-yl)-1,3,4-oxadiazole compounds for which immunomodulatory properties are reported.

Wang et al., 2016 describes a 2-(1H-indol-2-yl)-1,3,4-oxadiazole compound, for which no biological properties are reported.

### SUMMARY OF THE APPLICATION

The Applicant has found that certain 2-(1H-indol-2-yl)-1,3,4-oxadiazoles (“indole-oxadiazoles”) are capable of modulating cannabinoid receptor activity.

Accordingly, the present application includes a method for modulating cannabinoid receptor activity in a cell comprising administering to the cell an effective amount of one or more compounds of Formula I or a pharmaceutically acceptable salt and/or solvate thereof:

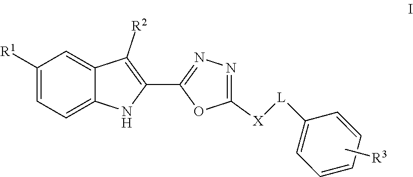

wherein:
R¹ is H, Br, Cl, F, I, C₁₋₆alkyl, SC₁₋₆alkyl or OC₁₋₆alkyl;
R² is H or C₁₋₆alkyl;

### L is C₀₋₃alkylene;

R³ is H, Br, Cl, F, I, C₁₋₆alkyl, SC₁₋₆alkyl or OC₁₋₆alkyl; and
X is independently NH or S;
wherein all alkyl and alkylene are optionally fluorosubstituted.

In an embodiment, R¹ is Cl or Br. In another embodiment, R¹ is C₁₋₆alkyl. In another embodiment R¹ is CF₃. In another embodiment, R¹ is SCH₃.

In another embodiment R² is C₁₋₆alkyl. In another embodiment R² is ethyl.

In an embodiment, R³ is C₁₋₆alkyl. In another embodiment R³ is CH₃ or CF₃. In another embodiment, R³ is OC₁₋₆alkyl. In another embodiment R³ is OCH₃ or OCF₃. In another embodiment, R³ is SC₁₋₆alkyl. In another embodiment, R³ is SCH₃. In yet another embodiment, R³ is F.

In an embodiment, the cannabinoid receptor is CB1. In another embodiment, the compound of Formula I is a negative allosteric modulator or allosteric inhibitor of cannabinoid receptor activity. In an embodiment, the compound of Formula I has an improved metabolic stability compared to certain prior art compounds.

In an embodiment, the cell is in vitro. In another embodiment, the cell is in vivo. In another embodiment, the cell may be derived from adipose tissue; lung tissue; gastrointestinal tissue including, for example, bowel and colon; breast tissue; ovarian tissue; prostate tissue; hepatic tissue; renal tissue; bladder tissue pancreas; brain tissue; or epithelial tissue.

As the compounds of Formula I as described above have been shown to be capable of modulating cannabinoid receptor activity, the compounds of the application are useful for treating diseases, disorders or conditions by modulating cannabinoid receptor activity. Accordingly, the present application also includes a method of treating a disease, disorder or condition by modulating cannabinoid receptor activity comprising administering a therapeutically effective amount of one or more compounds of Formula I as described above to a subject in need thereof.

The present application also includes a method of treating a mental disorder by modulating cannabinoid receptor activity comprising administering a therapeutically effective amount of one or more compounds of Formula I as described above to a subject in need thereof. In an embodiment, the mental disorder is anxiety, mania, bipolar disorder or schizophrenia. In an embodiment, the mental disorder is schizophrenia or bipolar disorder.

The present application also includes a method of treating a liver disorder by modulating cannabinoid receptor activity comprising administering a therapeutically effective amount of one or more compounds of Formula I as described above to a subject in need thereof. In an embodiment, the liver disorder is non-alcoholic fatty liver disease (NAFLD), non-alcoholic steatohepatitis (NASH), liver fibrosis of unknown origin, or non-alcoholic fatty liver disease (NAFLD) associated with metabolic syndrome. In an embodiment, the liver disorder is non-alcoholic fatty liver disease (NAFLD) or non-alcoholic steatohepatitis (NASH). In another embodiment, the liver disorder is induced by antipsychotic medication. In yet another embodiment, the liver disorder is in combination with a mental disorder.

The present application also includes a method of treating a disease, disorder or condition by modulating cannabinoid receptor activity comprising administering a therapeutically effective amount of one or more compounds of Formula I as described above in combination with another known agent useful for treatment of a disease, disorder or condition treatable by modulating cannabinoid receptor activity to a subject in need thereof.

In an embodiment, the subject is a mammal. In an embodiment, the subject is human.

The present application includes a compound of Formula Ia or a pharmaceutically acceptable salt and/or solvate thereof:

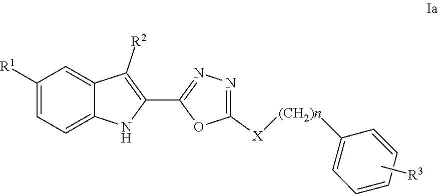

wherein:
R¹ is H, Br, Cl, F, I, C₁₋₆alkyl, SC₁₋₆alkyl or OC₁₋₆alkyl;
R² is H or C₁₋₆alkyl;
R³ is H, Br, Cl, F, I, C₁₋₆alkyl, SC₁₋₆alkyl or OC₁₋₆alkyl;
X is independently S or NH; and
n is 0, 1, 2 or 3;
wherein all alkyl are optionally fluorosubstituted, provided
when X is S and n is 0 or 1 then R¹, R² and R³ cannot all be H;
when X is S, n is 1, R¹ is Br and R² is H then R³ cannot be H, CH₃ or Cl.
when X is NH, n is 0, R² is H and R³ is H then R¹ cannot be H, Br or Cl;
when X is NH, n is 0, R² is CH₃ and R³ is H then R¹ cannot be H or Cl;
when X is NH, n is 0, R¹ is CH₃ and R² is CH₃ then R³ cannot be H;
when X is NH, n is 0, R¹ is OCH₃ and R³ is H then R² cannot be H or CH₃; and
when X is NH, n is 0, R¹ is OCH₂CH₃ and R³ is H then R² cannot be H.

In an embodiment, R¹ is Cl or Br. In another embodiment, R¹ is SCH₃ or CF₃.

In an embodiment R² is C₁₋₆alkyl. In another embodiment, R² is ethyl.

In an embodiment, R³ is C₁₋₆alkyl. In another embodiment R³ is CH₃ or CF₃. In another embodiment, R³ is OC₁₋₆alkyl. In another embodiment R³ is OCF₃ or OCH₃. In another embodiment, R³ is SCH₃. In yet another embodiment, R³ is F.

The present application also includes a pharmaceutical composition comprising one or more compounds of Formula I, or a pharmaceutically acceptable salt, and/or solvate thereof, and a pharmaceutically acceptable carrier and/or diluent.

In an embodiment, the pharmaceutical composition further comprises an additional therapeutic agent.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### BRIEF DESCRIPTION OF THE DRAWINGS

The application will be described in greater detail herein below with reference to the drawings in which:

FIG. 1 shows a graph illustrating the effects of exemplary compound, ABM300, in inhibiting the maximum level of stimulation (Eₘₐₓ) caused by the cannabinoid agonist CP55,940, as measured using the β-arrestin recruitment assay. The graph shows that ABM300 is a highly potent allosteric modulator of cannabinoid receptor activity and reduces the level of stimulation (Eₘₐₓ, efficacy) by 50% at a concentration of close to 1 nM (IC₅₀=1 nM, as calculated by GraphPad Prism). Each symbol represents the mean percentage of stimulation above basal ±S.E.M (n=2-3).

FIG. 2: shows a graph demonstrating that exemplary compound, ABM300 (10 mg/kg) reduces hyperactivity. Total distance traveled (cm) in open field (OF) test in (A) WT (F1: C57Bl/6J x 129S1/SvlmJ) and GluN1 KD mice and (B) WT (C57Bl/6J) and DATKO mice, balanced for sex, treated with either vehicle (1:1:18-Tween80:95% ethanol:saline) or ABM300 (10 mg/kg). Data shown as mean±SEM, *p<0.05, ***p<0.001, ****p<0.0001, two-way ANOVA, multiple comparisons, post-hoc Bonferroni's test.

FIG. 3 shows a graph demonstrating that exemplary compound, ABM300 (10 mg/kg) reduces abnormal stereotypic behaviours. Total stereotypic movements measured in open field (OF) test in (A) WT (F1: C57Bl/6J x 129S1/SvlmJ) and GluN1KD mice and (B) WT (C57Bl/6J) and DATKO mice, balanced for sex, treated with either vehicle (1:1:18-Tween80:95% ethanol:saline) or ABM300 (10 mg/kg). Data shown as mean±SEM, *p<0.05, **p<0.01, ****p<0.0001, two-way ANOVA, multiple comparisons, post-hoc Bonferroni's test.

FIG. 4 shows a graph demonstrating that exemplary compound, ABM300 (10 mg/kg) reduces abnormal vertical exploration (risk-taking behaviour). Total vertical activity measured in open field (OF) test in (A) WT (F1: C57Bl/6J x 129S1/SvlmJ) and GluN1KD mice and (B) WT (C57Bl/6J) and DATKO mice, balanced for sex, treated with either vehicle (1:1:18-Tween80:95% ethanol:saline) or ABM300 (10 mg/kg). Data shown as mean±SEM, ***p<0.001, ****p<0.0001, two-way ANOVA, multiple comparisons, post-hoc Bonferroni's test.

FIG. 5 shows a graph demonstrating that exemplary compound, ABM300 (10 mg/kg) rescues sensorimotor gating deficits in DATKO mice. Pre-pulse inhibition (PPI) measured in (A) WT (F1: C57Bl/6J x 129S1/SvlmJ) and GluN1 KD mice and (B) WT (C57Bl/6J) and DATKO mice, balanced for sex, treated with either vehicle (1:1:18-Tween80:95% ethanol:saline) or ABM300 (10 mg/kg). Data shown as mean±SEM, *p<0.05, **p<0.01, ***p<0.001, two-way ANOVA, multiple comparisons, post-hoc Bonferroni's test within each decibel pre-pulse

FIG. 6 shows hepatic triglycerides measured from livers of mice fed a HFD diet for 8 weeks and treated with either 4 weeks of daily vehicle (n=8) or 10 mg/kg exemplary compound ABM300 (n=9) injections. Hepatic triglycerides were measured using an ethanolic-KOH saponification method for lysing liver tissue and hydrolyzing triglyceride molecules to release free glycerol.

FIG. 7 shows serum alanine aminotransferase (ALT) levels from mice fed a HFD diet for 8 weeks and treated with 4 weeks of daily vehicle (n=8) or 10 mg/kg exemplary compound ABM300 (n=9) injections. Serum ALT, a marker for liver damage, was assessed using an Infinity™ ALT Liquid Stable Reagent (ThermoFisher).

FIG. 8 shows H&E stained liver sections from non-fasted mice fed an HFD diet for 8 weeks prior to daily treatment with either vehicle or exemplary compound ABM300 (10 mg/kg) for 4 weeks. A) A representative vehicle-treated liver is characterized by extensive cytoplasmic clearing indicative of both microvesicular and macrovesicular steatosis. B) A representative ABM300-treated liver displays cytoplasmic clearing characteristic of glycogen accumulation that is seen in non-fasted mice. Formalin-fixed livers were sectioned at 10 mM and stained using standard H&E staining.

FIG. 9 shows il Red O (ORO) stained liver sections from non-fasted mice fed an HFD diet for 8 weeks prior to daily treatment with vehicle or exemplary compound ABM300 (10 mg/kg) for 4 weeks. A) A representative vehicle-treated liver is characterized by regions of large ORO-stained vacuoles. B) A representative ABM300-treated liver displays smaller ORO-stained vacuoles. Frozen OCT-fixed livers were cryosectioned at 8 mM and stained using a conventional ORO staining technique, which stains for neutral lipids.

### DETAILED DESCRIPTION

### 1. Definitions

Unless otherwise indicated, the definitions and embodiments described in this and other sections are intended to be applicable to all embodiments and aspects of the application herein described for which they are suitable as would be understood by a person skilled in the art. Unless otherwise specified within this application or unless a person skilled in the art would understand otherwise, the nomenclature used in this application generally follows the examples and rules stated in “Nomenclature of Organic Chemistry” (Pergamon Press, 1979), Sections A, B, C, D, E, F, and H. Optionally, a name of a compound may be generated using a chemical naming program: ACD/ChemSketch, Version 5.09/September 2001, Advanced Chemistry Development, Inc., Toronto, Canada.

The term “compound of the application” or “compound of the present application” and the like as used herein refers to a compound of Formula I or Ia, and pharmaceutically acceptable salts and/or solvates thereof.

The term “composition of the application” or “composition of the present application” and the like as used herein refers to a composition comprising one or more compounds the application and at least one additional ingredient.

As used in the present application, the singular forms “a”, “an” and “the” include plural references unless the content clearly dictates otherwise. For example, an embodiment including “a compound” should be understood to present certain aspects with one compound, or two or more additional compounds.

In embodiments comprising an “additional” or “second” component, such as an additional or second compound, the second component as used herein is chemically different from the other components or first component. A “third” component is different from the other, first, and second components, and further enumerated or “additional” components are similarly different.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

The term “consisting” and its derivatives, as used herein, are intended to be closed terms that specify the presence of the stated features, elements, components, groups, integers, and/or steps, but exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

The term “suitable” as used herein means that the selection of the particular compound or conditions would depend on the specific synthetic manipulation to be performed, and the identity of the species to be transformed, but the selection would be well within the skill of a person trained in the art. All method steps described herein are to be conducted under conditions sufficient to provide the desired product. A person skilled in the art would understand that all reaction conditions, including, for example, reaction solvent, reaction time, reaction temperature, reaction pressure, reactant ratio and whether or not the reaction should be performed under an anhydrous or inert atmosphere, can be varied to optimize the yield of the desired product and it is within their skill to do so.

In embodiments of the present application, the compounds described herein may have at least one asymmetric center. Where compounds possess more than one asymmetric center, they may exist as diastereomers. It is to be understood that all such isomers and mixtures thereof in any proportion are encompassed within the scope of the present application. It is to be further understood that while the stereochemistry of the compounds may be as shown in any given compound listed herein, such compounds may also contain certain amounts (for example, less than 20%, suitably less than 10%, more suitably less than 5%) of compounds of the present application having alternate stereochemistry. It is intended that any optical isomers, as separated, pure or partially purified optical isomers or racemic mixtures thereof are

The compounds of the present application may also exist in different tautomeric forms and it is intended that any tautomeric forms which the compounds form are included within the scope of the present application.

The compounds of the present application may further exist in varying polymorphic forms and it is contemplated that any polymorphs which form are included within the scope of the present application.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

The term “alkyl” as used herein, whether it is used alone or as part of another group, means straight or branched chain, saturated alkyl groups. The number of carbon atoms that are possible in the referenced alkyl group are indicated by the prefix “Cₙ₁₋ₙ₂”. For example, the term C₁₋₆alkyl means an alkyl group having 1, 2, 3, 4, 5 or 6 carbon atoms. All alkyl groups are optionally fluorosubstituted unless otherwise stated.

The term “alkenyl” as used herein, whether it is used alone or as part of another group, means straight or branched chain, unsaturated alkyl groups containing at least one double bond. The number of carbon atoms that are possible in the referenced alkyl group are indicated by the prefix “Cₙ₁₋ₙ₂”. For example, the term C₂₋₆alkenyl means an alkenyl group having 2, 3, 4, 5 or 6 carbon atoms.

The term “alkylene” as used herein, whether it is used alone or as part of another group, means a straight or branched chain, saturated alkylene group, that is, a saturated carbon chain that contains substituents on two of its ends. The number of carbon atoms that are possible in the referenced alkylene group are indicated by the prefix “Cₙ₁₋ₙ₂”. For example, the term C₁₋₆alkylene means an alkylene group having 1, 2, 3, 4, 5 or 6 carbon atoms. All alkylene groups are optionally fluorosubstituted unless otherwise stated.

The term “halo” as used herein refers to a halogen atom and includes fluoro, chloro, bromo and iodo.

The term “optionally substituted” refers to groups, structures, or molecules that are either unsubstituted or are substituted with one or more substituents.

The term “fluorosubstituted” refers to the substitution of one or more, including all, hydrogens in a referenced group with fluorine.

The term “protecting group” or “PG” and the like as used herein refers to a chemical moiety which protects or masks a reactive portion of a molecule to prevent side reactions in those reactive portions of the molecule, while manipulating or reacting a different portion of the molecule. After the manipulation or reaction is complete, the protecting group is removed under conditions that do not degrade or decompose the remaining portions of the molecule. The selection of a suitable protecting group can be made by a person skilled in the art. Many conventional protecting groups are known in the art, for example as described in “Protective Groups in Organic Chemistry” McOmie, J. F. W. Ed., Plenum Press, 1973, in Greene, T. W. and Wuts, P. G. M., “Protective Groups in Organic Synthesis”, John Wiley & Sons, 3^{rd} Edition, 1999 and in Kocienski, P. Protecting Groups, 3rd Edition, 2003, Georg Thieme Verlag (The Americas).

The term “cell” as used herein refers to a single cell or a plurality of cells and includes a cell either in a cell culture or in a subject.

The term “subject” as used herein includes all members of the animal kingdom including mammals, and suitably refers to humans. Thus the methods and uses of the present application are applicable to both human therapy and veterinary applications.

The term “pharmaceutically acceptable” means compatible with the treatment of subjects, for example humans.

The term “pharmaceutically acceptable carrier” means a non-toxic solvent, dispersant, excipient, adjuvant or other material which is mixed with the active ingredient in order to permit the formation of a pharmaceutical composition, i.e., a dosage form capable of administration to a subject.

The term “pharmaceutically acceptable salt” means either an acid addition salt or a base addition salt which is suitable for, or compatible with the treatment of subjects.

An acid addition salt suitable for, or compatible with, the treatment of subjects is any non-toxic organic or inorganic acid addition salt of any basic compound. Basic compounds that form an acid addition salt include, for example, compounds comprising an amine group. Illustrative inorganic acids which form suitable salts include hydrochloric, hydrobromic, sulfuric, nitric and phosphoric acids, as well as acidic metal salts such as sodium monohydrogen orthophosphate and potassium hydrogen sulfate. Illustrative organic acids which form suitable salts include mono-, di- and tricarboxylic acids. Illustrative of such organic acids are, for example, acetic, trifluoroacetic, propionic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, mandelic, salicylic, 2-phenoxybenzoic, p-toluenesulfonic acid and other sulfonic acids such as methanesulfonic acid, ethanesulfonic acid and 2-hydroxyethanesulfonic acid. Either the mono- or di-acid salts can be formed, and such salts can exist in either a hydrated, solvated or substantially anhydrous form. In general, acid addition salts are more soluble in water and various hydrophilic organic solvents, and generally demonstrate higher melting points in comparison to their free base forms. The selection criteria for the appropriate salt will be known to one skilled in the art. Other non-pharmaceutically acceptable salts such as but not limited to oxalates may be used, for example in the isolation of compounds of the application for laboratory use, or for subsequent conversion to a pharmaceutically acceptable acid addition salt.

A base addition salt suitable for, or compatible with, the treatment of subjects is any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide as well as ammonia. Illustrative organic bases which form suitable salts include aliphatic, alicyclic or aromatic organic amines such as isopropylamine, methylamine, trimethylamine, picoline, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, EGFRaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. [See, for example, S. M. Berge, et al., “Pharmaceutical Salts,” J. Pharm. Sci. 1977, 66, 1-19]. The selection of the appropriate salt may be useful so that an ester functionality, if any, elsewhere in a compound is not hydrolyzed. The selection criteria for the appropriate salt will be known to one skilled in the art.

Prodrugs of the compounds of the present application may be, for example, conventional esters formed with available hydroxy, thiol, amino or carboxyl groups. Some common esters which have been utilized as prodrugs are phenyl esters, aliphatic (C₁-C₂₄) esters, acyloxymethyl esters, carbamates and amino acid esters.

The term “solvate” as used herein means a compound, or a salt or prodrug of a compound, wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate”.

The term “inert organic solvent” as used herein refers to a solvent that is generally considered as non-reactive with the functional groups that are present in the compounds to be combined together in any given reaction so that it does not interfere with or inhibit the desired synthetic transformation. Organic solvents are typically non-polar and dissolve compounds that are non soluble in aqueous solutions.

The term “treating” or “treatment” as used herein and as is well understood in the art, means an approach for obtaining beneficial or desired results, including clinical results. Beneficial or desired clinical results can include, but are not limited to alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. “Treating” and “treatment” as used herein also include prophylactic treatment. For example, a subject with early cancer can be treated to prevent progression, or alternatively a subject in remission can be treated with a compound or composition of the application to prevent recurrence. Treatment methods comprise administering to a subject a therapeutically effective amount of one or more of the compounds of the application and optionally consist of a single administration, or alternatively comprise a series of administrations. For example, in an embodiment, the compounds of the application may be administered at least once a week. In an embodiment, the compounds may be administered to the subject from about one time per three weeks, or about one time per week to about once daily for a given treatment. In another embodiment, the compounds are administered 2, 3, 4, 5 or 6 times daily. The length of the treatment period depends on a variety of factors, such as the severity of the disease, disorder or condition, the age of the subject, the concentration and/or the activity of the compounds of the application, and/or a combination thereof. It will also be appreciated that the effective dosage of the compound used for the treatment may increase or decrease over the course of a particular treatment regime. Changes in dosage may result and become apparent by standard diagnostic assays known in the art. In some instances, chronic administration may be required. For example, the compounds are administered to the subject in an amount and for duration sufficient to treat the patient.

“Palliating” a disease or disorder means that the extent and/or undesirable clinical manifestations of a disorder or a disease state are lessened and/or time course of the progression is slowed or lengthened, as compared to not treating the disorder.

The term “prevention” or “prophylaxis”, or synonym thereto, as used herein refers to a reduction in the risk or probability of a patient becoming afflicted with a disease, disorder or condition modulated by cannabinoid receptor activity or treatable by modulation of cannabinoid receptor activity or manifesting a symptom associated with a disease, disorder or condition modulated by cannabinoid receptor activity inhibition or treatable by modulation of cannabinoid receptor activity.

The “treating a disease, disorder or condition by modulating cannabinoid receptor activity” as used herein refers to a disease, disorder or condition treatable by modulating cannabinoid receptor activity and particularly using a negative allosteric modulator, or allosteric inhibitor, of cannabinoid receptor activity, such as a compound of the application herein described.

The term “modulating cannabinoid receptor activity” as used herein means that the disease, disorder or condition to be treated is affected by, and/or has some biological basis, either direct or indirect, that includes aberrant cannabinoid receptor activity, in particular, increased cannabinoid receptor activity or, also, decreased cannabinoid receptor activity such as results from mutation or splice variation and the like. These diseases respond favourably when cannabinoid receptor activity associated with the disease is modulated by one or more of the compounds of the application.

As used herein, the term “effective amount” or “therapeutically effective amount” means an amount of a compound, or one or more compounds, of the application that is effective, at dosages and for periods of time necessary to achieve the desired result. For example, in the context of treating a disease, disorder or condition by modulating cannabinoid receptor activity or treatable by modulating cannabinoid receptor activity, an effective amount is an amount that, for example, modulates cannabinoid receptor activity, compared to the activity without administration of the one or more compounds. Effective amounts may vary according to factors such as the disease state, age, sex and/or weight of the subject. The amount of a given compound that will correspond to such an amount will vary depending upon various factors, such as the given drug or compound, the pharmaceutical formulation, the route of administration, the type of condition, disease or disorder, the identity of the subject being treated, and the like, but can nevertheless be routinely determined by one skilled in the art. The effective amount is one that following treatment therewith manifests as an improvement in or reduction of any disease symptom. When the disease is cancer, amounts that are effective can cause a reduction in the number, growth rate, size and/or distribution of tumours.

The term “administered” as used herein means administration of a therapeutically effective amount of a compound, or one or more compounds, or a composition of the application to a cell either in cell culture or in a subject.

The term “mental disorder” as used herein means a disease, disorder or condition that is characterized by a behavioral or mental pattern that causes significant distress or impairment of personal functioning. A “mental disorder” can be also called a “mental illness” or a “psychiatric disorder”, all of which can be used interchangeably herein. Mental disorder includes but is not limited to anxiety disorder, mania, schizophrenia or bipolar disorder.

The term “liver disorder” as used herein means a disease, disorder or condition that causes liver inflammation or damage, and may affect liver function. A liver disorder includes is not limited to a non-alcoholic fatty liver disease (NAFLD), non-alcoholic steatohepatitis (NASH) or liver fibrosis of unknown origin.

### II. Methods and Uses of the Application

Indazole-oxadiazole compounds as described herein have been shown to be modulators of cannabinoid receptor activity.

Accordingly, the present application includes a method for modulating cannabinoid receptor activity in a cell comprising administering to the cell an effective amount of one or more compounds of Formula I or a pharmaceutically acceptable salt and/or solvate thereof:

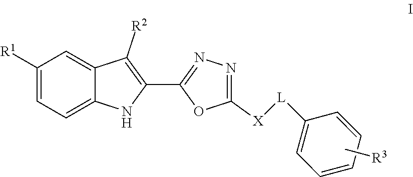

wherein:
R¹ is H, Br, Cl, F, I, C₁₋₆alkyl, SC₁₋₆alkyl or OC₁₋₆alkyl;
R² is H or C₁₋₆alkyl;

### L is C₀₋₃alkylene;

R³ is H, Br, Cl, F, I, C₁₋₆alkyl, SC₁₋₆alkyl or OC₁₋₆alkyl; and
X is independently NH or S;
wherein all alkyl and alkylene are optionally fluorosubstituted.

The application also includes a use of one or more compounds of Formula I as defined above for modulating cannabinoid receptor activity in a cell as well as a use of one or more compounds of Formula I as defined above in the preparation of a medicament for modulating cannabinoid receptor activity in a cell. The application further includes one or more compounds of Formula I as defined above for use in modulating cannabinoid receptor activity in a cell.

In an embodiment, R¹ is Cl or Br. In another embodiment, R¹ is C₁₋₆alkyl. In another embodiment R¹ is CF₃. In another embodiment, R¹ is SCH₃.

In another embodiment R² is C₁₋₆alkyl. In another embodiment R² is ethyl.

In an embodiment, R³ is C₁₋₆alkyl. In another embodiment R³ is CH₃ or CF₃. In another embodiment, R³ is OC₁₋₆alkyl. In another embodiment R³ is OCH₃ or OCF₃. In another embodiment, R³ is SC₁₋₆alkyl. In another embodiment, R³ is SCH₃. In yet another embodiment, R³ is F.

In an embodiment, the compound of Formula I is selected from the compounds listed below:

| Compound | | |
|---|---|---|
| I.D. | Example # | Structure |
| ABM300 | 2 | 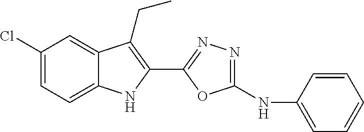 [SMILES: CCc1c(-c2nnc(Nc3ccccc3)o2)[nH]c2ccc(Cl)cc12] |
| ABM301 | 23 | 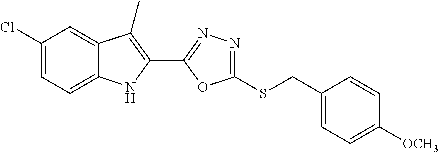 [SMILES: COc1ccc(CSc2nnc(-c3[nH]c4ccc(Cl)cc4c3C)o2)cc1] |
| ABM305 | 26 | 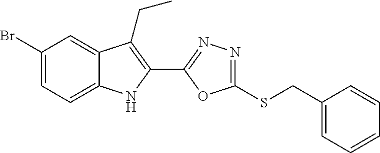 [SMILES: CCc1c(-c2nnc(SCc3ccccc3)o2)[nH]c2ccc(Br)cc12] |
| ABM309 | 27 | 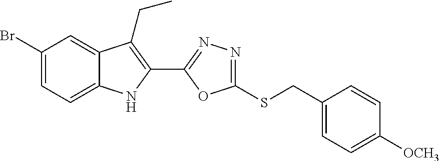 [SMILES: CCc1c(-c2nnc(SCc3ccc(OC)cc3)o2)[nH]c2ccc(Br)cc12] |
| ABM310 | 3 | 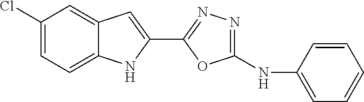 [SMILES: Clc1ccc2[nH]c(-c3nnc(Nc4ccccc4)o3)cc2c1] |
| ABM311 | 4 | 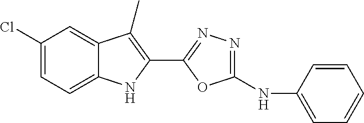 [SMILES: Cc1c(-c2nnc(Nc3ccccc3)o2)[nH]c2ccc(Cl)cc12] |
| ABM312 | 5 | 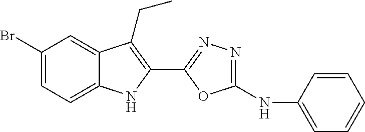 [SMILES: CCc1c(-c2nnc(Nc3ccccc3)o2)[nH]c2ccc(Br)cc12] |
| ABM313 | 7 | 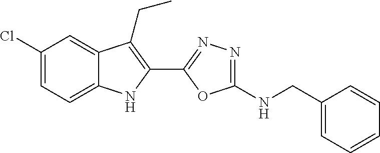 [SMILES: CCc1c(-c2nnc(NCc3ccccc3)o2)[nH]c2ccc(Cl)cc12] |
| ABM314 | 8 | 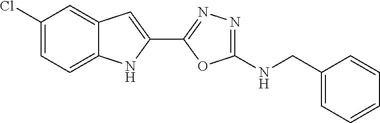 [SMILES: Clc1ccc2[nH]c(-c3nnc(NCc4ccccc4)o3)cc2c1] |
| ABM315 | 9 | 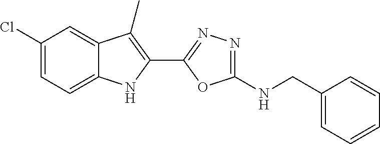 [SMILES: Cc1c(-c2nnc(NCc3ccccc3)o2)[nH]c2ccc(Cl)cc12] |
| ABM316 | 10 | 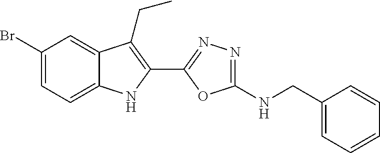 [SMILES: CCc1c(-c2nnc(NCc3ccccc3)o2)[nH]c2ccc(Br)cc12] |
| ABM317 | 12 | 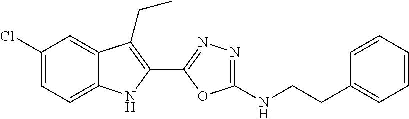 [SMILES: CCc1c(-c2nnc(NCCc3ccccc3)o2)[nH]c2ccc(Cl)cc12] |
| ABM318 | 13 | 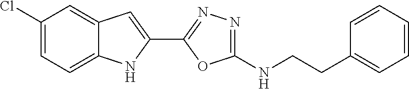 [SMILES: Clc1ccc2[nH]c(-c3nnc(NCCc4ccccc4)o3)cc2c1] |
| ABM319 | 14 | 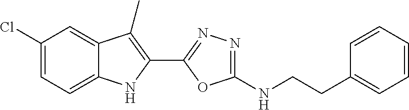 [SMILES: Cc1c(-c2nnc(NCCc3ccccc3)o2)[nH]c2ccc(Cl)cc12] |
| ABM320 | 15 | 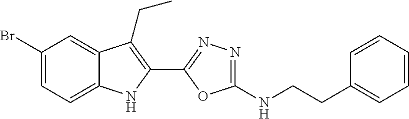 [SMILES: CCc1c(-c2nnc(NCCc3ccccc3)o2)[nH]c2ccc(Br)cc12] |
| ABM321 | 28 | 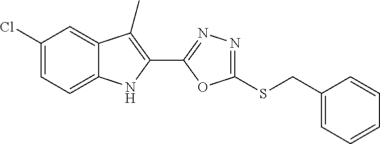 [SMILES: Cc1c(-c2nnc(SCc3ccccc3)o2)[nH]c2ccc(Cl)cc12] |
| ABM322 | 29 | 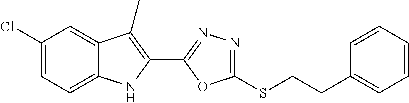 [SMILES: Cc1c(-c2nnc(SCCc3ccccc3)o2)[nH]c2ccc(Cl)cc12] |
| ABM323 | 30 | 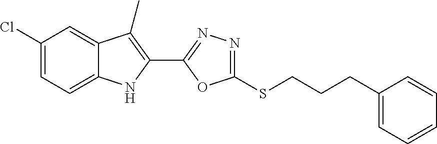 [SMILES: Cc1c(-c2nnc(SCCCc3ccccc3)o2)[nH]c2ccc(Cl)cc12] |
| ABM324 | 31 | 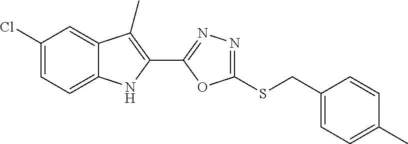 [SMILES: Cc1ccc(CSc2nnc(-c3[nH]c4ccc(Cl)cc4c3C)o2)cc1] |
| ABM325 | 32 | 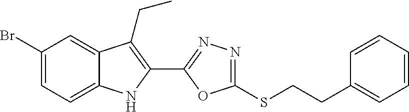 [SMILES: CCc1c(-c2nnc(SCCc3ccccc3)o2)[nH]c2ccc(Br)cc12] |
| ABM326 | 33 | 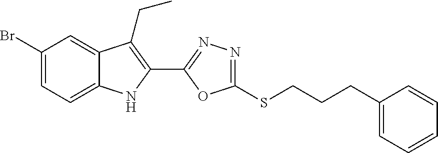 [SMILES: CCc1c(-c2nnc(SCCCc3ccccc3)o2)[nH]c2ccc(Br)cc12] |
| ABM327 | 34 | 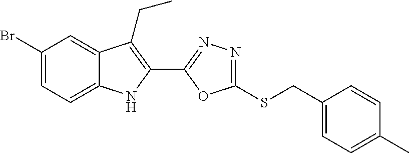 [SMILES: CCc1c(-c2nnc(SCc3ccc(C)cc3)o2)[nH]c2ccc(Br)cc12] |
| ABM328 | 37 | 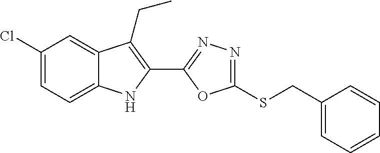 [SMILES: CCc1c(-c2nnc(SCc3ccccc3)o2)[nH]c2ccc(Cl)cc12] |
| ABM329 | 38 | 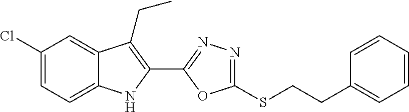 [SMILES: CCc1c(-c2nnc(SCCc3ccccc3)o2)[nH]c2ccc(Cl)cc12] |
| ABM330 | 39 | 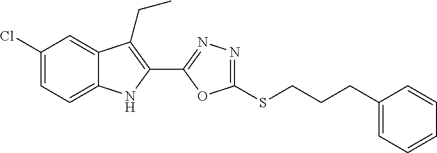 [SMILES: CCc1c(-c2nnc(SCCCc3ccccc3)o2)[nH]c2ccc(Cl)cc12] |
| ABM331 | 40 | 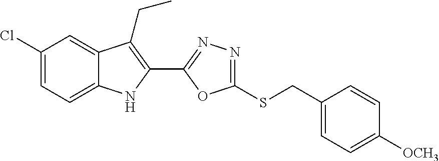 [SMILES: CCc1c(-c2nnc(SCc3ccc(OC)cc3)o2)[nH]c2ccc(Cl)cc12] |
| ABM332 | 41 | 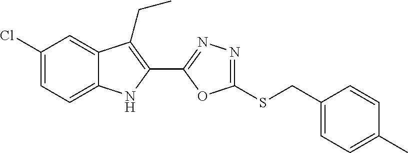 [SMILES: CCc1c(-c2nnc(SCc3ccc(C)cc3)o2)[nH]c2ccc(Cl)cc12] |
| ABM333 | 44 | 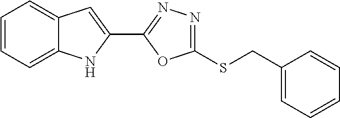 [SMILES: c1ccc(CSc2nnc(-c3cc4ccccc4[nH]3)o2)cc1] |
| ABM334 | 45 | 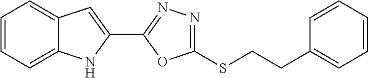 [SMILES: c1ccc(CCSc2nnc(-c3cc4ccccc4[nH]3)o2)cc1] |
| ABM335 | 17 | 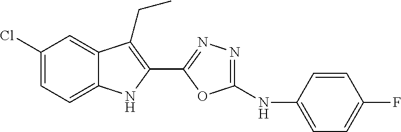 [SMILES: CCc1c(-c2nnc(Nc3ccc(F)cc3)o2)[nH]c2ccc(Cl)cc12] |
| ABM336 | 18 | 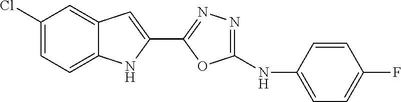 [SMILES: Fc1ccc(Nc2nnc(-c3cc4cc(Cl)ccc4[nH]3)o2)cc1] |
| ABM337 | 19 | 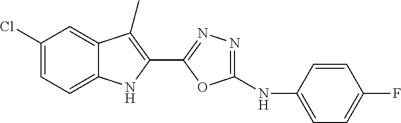 [SMILES: Cc1c(-c2nnc(Nc3ccc(F)cc3)o2)[nH]c2ccc(Cl)cc12] |
| ABM338 | 20 | 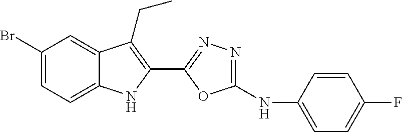 [SMILES: CCc1c(-c2nnc(Nc3ccc(F)cc3)o2)[nH]c2ccc(Br)cc12] |
| ABM339 | 46 | 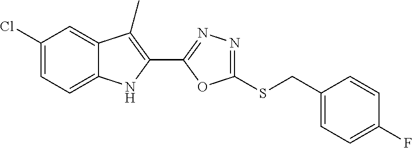 [SMILES: Cc1c(-c2nnc(SCc3ccc(F)cc3)o2)[nH]c2ccc(Cl)cc12] |
| ABM340 | 47 | 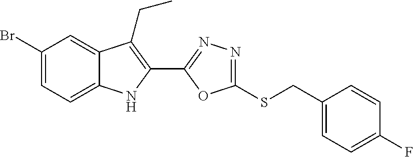 [SMILES: CCc1c(-c2nnc(SCc3ccc(F)cc3)o2)[nH]c2ccc(Br)cc12] |

or a pharmaceutically acceptable salt and/or solvate thereof.

In an embodiment, the cannabinoid receptor is CB1. In another embodiment, the compound of Formula I is an allosteric modulator of cannabinoid receptor activity. In yet another embodiment, the compound of Formula I is a negative allosteric modulator of cannabinoid receptor activity.

In an embodiment, the cell is in vitro. In another embodiment, the cell is in vivo. In another embodiment, the cell may be derived from adipose tissue; lung tissue; gastrointestinal tissue including, for example, bowel and colon; breast tissue; ovarian tissue; prostate tissue; hepatic tissue; renal tissue; bladder tissue pancreas; brain tissue; or epithelial tissue.

As the compounds of Formula I as described above have been shown to be capable of modulating cannabinoid receptor activity, the compounds of the application are useful for treating diseases, disorders or conditions by modulating cannabinoid receptor activity. Therefore, the compounds of the present application are useful as medicaments. Accordingly, the present application includes compounds of Formula I as described above for use as a medicament.

Accordingly, the present application also includes a method of treating a disease, disorder or condition by modulating cannabinoid receptor activity comprising administering a therapeutically effective amount of one or more compounds of Formula I as described above to a subject in need thereof.

The present application also includes a use of one or more compounds of Formula I as described above for treatment of a disease, disorder or condition by modulating cannabinoid receptor activity as well as a use of one or more compounds of the application for the preparation of a medicament for treatment of a disease, disorder or condition by modulating cannabinoid receptor activity. The application further includes one or more compounds of the Formula I as described above for use in treating a disease, disorder or condition by modulating cannabinoid receptor activity.

In an embodiment the disease, disorder or condition that is treated by modulating cannabinoid receptor activity is a psychiatric disease, disorder or condition such as anxiety, mania, bipolar disorder or schizophrenia; a liver disease, disorder or condition such as non-alcoholic fatty liver disease (NAFLD) or non-alcoholic steatohepatitis (NASH), liver fibrosis of unknown origin, or non-alcoholic fatty liver disease (NAFLD) associated with metabolic syndrome; metabolic syndrome; type-2 diabetes; dyslipidaemia; obesity; eating disorder; cardiovascular disease or disease, disorder or condition associated with cardiovascular disease such as hypertension, congestive heart failure, cardiac hypertrophy, peripheral artery disease, atherosclerosis, stroke, kidney disease, myocardial infarction, steatohepatitis, and cardiotoxicity associated with chemotherapy; a disease, disorder or condition characterized by an addiction component such as addiction or withdrawal, such as smoking addiction and/or smoking withdrawal, alcohol addiction and/or alcohol withdrawal, drug addiction and/or drug withdrawal; a bone disease, disorder or condition such as osteoporosis, Paget's disease of bone, or bone related cancer; breast cancer; a disease, disorder or condition characterized by an inflammatory or an autoimmune component such as rheumatoid arthritis, inflammatory bowel disease, or psoriasis; and/or a disease, disorder or condition characterized by impairment of memory and/or loss of cognitive function such as memory impairment, loss of cognitive function, Parkinson's disease, Alzheimer's disease, or dementia.

Compounds of the application have been demonstrated to be effective in animal models of mental disorder such as schizophrenia or bipolar disorder. Therefore, in an embodiment, the disease, disorder or condition is a mental disorder. Accordingly, the present application also includes a method of treating a mental disorder by modulating cannabinoid receptor activity comprising administering a therapeutically effective amount of one or more compounds of Formula I as described above to a subject in need thereof. The present application also includes a use of one or more compounds of Formula I as described above for treatment of a mental disorder by modulating cannabinoid receptor activity as well as a use of one or more compounds of the application for the preparation of a medicament for treatment of a mental disorder by modulating cannabinoid receptor activity. The application further includes one or more compounds of the Formula I as described above for use in treating a mental disorder by modulating cannabinoid receptor activity. In an embodiment, the mental disorder is anxiety, mania, bipolar disorder or schizophrenia. In an embodiment, the mental disorder is schizophrenia or bipolar disorder. In another embodiment, the treatment is in an amount effective to ameliorate at least one symptom of the mental disorder, for example, reduced hyperactivity or risk-taking behavior, in a subject in need of such treatment.

Compounds of the application have been demonstrated to be effective in animal models of liver disease. Therefore, in an embodiment, the disease, disorder or condition is a liver disorder. Accordingly, the present application also includes a method of treating a liver disorder by modulating cannabinoid receptor activity comprising administering a therapeutically effective amount of one or more compounds of Formula I as described above to a subject in need thereof. The present application also includes a use of one or more compounds of Formula I as described above for treatment of a liver disorder by modulating cannabinoid receptor activity as well as a use of one or more compounds of the application for the preparation of a medicament for treatment of a liver disorder by modulating cannabinoid receptor activity. The application further includes one or more compounds of the Formula I as described above for use in treating a liver disorder by modulating cannabinoid receptor activity. In another embodiment, the treatment is in an amount effective to ameliorate at least one symptom of the liver disorder, for example, reduced triglyceride levels or serum alanine aminotransferase (ALT), in a subject in need of such treatment. In an embodiment the liver disorder is non-alcoholic fatty liver disease (NAFLD), non-alcoholic steatohepatitis (NASH), liver fibrosis of unknown origin, or non-alcoholic fatty liver disease (NAFLD) associated with metabolic syndrome. In an embodiment, the liver disorder is non-alcoholic fatty liver disease (NAFLD) or non-alcoholic steatohepatitis (NASH). In another embodiment, the liver disorder is induced by antipsychotic medication. In yet another embodiment, the liver disorder is in combination with a mental disorder.

The present application also includes a method of treating a disease, disorder or condition by modulating cannabinoid receptor activity comprising administering a therapeutically effective amount of one or more compounds of Formula I as described above in combination with another known agent useful for treatment of a disease, disorder or condition treatable by modulating cannabinoid receptor activity to a subject in need thereof.

The present application also includes a use of one or more compounds of Formula I as described above in combination with another known agent useful for treatment of a disease, disorder or condition treatable by modulating cannabinoid receptor activity for treatment of a disease, disorder or condition by modulating cannabinoid receptor activity. The present application also includes a use of one or more compounds of the application in combination with another known agent useful for treatment of a disease, disorder or condition treatable by modulating cannabinoid receptor activity for the preparation of a medicament for treatment of a disease, disorder or condition by modulating cannabinoid receptor activity. The application further includes one or more compounds of the Formula I as described above in combination with another known agent useful for treatment of a disease, disorder or condition treatable by modulating cannabinoid receptor activity for use in treating a disease, disorder or condition by modulating cannabinoid receptor activity. In an embodiment, the other known agent useful for treatment of a disease, disorder or condition treatable by modulating cannabinoid receptor activity is used in the treatment of type-2 diabetes and obesity, such as insulin and insulin analogues; dipeptidyl peptidase-4 (DPP-4) inhibitors; glucagon-like peptide-1 analogues; hypoglycemic agents, such as alpha-glucosidase inhibitors; biguanides; sulfonyl ureas; thiazolidinediones; weight loss therapies, such as appetite suppressing agents, serotonin reuptake inhibitors, noradrenaline reuptake inhibitors, β₃-adrenoceptor agonists, and lipase inhibitors; used in the treatment of cardiovascular disease and disfunction, such as diuretics; angiotensin-converting enzyme (ACE) inhibitors; angiotensin II antagonists; beta-blockers; calcium antagonists, such as nifedipine; HMG-CoA-reductase inhibitors, such as statins; other lipid modulating agents, such as fibrates; bile acid-binding resins; drugs used to treat cardiac dysfunction, such as digoxin, aldosterone antagonists, and organic nitrates; used to assist smoking cessation, such as norepinephrine-dopamine reuptake inhibitors, such as bupropion; used in the treatment of bone diseases and disorders, such as anti-resportive agents, such as bisphosphonates; anabolic agents, such as parathyroid hormone; RANKL inhibitors, such as denosumab; estrogen replacement and selective estrogen receptor modulators, such as raloxifene; used in the treatment of breast cancer, such as compounds which modulate tubulyin polymerization, such as paclitaxel; targeted therapies, such as antibodies against specific cell surface markers on tumour cells, such as antibodies against the HER2 oncoprotein, such as trastuzumab; used in the treatment of a disease or disorder with an inflammatory or autoimmune component, such as non-steroidal anti-inflammatory drugs (NSAIDs); disease-modifying anti-rheumatic drugs (DMARDs), such as immunosuppressants; anti-TNF agents, such as infliximab, etanercept, and adalimumab; and anti B-cell therapies, such as rituximab; used in the treatment of a psychiatric disease or disorder, such as GABAA modulators, such as benzodiazepines; 5HT_{1A} receptor agonists, such as buspirone; beta blockers; antipsychotics, such as dopamine receptor blockers and other drugs which modulate monoamine receptors, transporters or metabolism, for example, tricyclic antidepressants, selective serotonin reuptake inhibitors, and monoamine oxidase inhibitors; lithium; and anti-epileptic drugs, such as those which block sodium channels, those which block T-type calcium channels, or those which block GABA transaminase or reuptake, including phenytoin, carbamazepine, valproate and vigabatrin; or used in the treatment of a disease or disorder characterized by impairment of memory and/or loss of cognitive function, such as dopamine agonists and anticholinesterases.

In an embodiment, a compound of the present application is administered with another agent simultaneously or sequentially in separate unit dosage forms or together in a single unit dosage form.

In an embodiment, the subject is a mammal. In an embodiment, the subject is human. In another embodiment, the cannabinoid receptor is CB1.

### III. Compounds and Compositions of the Application

Compounds of the present application were prepared and were found to modulate uncontrolled and/or abnormal cellular activities affected directly or indirectly by cannabinoid receptor activity. In particular, compounds of the present application exhibited activity as negative modulators of cannabinoid receptor activity, and are therefore useful in therapy, for example for the treatment of mental disorders or liver disorders.

In an embodiment, the application includes certain novel compounds of Formula I. Accordingly, the present application includes a compound of Formula Ia or a pharmaceutically acceptable salt and/or solvate thereof:

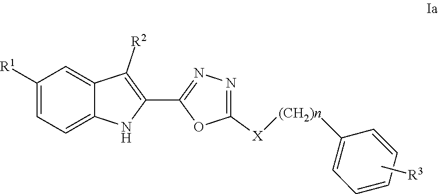

wherein:
R¹ is H, Br, Cl, F, or I, C₁₋₆alkyl, SC₁₋₆alkyl or OC₁₋₆alkyl;
R² is H or C₁₋₆alkyl;
R³ is H. H, Br, Cl, F, I, C₁₋₆alkyl, SC₁₋₆alkyl or OC₁₋₆alkyl;
X is independently S or NH; and
n is 0, 1, 2 or 3;
wherein all alkyl are optionally fluorosubstituted,
provided
when X is S and n is 0 or 1 then R¹, R² and R³ cannot all be H;
when X is S, n is 1, R¹ is Br and R² is H then R³ cannot be H, CH₃ or Cl;
when X is NH, n is 0, R² is H and R³ is H then R¹ cannot be H, Br or Cl;
when X is NH, n is 0, R² is CH₃ and R³ is H then R¹ cannot be H or Cl;
when X is NH, n is 0, R¹ is CH₃ and R² is CH₃ then R³ cannot be H;
when X is NH, n is 0, R¹ is OCH₃ and R³ is H then R² cannot be H or CH₃; and
when X is NH, n is 0, R¹ is OCH₂CH₃ and R³ is H then R² cannot be H.

In an embodiment, R¹ is Cl or Br. In another embodiment, R¹ is SCH₃ or CF₃.

In another embodiment R² is C₁₋₆alkyl. In another embodiment R² is ethyl.

In an embodiment, R³ is C₁₋₆alkyl. In an embodiment, R³ is CH₃ or CF₃. In another embodiment, R³ is OC₁₋₆alkyl. In an embodiment, R³ is OCF₃ or OCH₃. In another embodiment, R³ is SCH₃. In yet another embodiment, R³ is F. In an embodiment, the compound of Formula Ia has an improved metabolic stability compared to certain prior art compounds.

In an embodiment, the compound of Formula Ia is selected from the compounds listed below, or a pharmaceutically acceptable salt, and/or solvate thereof:

| Compound | | |
|---|---|---|
| I.D. | Example # | Structure |
| ABM300 | 2 | 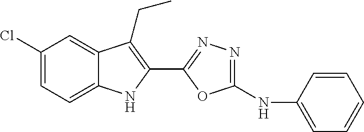 [SMILES: CCc1c(-c2nnc(Nc3ccccc3)o2)[nH]c2ccc(Cl)cc12] |
| ABM301 | 23 | 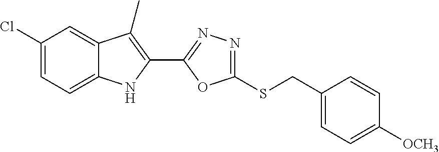 [SMILES: COc1ccc(CSc2nnc(-c3[nH]c4ccc(Cl)cc4c3C)o2)cc1] |
| ABM305 | 26 | 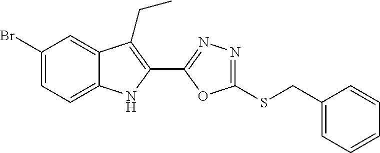 [SMILES: CCc1c(-c2nnc(SCc3ccccc3)o2)[nH]c2ccc(Br)cc12] |
| ABM309 | 27 | 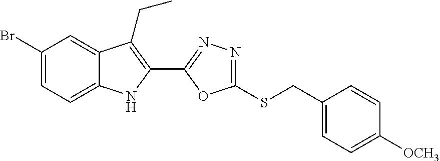 [SMILES: CCc1c(-c2nnc(SCc3ccc(OC)cc3)o2)[nH]c2ccc(Br)cc12] |
| ABM312 | 5 | 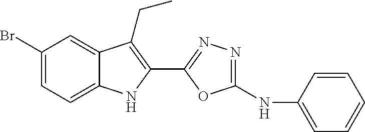 [SMILES: CCc1c(-c2nnc(Nc3ccccc3)o2)[nH]c2ccc(Br)cc12] |
| ABM313 | 7 | 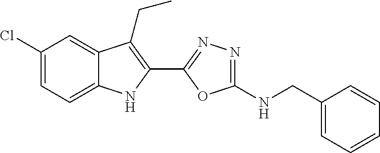 [SMILES: CCc1c(-c2nnc(NCc3ccccc3)o2)[nH]c2ccc(Cl)cc12] |
| ABM314 | 8 | 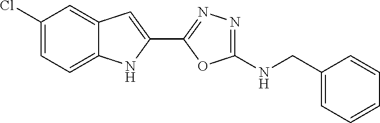 [SMILES: Clc1ccc2[nH]c(-c3nnc(NCc4ccccc4)o3)cc2c1] |
| ABM315 | 9 | 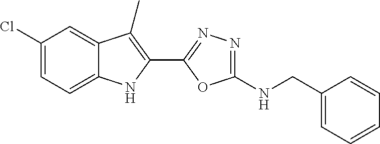 [SMILES: Cc1c(-c2nnc(NCc3ccccc3)o2)[nH]c2ccc(Cl)cc12] |
| ABM316 | 10 | 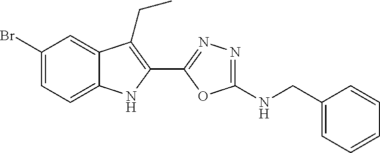 [SMILES: CCc1c(-c2nnc(NCc3ccccc3)o2)[nH]c2ccc(Br)cc12] |
| ABM317 | 12 | 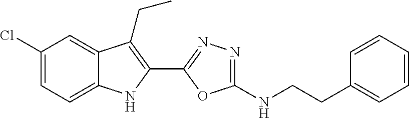 [SMILES: CCc1c(-c2nnc(NCCc3ccccc3)o2)[nH]c2ccc(Cl)cc12] |
| ABM318 | 13 | 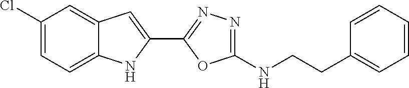 [SMILES: Clc1ccc2[nH]c(-c3nnc(NCCc4ccccc4)o3)cc2c1] |
| ABM319 | 14 | 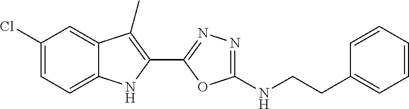 [SMILES: Cc1c(-c2nnc(NCCc3ccccc3)o2)[nH]c2ccc(Cl)cc12] |
| ABM320 | 15 | 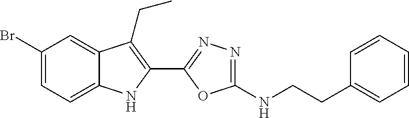 [SMILES: CCc1c(-c2nnc(NCCc3ccccc3)o2)[nH]c2ccc(Br)cc12] |
| ABM321 | 28 | 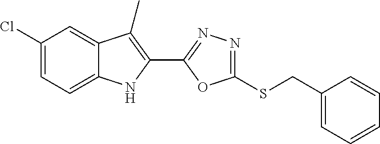 [SMILES: Cc1c(-c2nnc(SCc3ccccc3)o2)[nH]c2ccc(Cl)cc12] |
| ABM322 | 29 | 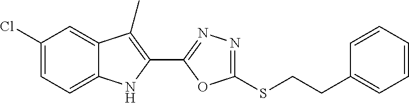 [SMILES: Cc1c(-c2nnc(SCCc3ccccc3)o2)[nH]c2ccc(Cl)cc12] |
| ABM323 | 30 | 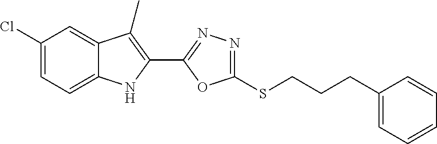 [SMILES: Cc1c(-c2nnc(SCCCc3ccccc3)o2)[nH]c2ccc(Cl)cc12] |
| ABM324 | 31 | 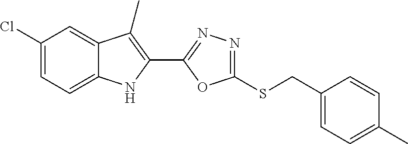 [SMILES: Cc1ccc(CSc2nnc(-c3[nH]c4ccc(Cl)cc4c3C)o2)cc1] |
| ABM325 | 32 | 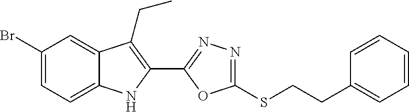 [SMILES: CCc1c(-c2nnc(SCCc3ccccc3)o2)[nH]c2ccc(Br)cc12] |
| ABM326 | 33 | 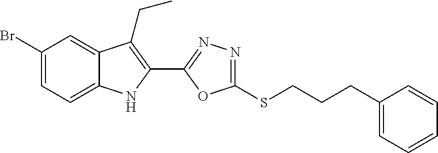 [SMILES: CCc1c(-c2nnc(SCCCc3ccccc3)o2)[nH]c2ccc(Br)cc12] |
| ABM327 | 34 | 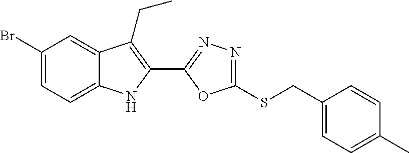 [SMILES: CCc1c(-c2nnc(SCc3ccc(C)cc3)o2)[nH]c2ccc(Br)cc12] |
| ABM328 | 37 | 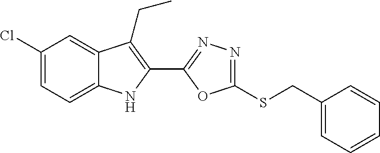 [SMILES: CCc1c(-c2nnc(SCc3ccccc3)o2)[nH]c2ccc(Cl)cc12] |
| ABM329 | 38 | 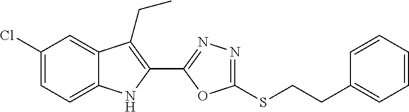 [SMILES: CCc1c(-c2nnc(SCCc3ccccc3)o2)[nH]c2ccc(Cl)cc12] |
| ABM330 | 39 | 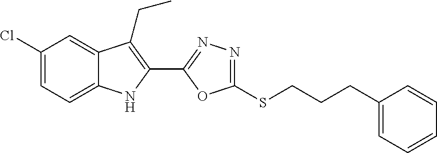 [SMILES: CCc1c(-c2nnc(SCCCc3ccccc3)o2)[nH]c2ccc(Cl)cc12] |
| ABM331 | 40 | 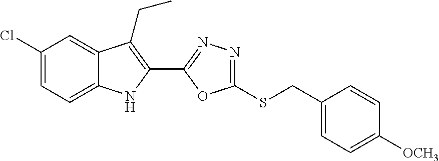 [SMILES: CCc1c(-c2nnc(SCc3ccc(OC)cc3)o2)[nH]c2ccc(Cl)cc12] |
| ABM332 | 41 | 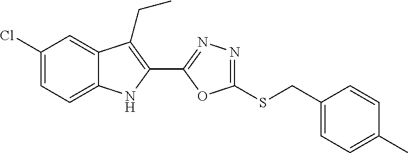 [SMILES: CCc1c(-c2nnc(SCc3ccc(C)cc3)o2)[nH]c2ccc(Cl)cc12] |
| ABM334 | 45 | 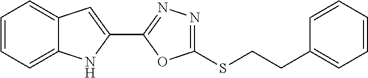 [SMILES: c1ccc(CCSc2nnc(-c3cc4ccccc4[nH]3)o2)cc1] |
| ABM335 | 17 | 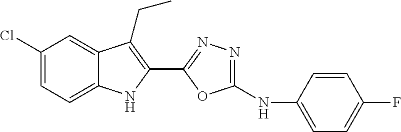 [SMILES: CCc1c(-c2nnc(Nc3ccc(F)cc3)o2)[nH]c2ccc(Cl)cc12] |
| ABM336 | 18 | 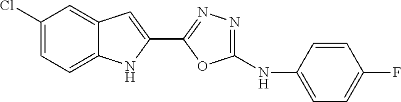 [SMILES: Fc1ccc(Nc2nnc(-c3cc4cc(Cl)ccc4[nH]3)o2)cc1] |
| ABM337 | 19 | 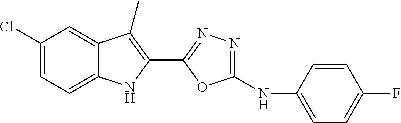 [SMILES: Cc1c(-c2nnc(Nc3ccc(F)cc3)o2)[nH]c2ccc(Cl)cc12] |
| ABM338 | 20 | 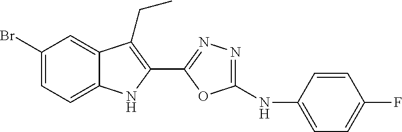 [SMILES: CCc1c(-c2nnc(Nc3ccc(F)cc3)o2)[nH]c2ccc(Br)cc12] |
| ABM339 | 46 | 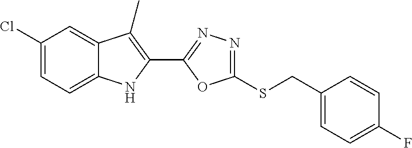 [SMILES: Cc1c(-c2nnc(SCc3ccc(F)cc3)o2)[nH]c2ccc(Cl)cc12] |
| ABM340 | 47 | 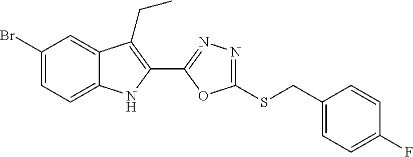 [SMILES: CCc1c(-c2nnc(SCc3ccc(F)cc3)o2)[nH]c2ccc(Br)cc12] |

The compounds of the application may also be provided in combination. Conversely, various features of the application, which are, for brevity, described in the context of a single embodiment, may also be provided separately or in any suitable sub-combination. All combinations of the embodiments pertaining to the chemical groups represented by the variables (e.g., —R¹, —R², —R³, etc.) are specifically embraced by the present application and are disclosed herein just as if each and every combination was individually and explicitly disclosed, to the extent that such combinations embrace compounds that are stable compounds (i.e., compounds that can be isolated, characterized, and tested for biological activity). In addition, all sub-combinations of the chemical groups listed in the embodiments describing such variables are also specifically embraced by the present application and are disclosed herein just as if each and every such sub-combination of chemical groups was individually and explicitly disclosed herein.

In an embodiment, the compounds of the application, as described herein, may also be provided in substantially purified form and/or in a form substantially free from contaminants. In an embodiment, the compound is in substantially purified form and/or in a form substantially free from contaminants. In another embodiment, the compound is in a substantially purified form with a purity of least 50% by weight, e.g., at least 60% by weight, e.g., at least 70% by weight, e.g., at least 80% by weight, e.g., at least 90% by weight, e.g., at least 95% by weight, e.g., at least 97% by weight, e.g., at least 98% by weight, e.g., at least 99% by weight.

Unless specified, the substantially purified form refers to a compound of the application in any stereoisomeric or enantiomeric form. For example, in an embodiment, the substantially purified form refers to a mixture of stereoisomers, i.e., purified with respect to other compounds. In an embodiment, the substantially purified form refers to one stereoisomer, e.g., optically pure stereoisomer. In an embodiment, the substantially purified form refers to a mixture of enantiomers. In an embodiment, the substantially purified form refers to an equimolar mixture of enantiomers (i.e., a racemic mixture, a racemate). In an embodiment, the substantially purified form refers to one enantiomer, e.g., optically pure enantiomer.

In another embodiment, the compound of the application is in a form substantially free from contaminants wherein the contaminants represent no more than 50% by weight, e.g., no more than 40% by weight, e.g., no more than 30% by weight, e.g., no more than 20% by weight, e.g., no more than 10% by weight, e.g., no more than 5% by weight, e.g., no more than 3% by weight, e.g., no more than 2% by weight, e.g., no more than 1% by weight.

Unless specified, the contaminants refer to other compounds, that is, other than stereoisomers or enantiomers. In an embodiment, the contaminants refer to other compounds and other stereoisomers. In an embodiment, the contaminants refer to other compounds and the other enantiomer.

In another embodiment, the compound of the application is in a substantially purified form with an optical purity of at least 60% (i.e., 60% of the compound, on a molar basis, is the desired stereoisomer or enantiomer, and 40% is undesired stereoisomer(s) or enantiomer), e.g., at least 70%, e.g., at least 80%, e.g., at least 90%, e.g., at least 95%, e.g., at least 97%, e.g., at least 98%, e.g., at least 99%.

The compounds of the present application are suitably formulated in a conventional manner into compositions using one or more carriers. Accordingly, the present application also includes a composition comprising one or more compounds of the application and a carrier. The compounds of the application are suitably formulated into pharmaceutical compositions for administration to subjects in a biologically compatible form suitable for administration in vivo. Accordingly, the present application further includes a pharmaceutical composition comprising one or more compounds of the application and a pharmaceutically acceptable carrier. In particular, the pharmaceutical application comprises one or more compounds of Formula Ia and a pharmaceutically acceptable carrier.

The compounds of the application may be administered to a subject in a variety of forms depending on the selected route of administration, as will be understood by those skilled in the art. A compound of the application may be administered, for example, by oral, parenteral, buccal, sublingual, nasal, rectal, patch, pump or transdermal administration and the pharmaceutical compositions formulated accordingly. Administration can be by means of a pump for periodic or continuous delivery.

Parenteral administration includes intravenous, intra-arterial, intraperitoneal, subcutaneous, intramuscular, transepithelial, nasal, intrapulmonary (for example, by use of an aerosol), intrathecal, rectal and topical (including the use of a patch or other transdermal delivery device) modes of administration. Parenteral administration may be by continuous infusion over a selected period of time. Conventional procedures and ingredients for the selection and preparation of suitable compositions are described, for example, in Remington's Pharmaceutical Sciences (2000-20th edition) and in The United States Pharmacopeia: The National Formulary (USP 24 NF19) published in 1999.

A compound of the application may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the compound may be incorporated with excipient and used in the form of ingestible tablets, buccal tablets, troches, capsules, caplets, pellets, granules, lozenges, chewing gum, powders, syrups, elixirs, wafers, aqueous solutions and suspensions, and the like. In the case of tablets, carriers that are used include lactose, corn starch, sodium citrate and salts of phosphoric acid. Pharmaceutically acceptable excipients include binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. In the case of tablets, capsules, caplets, pellets or granules for oral administration, pH sensitive enteric coatings, such as Eudragits™ designed to control the release of active ingredients are optionally used. Oral dosage forms also include modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. Timed-release compositions can be formulated, e.g. liposomes or those wherein the active compound is protected with differentially degradable coatings, such as by microencapsulation, multiple coatings, etc. Liposome delivery systems include, for example, small unilamellar vesicles, large unilamellar vesicles and multilamellar vesicles. Liposomes can be formed from a variety of phospholipids, such as cholesterol, stearylamine or phosphatidylcholines. For oral administration in a capsule form, useful carriers or diluents include lactose and dried corn starch.

Liquid preparations for oral administration may take the form of, for example, solutions, syrups or suspensions, or they are suitably presented as a dry product for constitution with water or other suitable vehicle before use. When aqueous suspensions and/or emulsions are administered orally, the compound of the application is suitably suspended or dissolved in an oily phase that is combined with emulsifying and/or suspending agents. If desired, certain sweetening and/or flavoring and/or coloring agents may be added. Such liquid preparations for oral administration may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid). Useful diluents include lactose and high molecular weight polyethylene glycols.

It is also possible to freeze-dry the compounds of the application and use the lyophilizates obtained, for example, for the preparation of products for injection.

A compound of the application may also be administered parenterally. Solutions of a compound of the application can be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. For parenteral administration, sterile solutions of the compounds of the application are usually prepared, and the pH of the solutions are suitably adjusted and buffered. For intravenous use, the total concentration of solutes should be controlled to render the preparation isotonic. For ocular administration, ointments or droppable liquids may be delivered by ocular delivery systems known to the art such as applicators or eye droppers. Such compositions can include mucomimetics such as hyaluronic acid, chondroitin sulfate, hydroxypropyl methylcellulose or polyvinyl alcohol, preservatives such as sorbic acid, EDTA or benzyl chromium chloride, and the usual quantities of diluents or carriers. For pulmonary administration, diluents or carriers will be selected to be appropriate to allow the formation of an aerosol.

The compounds of the application may be formulated for parenteral administration by injection, including using conventional catheterization techniques or infusion. Formulations for injection may be presented in unit dosage form, e.g., in ampoules or in multi-dose containers, with an added preservative. The compositions may take such forms as sterile suspensions, solutions or emulsions in oily or aqueous vehicles, and may contain formulating agents such as suspending, stabilizing and/or dispersing agents. In all cases, the form must be sterile and must be fluid to the extent that easy syringability exists. Alternatively, the compounds of the application are suitably in a sterile powder form for reconstitution with a suitable vehicle, e.g., sterile pyrogen-free water, before use.

Compositions for nasal administration may conveniently be formulated as aerosols, drops, gels and powders.

For intranasal administration or administration by inhalation, the compounds of the application are conveniently delivered in the form of a solution, dry powder formulation or suspension from a pump spray container that is squeezed or pumped by the patient or as an aerosol spray presentation from a pressurized container or a nebulizer. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device such as a single dose nasal inhaler or an aerosol dispenser fitted with a metering valve which is intended for disposal after use. Where the dosage form comprises an aerosol dispenser, it will contain a propellant which can be a compressed gas such as compressed air or an organic propellant such as fluorochlorohydrocarbon. Suitable propellants include but are not limited to dichlorodifluoromethane, trichlorofluoromethane, dichlorotetrafluoroethane, heptafluoroalkanes, carbon dioxide or another suitable gas. In the case of a pressurized aerosol, the dosage unit is suitably determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the application and a suitable powder base such as lactose or starch. The aerosol dosage forms can also take the form of a pump-atomizer.

Compositions suitable for buccal or sublingual administration include tablets, lozenges, and pastilles, wherein the active ingredient is formulated with a carrier such as sugar, acacia, tragacanth, or gelatin and glycerine. Compositions for rectal administration are conveniently in the form of suppositories containing a conventional suppository base such as cocoa butter.

Suppository forms of the compounds of the application are useful for vaginal, urethral and rectal administrations. Such suppositories will generally be constructed of a mixture of substances that is solid at room temperature but melts at body temperature. The substances commonly used to create such vehicles include but are not limited to theobroma oil (also known as cocoa butter), glycerinated gelatin, other glycerides, hydrogenated vegetable oils, mixtures of polyethylene glycols of various molecular weights and fatty acid esters of polyethylene glycol. See, for example: Remington's Pharmaceutical Sciences, 16th Ed., Mack Publishing, Easton, Pa., 1980, pp. 1530-1533 for further discussion of suppository dosage forms.

Compounds of the application may also be coupled with soluble polymers as targetable drug carriers. Such polymers can include polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, compounds of the application may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

The compounds of the application including pharmaceutically acceptable salts, solvates and prodrugs thereof are suitably used on their own but will generally be administered in the form of a pharmaceutical composition in which the one or more compounds of the application (the active ingredient) is in association with a pharmaceutically acceptable carrier. Depending on the mode of administration, the pharmaceutical composition will comprise from about 0.05 wt % to about 99 wt % or about 0.10 wt % to about 70 wt %, of the active ingredient (one or more compounds of the application), and from about 1 wt % to about 99.95 wt % or about 30 wt % to about 99.90 wt % of a pharmaceutically acceptable carrier, all percentages by weight being based on the total composition.

Compounds of the application may be used alone or in combination with other known agents useful for treating diseases, disorders or conditions modulated by cannabinoid receptor activity inhibition, or that are treatable by modulation of cannabinoid receptor activity. When used in combination with other agents useful in treating diseases, disorders or conditions modulated by cannabinoid receptor activity inhibition, or that are treatable by modulation of cannabinoid receptor activity, it is an embodiment that the compounds of the application are administered contemporaneously with those agents. As used herein, “contemporaneous administration” of two substances to a subject means providing each of the two substances so that they are both biologically active in the individual at the same time. The exact details of the administration will depend on the pharmacokinetics of the two substances in the presence of each other, and can include administering the two substances within a few hours of each other, or even administering one substance within 24 hours of administration of the other, if the pharmacokinetics are suitable. Design of suitable dosing regimens is routine for one skilled in the art. In particular embodiments, two substances will be administered substantially simultaneously, i.e., within minutes of each other, or in a single composition that contains both substances. It is a further embodiment of the present application that a combination of agents is administered to a subject in a non-contemporaneous fashion. In an embodiment, a compound of the present application is administered with another therapeutic agent simultaneously or sequentially in separate unit dosage forms or together in a single unit dosage form. Accordingly, the present application provides a single unit dosage form comprising one or more compounds of the application (e.g. a compound of Formula I), an additional therapeutic agent, and a pharmaceutically acceptable carrier.

The dosage of compounds of the application can vary depending on many factors such as the pharmacodynamic properties of the compound, the mode of administration, the age, health and weight of the recipient, the nature and extent of the symptoms, the frequency of the treatment and the type of concurrent treatment, if any, and the clearance rate of the compound in the subject to be treated. One of skill in the art can determine the appropriate dosage based on the above factors. Compounds of the application may be administered initially in a suitable dosage that may be adjusted as required, depending on the clinical response. Dosages will generally be selected to maintain a serum level of compounds of the application from about 0.01 μg/cc to about 1000 μg/cc, or about 0.1 μg/cc to about 100 μg/cc. As a representative example, oral dosages of one or more compounds of the application will range between about 1 mg per day to about 1000 mg per day for an adult, suitably about 1 mg per day to about 500 mg per day, more suitably about 1 mg per day to about 200 mg per day. For parenteral administration, a representative amount is from about 0.001 mg/kg to about 10 mg/kg, about 0.01 mg/kg to about 10 mg/kg, about 0.01 mg/kg to about 1 mg/kg or about 0.1 mg/kg to about 1 mg/kg will be administered. For oral administration, a representative amount is from about 0.001 mg/kg to about 10 mg/kg, about 0.1 mg/kg to about 10 mg/kg, about 0.01 mg/kg to about 1 mg/kg or about 0.1 mg/kg to about 1 mg/kg. For administration in suppository form, a representative amount is from about 0.1 mg/kg to about 10 mg/kg or about 0.1 mg/kg to about 1 mg/kg. In an embodiment of the application, compositions are formulated for oral administration and the compounds are suitably in the form of tablets containing 0.25, 0.5, 0.75, 1.0, 5.0, 10.0, 20.0, 25.0, 30.0, 40.0, 50.0, 60.0, 70.0, 75.0, 80.0, 90.0, 100.0, 150, 200, 250, 300, 350, 400, 450, 500, 550, 600, 650, 700, 750, 800, 850, 900, 950 or 1000 mg of active ingredient per tablet. Compounds of the application may be administered in a single daily, weekly or monthly dose or the total daily dose may be divided into two, three or four daily doses.

To be clear, in the above, the term “a compound” also includes embodiments wherein one or more compounds are referenced.

### IV. Preparation of Compounds of the Application

Compounds of the present application can be prepared by various synthetic processes. The choice of particular structural features and/or substituents may influence the selection of one process over another. The selection of a particular process to prepare a given compound of Formula I is within the purview of the person of skill in the art. Some starting materials for preparing compounds of the present application are available from commercial chemical sources. Other starting materials, for example as described below, are readily prepared from available precursors using straightforward transformations that are well known in the art. In the Schemes below showing the preparation of compounds of the application, all variables are as defined in Formula I, unless otherwise stated,

Methods for the chemical synthesis of 5-(1H-indol-2-yl)-1,3,4-oxadiazol-2-amine and 5-(1H-indol-2-yl)-1,3,4-oxadiazole-2-thiol compounds (collectively referred to herein as indole-oxadiazoles) are described herein. These and/or other well-known methods may be modified and/or adapted in known ways in order to facilitate the synthesis of additional indole-oxadiazoles compounds (as described herein).

In an embodiment, an appropriate isothiocyanate, e.g. a phenylisothiocyanate, is reacted with an appropriate hydrazine, e.g. hydrazine monohydrate to give the desired thiosemicarbazide.

An example of such a method is shown in Scheme 1.

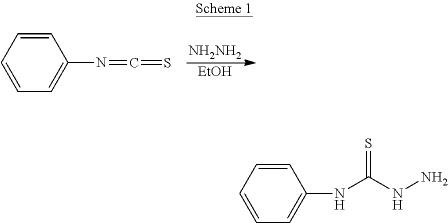

In an embodiment, the thiosemicarbazide is reacted with an appropriate indole-2-carboxylic acid, e.g. 5-chloro-3-ethyl-indole-2-carboxylate, in the presence of an appropriate coupling agent, e.g. EDC, to give the desired indole-1,3,4-oxadiazol-2-amine.

An example of such a method is shown in Scheme 2.

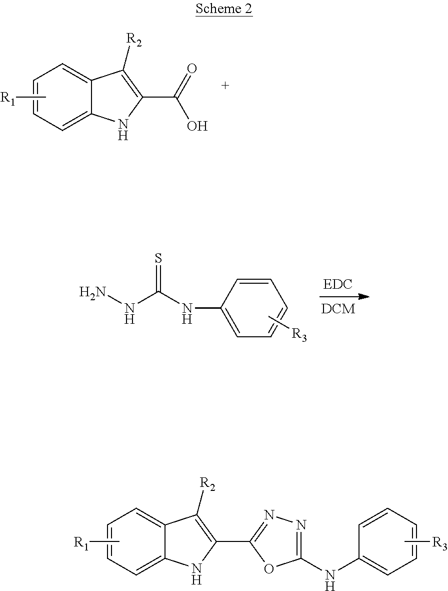

In an embodiment an indole-2-carbohydrazide can be prepared by the reaction of an appropriate indole-2-carboxylate ester, e.g. ethyl-5-chloro-3-ethylindole-2-carboxylate, with an appropriate hydrazine.

An example of such a method is shown in Scheme 3.

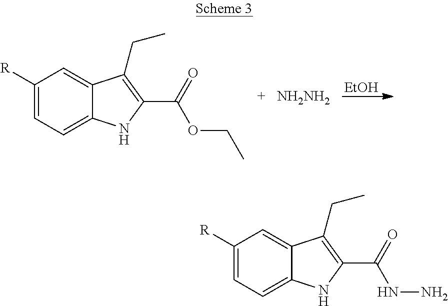

In an embodiment the indole-2-carbohydrazide can be reacted with carbon disulfide in the presence of a suitable base, e.g. aqueous potassium hydroxide, in a suitable solvent, e.g. ethanol. Acidification of the reaction mixture gives the desired (indol-2-yl)-1,3,4-oxadiazole-2-thiol.

An example of such a method is shown in Scheme 4.

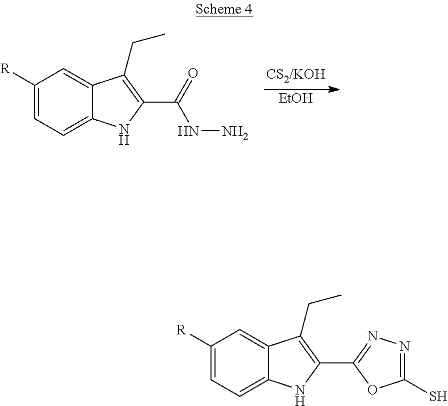

In an embodiment, the (indol-2-yl)-1,3,4-oxadiazole-2-thiol is reacted with a suitable aryl group, e.g. an arylalkylhalide, in the presence of a suitable base, e.g. potassium carbonate, to give the desired arylthiooxadiazole.

An example of such a method is shown in Scheme 5.

Scheme 5

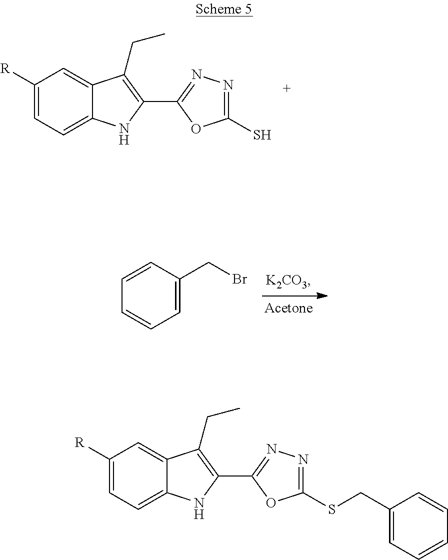

Salts of the compounds of the application are generally formed by dissolving the neutral compound in an inert organic solvent and adding either the desired acid or base and isolating the resulting salt by either filtration or other known means. “

It may be convenient or desirable to prepare, purify, and/or handle a corresponding salt of the compounds of the application, for example, a pharmaceutically-acceptable salt. Examples of pharmaceutically acceptable salts are discussed in Berge et al., 1977, “Pharmaceutically Acceptable Salts,” J. Pharm. Sci., Vol. 66, pp. 1-19.

Unless otherwise specified, a reference to a particular compound also includes salt forms thereof.

It may be convenient or desirable to prepare, purify, and/or handle a corresponding solvate of the compound. The term “solvate” is used herein in the conventional sense to refer to a complex of solute (e.g., compound, salt of compound) and solvent. If the solvent is water, the solvate may be conveniently referred to as a hydrate, for example, a hemi-hydrate, a mono-hydrate, a di-hydrate, a tri-hydrate, etc.

Unless otherwise specified, a reference to a particular compound also includes solvate (e.g., hydrate) forms thereof.

The formation of solvates of the compounds of the application will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art

Typical procedures for making and identifying suitable hydrates and solvates are well known to those in the art; see for example, pages 202-209 of K. J. Guillory, “Generation of Polymorphs, Hydrates, Solvates, and Amorphous Solids,” in: Polymorphism in Pharmaceutical Solids, ed. Harry G. Britain, Vol. 95, Marcel Dekker, Inc., New York, 1999.

Hydrates and solvates can be isolated and characterized by methods known in the art, such as, thermogravimetric analysis (TGA), TGA-mass spectroscopy, TGA-Infrared spectroscopy, powder X-ray diffraction (XRPD), Karl Fisher titration, high resolution X-ray diffraction, and the like. There are several commercial entities that provide quick and efficient services for identifying solvates and hydrates on a routine basis. Example companies offering these services include Wilmington PharmaTech (Wilmington, Del.), Avantium Technologies (Amsterdam) and Aptuit (Greenwich, Conn.).

For the avoidance of doubt, it is understood that the phrase “pharmaceutically acceptable salts and solvates thereof” and the phrase “pharmaceutically acceptable salt or solvate thereof” embrace pharmaceutically acceptable solvates (e.g., hydrates) of the compounds, pharmaceutically acceptable salts of the compounds, as well as pharmaceutically acceptable solvates (e.g., hydrates) of pharmaceutically acceptable salts of the compounds.

It may be convenient or desirable to prepare, purify, and/or handle the compound in the form of a prodrug. The term “prodrug,” as used herein, pertains to a compound which, when metabolized (e.g., in vivo), yields the desired active compound. Typically, the prodrug is inactive, or less active than the desired active compound, but may provide advantageous handling, administration, or metabolic properties.

For example, active compounds which have a hydroxyl or carboxylic acid group may be converted to prodrugs which are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

It may be convenient or desirable to prepare, purify, and/or handle the compound in a chemically protected form. The term “chemically protected form” is used herein in the conventional chemical sense and pertains to a compound in which one or more reactive functional groups are protected from undesirable chemical reactions under specified conditions (e.g., pH, temperature, radiation, solvent, and the like). In practice, well known chemical methods are employed to reversibly render unreactive a functional group, which otherwise would be reactive, under specified conditions. In a chemically protected form, one or more reactive functional groups are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, Protective Groups in Organic Synthesis (T. Greene and P. Wuts; 4th Edition; John Wiley and Sons, 2006).

A wide variety of such “protecting,” “blocking,” or “masking” methods are widely used and well known in organic synthesis. For example, a compound which has two nonequivalent reactive functional groups, both of which would be reactive under specified conditions, may be derivatized to render one of the functional groups “protected,” and therefore unreactive, under the specified conditions; so protected, the compound may be used as a reactant which has effectively only one reactive functional group. After the desired reaction (involving the other functional group) is complete, the protected group may be “deprotected” to return it to its original functionality.

For example, a hydroxy group may be protected as an ether (—OR) or an ester (—OC(═O)R), for example, as: a t-butyl ether; a benzyl, benzhydryl (diphenylmethyl), or trityl (triphenylmethyl) ether; a trimethylsilyl or t-butyldimethylsilyl ether; or an acetyl ester (—OC(═O)CH₃, —OAc).

For example, an aldehyde or ketone group may be protected as an acetal (R—CH(OR)₂) or ketal (R₂C(OR)₂), respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid.

For example, an amine group may be protected, for example, as an amide (—NRCO—R) or a urethane (—NRCO—OR), for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH—Fmoc), as a 6-nitroveratryloxy amide (—NH—Nvoc), as a 2-trimethylsilylethyloxy amide (—NH-Teoc), as a 2,2,2-trichloroethyloxy amide (—NH-Troc), as an allyloxy amide (—NH-Alloc), as a 2(-phenylsulfonyl)ethyloxy amide (—NH—Psec); or, in suitable cases (e.g., cyclic amines), as a nitroxide radical (>N—O.).

For example, a carboxylic acid group may be protected as an ester for example, as: an C₁₋₇alkyl ester (e.g., a methyl ester; a t-butyl ester); a C₁₋₇haloalkyl ester (e.g., a C₁₋₇trihaloalkyl ester); a triC₁₋₇alkylsilyl-C₁₋₇alkyl ester; or a C₅₋₂₀aryl-Cl-7alkyl ester (e.g., a benzyl ester; a nitrobenzyl ester); or as an amide, for example, as a methyl amide.

For example, a thiol group may be protected as a thioether (—SR), for example, as: a benzyl thioether; an acetamidomethyl ether (—S—CH₂NHC(═O)CH₃).

The following non-limiting examples are illustrative of the present application:

### EXAMPLES

### General Methods

### General Synthesis Method A: Synthesis of Thiosemicarbazides

To a solution of the appropriate isothiocyanate (14.36 mmol) in ethanol (25 mL), 60% hydrazine monohydrate (0.96 ml, 17.23 mmol) was added and the mixture was stirred at rt for 30 min. The precipitate formed was filtered off, washed with ethanol to provide the corresponding thiosemicarbazide.

### General Synthesis Method B: Synthesis of N-phenyl-1,3,4-oxadiazol-2-amines

A mixture of the indole-2-carboxylic acid (0.894 mmol, 1 eq), thiosemicarbazide (1.073 mmol, 1.2 eq) and EDC (0.51 g, 2.682 mmol, 3 eq) in DCM (25 mL) was stirred at rt for overnight. The reaction mixture was purified by silica gel column chromatography (EtOAc). After removal of the solvent, the residue was suspended in few mL of Et₂O, the precipitate filtered and washed with Et₂O to give the desired compound.

### General Synthesis Method C: Synthesis of indole-2-carbohydrazides

To a solution of indole ester (10 mmol) in absolute ethanol (40 mL), 80% hydrazine monohydrate (50 mmol) was added and the reaction mixture was refluxed overnight. After completion of the reaction, the solvent was concentrated by vacuo and the solid which precipitated upon cooling was collected by filtration. The collected precipitate was washed with cold ethanol, then Et₂O, and dried to provide indole hydrazides

### General Synthesis Method D: Synthesis of 5-(indol-2-yl)-1,3,4-oxadiazole-2-thiols

Indole-2-carbohydrazide (4 mmol) was dissolved or suspended in ethanol (20 mL). Carbon disulfide (0.48 g, 8 mmol) was then added to the reaction mixture followed by the addition of a solution of KOH (0.44 g, 8 mmol) in water (5 mL). The reaction mixture was heated at reflux with stirring for 5 h and the progress of the reaction was monitored by TLC. Ethanol was distilled off under reduced pressure and the residue was dissolved in H₂O and then acidified with 5% aqueous hydrochloric acid solution. The precipitate was filtered, washed with water, extracted with EtOAc. The organic layer was washed with water and brine, dried over MgSO₄, and concentrated in vacuo to afford a crude product which was purified by either column chromatography on silica using EtOAc/hexanes (2:3) or the residue was suspended in a few mL Et₂O then the formed precipitate was filtered and washed with Et₂O to give pure desired compound

### General Synthesis Method E: Synthesis of 2-(1H-indol-2-yl)-5-((benzyl)thio)-1,3,4-oxadiazoles

To a stirred solution of 5-(indol-2-yl)-1,3,4-oxadiazole-2-thiol (1 mmol, 0.268 g) and K₂CO₃ (0.12 g, 1.2 mmol) in acetone (25 mL) was added the appropriate halogen substituted compound (1.2 mmol). The resulting mixture was heated under reflux for 0.5 h and the reaction was monitored by TLC. After removal of the organic solvent in vacuo and addition of water, the residue was extracted by EtOAc and the organic layer was washed with saturated brine. Then the organic phase was dried over anhydrous MgSO₄ and the solvent was removed in vacuo. The purification of the crude product was done by either column chromatography on silica using EtOAc/hexanes (1:2) or the residue was suspended in a few mL Et₂O then the formed precipitate was filtered and washed with Et₂O to give pure desired compound

### Example 1: N-Phenylhydrazinecarbothioamide

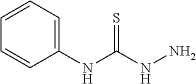 [SMILES: NNC(=S)Nc1ccccc1]

N-Phenylhydrazinecarbothioamide was prepared as a white solid from isothiocyanatobenzene and hydrazine monohydrate using General Method A. ¹H NMR (400 MHz, DMSO-d₆) δ 9.66 (s, 1H, NHCSNHNH₂), 9.13 (s, 1H, NHCSNHNH₂), 7.65 (d, J=8.0 Hz, 2H, Ar—H), 7.30 (t, J=7.8 Hz, 2H, Ar—H), 7.09 (t, J=7.4 Hz, 1H, Ar—H), 4.80 (s, 2H, NHCSNHNH₂). ¹³C NMR (101 MHz, DMSO-d₆) δ 179.38, 139.24, 128.08, 124.14, 123.54.

### Example 2: 5-(5-Chloro-3-ethyl-1H-indol-2-yl)-N-phenyl-1,3,4-oxadiazol-2-amine (ABM300)

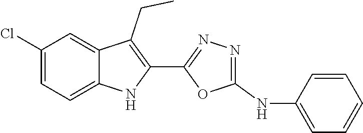 [SMILES: CCc1c(-c2nnc(Nc3ccccc3)o2)[nH]c2ccc(Cl)cc12]

5-(5-Chloro-3-ethyl-1H-indol-2-yl)-N-phenyl-1,3,4-oxadiazol-2-amine was prepared as a white solid from 5-chloro-3-ethyl-1H-indole-2-carboxylic acid and N-phenylhydrazinecarbothioamide using General Method B. ¹H NMR (400 MHz, DMSO-d₆) δ 11.88 (s, 1H, indole NH), 10.64 (s, 1H, NH), 7.70 (s, 1H, Ar—H), 7.63 (d, J=7.8 Hz, 2H, Ar—H), 7.45-7.32 (m, 3H, Ar—H), 7.20 (d, J=8.7 Hz, 1H, Ar—H), 7.02 (t, J=7.3 Hz, 1H, Ar—H), 3.04 (q, J=7.5 Hz, 2H, CH₂H₃), 1.23 (t, J=7.4 Hz, 3H, CH₂CH₃). ¹³C NMR (101 MHz, DMSO-d₆) δ 160.01, 153.77, 139.08, 135.85, 129.56, 128.66, 124.50, 124.23, 122.42, 119.95, 119.56, 119.06, 117.63, 114.01, 17.59, 16.06. HRESI-MS m/z calcd for [M+H]⁺ C₁₈H₁₆ClN₄O: 339.1007, found: 339.1010. mp 252-254° C.

### Example 3: 5-(5-Chloro-1H-indol-2-yl)-N-phenyl-1,3,4-oxadiazol-2-amine (ABM310)

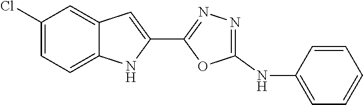 [SMILES: Clc1ccc2[nH]c(-c3nnc(Nc4ccccc4)o3)cc2c1]

5-(5-Chloro-1H-indol-2-yl)-N-phenyl-1,3,4-oxadiazol-2-amine was prepared as a white solid from 5-chloro-1H-indole-2-carboxylic acid and N-phenylhydrazinecarbothioamide using General Method B. ¹H NMR (400 MHz, DMSO-d₆) δ 12.33 (s, 1H, indole NH), 10.73 (s, 1H, NH), 7.75-6.93 (m, 9H, Ar—H). ¹³C NMR (101 MHz, DMSO-d₆) δ 160.06, 153.36, 138.96, 136.43, 129.56, 128.87, 125.07, 124.17, 123.65, 122.45, 120.69, 117.60, 114.05, 103.12. HRESI-MS m/z calcd for [M+H]⁺ C₁₆H₁₂ClN₄O: 311.0694, found: 311.0697. mp 272-274° C.

### Example 4: 5-(5-Chloro-3-methyl-1H-indol-2-yl)-N-phenyl-1,3,4-oxadiazol-2-amine (ABM311)

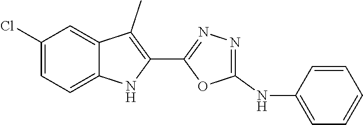 [SMILES: Cc1c(-c2nnc(Nc3ccccc3)o2)[nH]c2ccc(Cl)cc12]

5-(5-Chloro-3-methyl-1H-indol-2-yl)-N-phenyl-1,3,4-oxadiazol-2-amine was prepared as a white solid from 5-chloro-3-methyl-1H-indole-2-carboxylic acid and N-phenylhydrazinecarbothioamide using General Method B. ¹H NMR (400 MHz, DMSO-d₆) δ 11.89 (s, 1H, indole NH), 10.64 (s, 1H, NH), 7.70-7.60 (m, 3H, Ar—H), 7.44-7.29 (m, 3H, Ar—H), 7.20 (d, J=8.6 Hz, 1H, Ar—H), 7.02 (t, J=7.4 Hz, 1H, Ar—H), 2.54 (s, 3H, CH₃). ¹³C NMR (101 MHz, DMSO-d₆) δ 159.93, 153.92, 139.07, 135.76, 129.62, 129.56, 124.48, 124.27, 122.40, 120.28, 119.17, 117.60, 113.89, 113.18, 9.74. HRESI-MS m/z calcd for [M+H]⁺ C₁₇H₁₄ClN₄O: 325.0851, found: 325.0854. mp 268-270° C.

### Example 5: 5-(5-Bromo-3-ethyl-1H-indol-2-yl)-N-phenyl-1,3,4-oxadiazol-2-amine (ABM312)

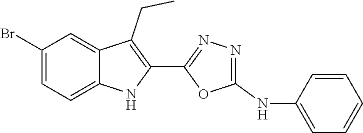 [SMILES: CCc1c(-c2nnc(Nc3ccccc3)o2)[nH]c2ccc(Br)cc12]

5-(5-Bromo-3-ethyl-1H-indol-2-yl)-N-phenyl-1,3,4-oxadiazol-2-amine was prepared as a white solid from 5-bromo-3-ethyl-1H-indole-2-carboxylic acid and N-phenylhydrazinecarbothioamide using General Method B. ¹H NMR (400 MHz, DMSO-d₆) δ 11.90 (s, 1H, indole NH), 10.64 (s, 1H, NH), 7.84 (s, 1H, Ar—H), 7.72-6.96 (m, 7H, Ar—H), 3.04 (q, J=7.4 Hz, 2H, CH₂CH₃), 1.23 (t, J=7.4 Hz, 3H, CH₂CH₃). ¹³C NMR (101 MHz, DMSO-d₆) δ 160.01, 153.75, 139.07, 136.08, 129.56, 129.36, 126.73, 122.44, 122.10, 119.85, 119.39, 117.64, 114.45, 112.41, 17.58, 16.08. HRESI-MS m/z calcd for [M+H]⁺ C₁₈H₁₆BrN₄O: 383.0502, found: 383.0504. mp 240-242° C.

### Example 6: N-Benzylhydrazinecarbothioamide

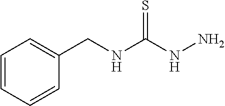 [SMILES: NNC(=S)NCc1ccccc1]

N-Benzylhydrazinecarbothioamide was prepared as a white solid from (isothiocyanatomethyl)benzene and hydrazine monohydrate using General Method A. ¹H NMR (400 MHz, DMSO-d₆) δ 8.75 (s, 1H, CH₂NH), 8.29 (s, 1H, NHNH₂), 7.36-7.27 (m, 4H, Ar—H), 7.25-7.20 (m, 1H, Ar—H), 4.74 (d, J=6.1 Hz, 2H, CH₂), 4.52 (s, 2H, NHNH₂). ¹³C NMR (101 MHz, DMSO-d₆) δ 181.58, 139.79, 128.11, 127.34, 126.68, 46.18.

### Example 7: N-Benzyl-5-(5-chloro-3-ethyl-1H-indol-2-yl)-1,3,4-oxadiazol-2-amine (ABM313)

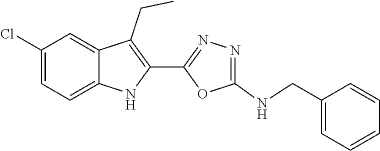 [SMILES: CCc1c(-c2nnc(NCc3ccccc3)o2)[nH]c2ccc(Cl)cc12]

N-Benzyl-5-(5-chloro-3-ethyl-1H-indol-2-yl)-1,3,4-oxadiazol-2-amine was prepared as a white solid from 5-chloro-3-ethyl-1H-indole-2-carboxylic acid and N-benzylhydrazinecarbothioamide using General Method B. ¹H NMR (400 MHz, DMSO-d₆) δ 11.77 (s, 1H, indole NH), 8.40 (t, J=6.2 Hz, 1H, NHCH₂), 7.66 (d, J=2.1 Hz, 1H, Ar—H), 7.44-7.32 (m, 5H, Ar—H), 7.27 (t, J=7.1 Hz, 1H, Ar—H), 7.18 (dd, J=8.7, 2.0 Hz, 1H, Ar—H), 4.48 (d, J=6.1 Hz, 2H, NHCH₂), 2.96 (q, J=7.5 Hz, 2H, CH₂CH₃), 1.17 (t, J=7.4 Hz, 3H, CH₂CH₃). ¹³C NMR (101 MHz, DMSO-d₆) δ 163.23, 153.24, 138.77, 135.21, 128.41, 128.28, 127.36, 127.19, 123.99, 123.49, 119.52, 118.73, 118.47, 113.46, 46.22, 17.10, 15.47. HRESI-MS m/z calcd for [M+H]⁺ C₁₉H₁₈ClN₄O: 353.1164, found: 353.1151. mp 216-218° C.

### Example 8: N-Benzyl-5-(5-chloro-1H-indol-2-yl)-1,3,4-oxadiazol-2-amine (ABM314)

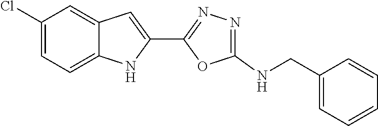 [SMILES: Clc1ccc2[nH]c(-c3nnc(NCc4ccccc4)o3)cc2c1]

N-Benzyl-5-(5-chloro-1H-indol-2-yl)-1,3,4-oxadiazol-2-amine was prepared as a white solid from 5-chloro-1H-indole-2-carboxylic acid and N-benzylhydrazinecarbothioamide using General Method B. ¹H NMR (400 MHz, DMSO-d₆) δ 12.20 (s, 1H, indole NH), 8.44 (t, J=6.2 Hz, 1H, NHCH₂), 7.68 (d, J=2.1 Hz, 1H, Ar—H), 7.46-7.32 (m, 5H, Ar—H), 7.32-7.24 (m, 1H, Ar—H), 7.20 (dd, J=8.7, 2.1 Hz, 1H, Ar—H), 6.89 (s, 1H, Ar—H), 4.48 (d, J=6.1 Hz, 2H, NHCH₂). ¹³C NMR (101 MHz, DMSO-d₆) δ 163.35, 152.91, 138.65, 135.81, 128.48, 128.39, 127.35, 127.17, 124.51, 123.62, 123.42, 120.08, 113.48, 101.95, 46.13. HRESI-MS m/z calcd for [M+H]⁺ C₁₇H₁₄ClN₄O: 325.0851, found: 325.0839. mp 245-247° C.

### Example 9: N-Benzyl-5-(5-chloro-3-methyl-1H-indol-2-yl)-1,3,4-oxadiazol-2-amine (ABM315)

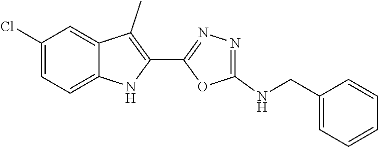 [SMILES: Cc1c(-c2nnc(NCc3ccccc3)o2)[nH]c2ccc(Cl)cc12]

N-Benzyl-5-(5-chloro-3-methyl-1H-indol-2-yl)-1,3,4-oxadiazol-2-amine was prepared as a white solid from 5-chloro-3-methyl-1H-indole-2-carboxylic acid and N-benzylhydrazinecarbothioamide using General Method B. ¹H NMR (400 MHz, DMSO-d₆) δ 11.78 (s, 1H, indole NH), 8.39 (t, J=6.2 Hz, 1H, NHCH₂), 7.64 (d, J=2.2 Hz, 1H, Ar—H), 7.45-7.32 (m, 5H, Ar—H), 7.32-7.23 (m, 1H, Ar—H), 7.19 (dd, J=8.7, 2.2 Hz, 1H, Ar—H), 4.49 (d, J=6.1 Hz, 2H, NHCH₂), 2.47 (s, 3H, CH₃). ¹³C NMR (101 MHz, DMSO-d₆) δ 163.17, 153.41, 138.76, 135.11, 129.23, 128.38, 127.36, 127.16, 123.94, 123.50, 120.21, 118.56, 113.32, 111.88, 46.20, 9.19. HRESI-MS m/z calcd for [M+H]⁺ C₁₈H₁₆ClN₄O: 339.1007, found: 339.0996. mp 220-222° C.

### Example 10: N-Benzyl-5-(5-bromo-3-ethyl-1H-indol-2-yl)-1,3,4-oxadiazol-2-amine (ABM316)

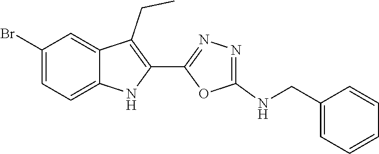 [SMILES: CCc1c(-c2nnc(NCc3ccccc3)o2)[nH]c2ccc(Br)cc12]

N-Benzyl-5-(5-bromo-3-ethyl-1H-indol-2-yl)-1,3,4-oxadiazol-2-amine was prepared as a white solid from 5-bromo-3-ethyl-1H-indole-2-carboxylic acid and N-benzylhydrazinecarbothioamide using General Method B. ¹H NMR (400 MHz, DMSO-d₆) δ 11.78 (s, 1H, indole NH), 8.41 (t, J=6.2 Hz, 1H, NHCH₂), 7.80 (d, J=1.9 Hz, 1H, Ar—H), 7.44-7.32 (m, 5H, Ar—H), 7.32-7.23 (m, 2H, Ar—H), 4.48 (d, J=6.1 Hz, 2H, NHCH₂), 2.96 (q, J=7.5 Hz, 2H, CH₂H₃), 1.17 (t, J=7.4 Hz, 3H, CH₂CH₃). ¹³C NMR (101 MHz, DMSO-d₆) δ 163.22, 153.18, 138.75, 135.41, 128.96, 128.39, 127.34, 127.17, 125.96, 121.48, 119.32, 118.58, 113.87, 111.86, 46.19, 17.06, 15.47. HRESI-MS m/z calcd for [M+H]⁺ C₁₉H₁₈BrN₄O: 397.0659, found: 397.0647. mp 218-220° C.

### Example 11: N-Phenethylhydrazinecarbothioamide

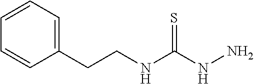 [SMILES: NNC(=S)NCCc1ccccc1]

N-Phenethylhydrazinecarbothioamide was prepared as (2-isothiocyanatoethyl)benzene from and hydrazine monohydrate using General Method A. ¹H NMR (400 MHz, DMSO-d₆) δ 8.66 (s, 1H, CH₂NH), 7.87 (s, 1H, NHNH₂), 7.34-7.15 (m, 5H), 4.45 (s, 2H, NHNH₂), 3.69 (q, J=7.1 Hz, 2H, CH₂CH₂NH), 2.83 (t, J=7.6 Hz, 2H, CH₂CH₂NH). ¹³C NMR (101 MHz, DMSO-d₆) δ 181.12, 139.40, 128.60, 128.40, 126.11, 44.44, 35.23.

### Example 12: 5-(5-Chloro-3-ethyl-1H-indol-2-yl)-N-phenethyl-1,3,4-oxadiazol-2-amine (ABM317)

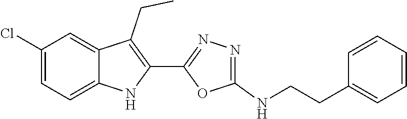 [SMILES: CCc1c(-c2nnc(NCCc3ccccc3)o2)[nH]c2ccc(Cl)cc12]

5-(5-Chloro-3-ethyl-1H-indol-2-yl)-N-phenethyl-1,3,4-oxadiazol-2-amine was prepared as a white solid from 5-chloro-3-ethyl-1H-indole-2-carboxylic acid and N-phenethylhydrazinecarbothioamide using General Method B. ¹H NMR (400 MHz, DMSO-d₆) δ 11.77 (s, 1H, indole NH), 7.94 (t, J=5.7 Hz, 1H, NHCH₂CH₂), 7.67 (d, J=2.1 Hz, 1H, Ar—H), 7.40 (d, J=8.6 Hz, 1H, Ar—H), 7.35-7.25 (m, 4H, Ar—H), 7.22-7.17 (m, 2H, Ar—H), 3.51 (q, J=6.8 Hz, 2H, NHCH₂CH₂), 3.02-2.91 (m, 4H, CH₂H₃, NHCH₂CH₂), 1.20 (t, J=7.4 Hz, 3H, CH₂CH₃). ¹³C NMR (101 MHz, DMSO-d₆) δ 163.05, 153.03, 139.05, 135.17, 128.73, 128.36, 128.28, 126.21, 123.96, 123.42, 119.59, 118.58, 118.43, 113.43, 44.22, 34.88, 17.11, 15.46. HRESI-MS m/z calcd for [M+H]⁺ C₂₀H₂₀ClN₄O: 367.1320, found: 367.1309. mp 175-177° C.

### Example 13: 5-(5-Chloro-1H-indol-2-yl)-N-phenethyl-1,3,4-oxadiazol-2-amine (ABM318)

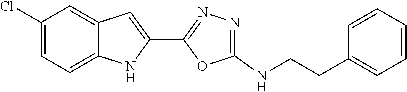 [SMILES: Clc1ccc2[nH]c(-c3nnc(NCCc4ccccc4)o3)cc2c1]

5-(5-Chloro-1H-indol-2-yl)-N-phenethyl-1,3,4-oxadiazol-2-amine was prepared as a white solid from 5-chloro-1H-indole-2-carboxylic acid and N-phenethylhydrazinecarbothioamide using General Method B. ¹H NMR (400 MHz, DMSO-d₆) δ 12.19 (s, 1H, indole NH), 7.99 (t, J=5.7 Hz, 1H, NHCH₂CH₂), 7.67 (d, J=2.1 Hz, 1H, Ar—H), 7.43 (d, J=8.7 Hz, 1H, Ar—H), 7.35-7.24 (m, 4H), 7.24-7.16 (m, 2H, Ar—H), 6.88 (d, J=2.1 Hz, 1H, Ar—H), 3.50 (q, J=7.1 Hz, 2H, NHCH₂CH₂), 2.92 (t, J=7.3 Hz, 2H, NHCH₂CH₂). ¹³C NMR (101 MHz, DMSO-d₆) δ 163.17, 152.73, 139.04, 135.79, 128.75, 128.50, 128.35, 126.21, 124.51, 123.70, 123.38, 120.06, 113.47, 101.86, 44.14, 34.76. HRESI-MS m/z calcd for [M+H]⁺ C₁₈H₁₆ClN₄O: 339.1007, found: 339.0997. mp 235-237° C.

### Example 14: 5-(5-Chloro-3-methyl-1H-indol-2-yl)-N-phenethyl-1,3,4-oxadiazol-2-amine (ABM319)

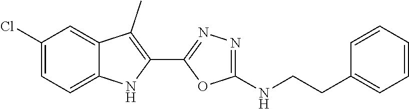 [SMILES: Cc1c(-c2nnc(NCCc3ccccc3)o2)[nH]c2ccc(Cl)cc12]

5-(5-Chloro-3-methyl-1H-indol-2-yl)-N-phenethyl-1,3,4-oxadiazol-2-amine was prepared as a white solid from 5-chloro-3-methyl-1H-indole-2-carboxylic acid and N-phenethylhydrazinecarbothioamide using General Method B. ¹H NMR (400 MHz, DMSO-d₆) δ 11.76 (s, 1H, indole NH), 7.92 (t, J=5.6 Hz, 1H, NHCH₂CH₂), 7.65 (d, J=2.0 Hz, 1H, Ar—H), 7.39 (d, J=8.7 Hz, 1H, Ar—H), 7.35-7.25 (m, 4H, Ar—H), 7.23-7.17 (m, 2H, Ar—H), 3.50 (q, J=6.8 Hz, 2H, NHCH₂CH₂), 2.93 (t, J=7.4 Hz, 2H, NHCH₂CH₂), 2.48 (s, 3H, CH₃). ¹³C NMR (101 MHz, DMSO-d₆) δ 162.99, 153.21, 139.06, 135.08, 129.24, 128.74, 128.36, 126.21, 123.93, 123.46, 120.28, 118.54, 113.30, 111.76, 44.20, 34.86, 9.22. HRESI-MS m/z calcd for [M+H]⁺ C₁₉H₁₈ClN₄O: 353.1164, found: 353.1154. mp 195-197° C.

### Example 15: 5-(5-Bromo-3-ethyl-1H-indol-2-yl)-N-phenethyl-1,3,4-oxadiazol-2-amine (ABM320)

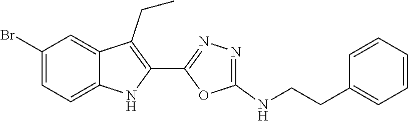 [SMILES: CCc1c(-c2nnc(NCCc3ccccc3)o2)[nH]c2ccc(Br)cc12]

5-(5-Bromo-3-ethyl-1H-indol-2-yl)-N-phenethyl-1,3,4-oxadiazol-2-amine was prepared as a white solid from 5-bromo-3-ethyl-1H-indole-2-carboxylic acid and N-phenethylhydrazinecarbothioamide using General Method B. ¹H NMR (400 MHz, DMSO-d₆) δ 11.78 (s, 1H, indole NH), 7.94 (t, J=5.7 Hz, 1H, NHCH₂CH₂), 7.81 (d, J=1.8 Hz, 1H, Ar—H), 7.40-7.17 (m, 7H, Ar—H), 3.51 (q, J=6.8 Hz, 2H, NHCH₂CH₂), 3.02-2.91 (m, 4H, CH₂CH₃, NHCH₂CH₂), 1.20 (t, J=7.4 Hz, 3H, CH₂CH₃). ¹³C NMR (101 MHz, DMSO-d₆) δ 163.06, 153.00, 139.05, 135.39, 128.98, 128.73, 128.36, 126.21, 125.92, 121.46, 119.42, 118.47, 113.86, 111.87, 44.22, 34.88, 17.10, 15.48. HRESI-MS m/z calcd for [M+H]⁺ C₂₀H₂₀BrN₄O: 411.0815, found: 411.0804. mp 170-172° C.

### Example 16: N-(4-Fluorophenyl)hydrazinecarbothioamide

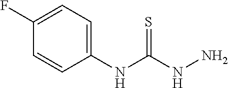 [SMILES: NNC(=S)Nc1ccc(F)cc1]

N-(4-Fluorophenyl)hydrazinecarbothioamide was prepared as a white solid from 1-fluoro-4-isothiocyanatobenzene and hydrazine monohydrate using General Method A ¹H NMR (400 MHz, DMSO-d₆) δ 9.60 (s, 1H, NHCSNHNH₂), 9.12 (s, 1H, NHCSNHNH₂), 7.64-7.56 (m, 2H, Ar—H), 7.17-7.06 (m, 2H, Ar—H), 4.77 (s, 2H, NHCSNHNH₂).

### Example 17: 5-(5-Chloro-3-ethyl-1H-indol-2-yl)-N-(4-fluorophenyl)-1,3,4-oxadiazol-2-amine (ABM335)

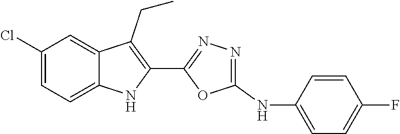 [SMILES: CCc1c(-c2nnc(Nc3ccc(F)cc3)o2)[nH]c2ccc(Cl)cc12]

5-(5-Chloro-3-ethyl-1H-indol-2-yl)-N-(4-fluorophenyl)-1,3,4-oxadiazol-2-amine was prepared as a white solid from 5-chloro-3-ethyl-1H-indole-2-carboxylic acid and N-(4-fluorophenyl)hydrazinecarbothioamide using General Method B. ¹H NMR (400 MHz, DMSO-d₆) δ 11.89 (s, 1H, indole NH), 10.68 (s, 1H, NH), 7.71 (s, J=2.0 Hz, 1H, Ar—H), 7.69-7.62 (m, 2H, Ar—H), 7.42 (d, J=8.7 Hz, 1H, Ar—H), 7.28-7.17 (m, 3H, Ar—H), 3.04 (q, J=7.4 Hz, 2H, CH₂CH₃), 1.24 (t, J=7.4 Hz, 3H, CH₂CH₃). HRESI-MS m/z calcd for [M+H]⁺ C₁₈H₁₅ClFN₄O: 357.0913, found: 357.0904. mp 248-250° C.

### Example 18: 5-(5-Chloro-1H-indol-2-yl)-N-(4-fluorophenyl)-1,3,4-oxadiazol-2-amine (ABM336)

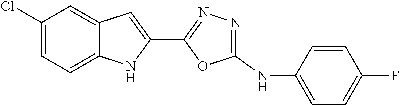 [SMILES: Fc1ccc(Nc2nnc(-c3cc4cc(Cl)ccc4[nH]3)o2)cc1]

5-(5-Chloro-1H-indol-2-yl)-N-(4-fluorophenyl)-1,3,4-oxadiazol-2-amine was prepared as a white solid from 5-chloro-1H-indole-2-carboxylic acid and N-(4-fluorophenyl)hydrazinecarbothioamide using General Method B. ¹H NMR (400 MHz, DMSO-d₆) δ 12.34 (s, 1H, indole NH), 10.78 (s, 1H, NH), 7.71 (d, J=2.1 Hz, 1H, Ar—H), 7.67-7.63 (m, 2H, Ar—H), 7.46 (d, J=8.7 Hz, 1H, Ar—H), 7.25-7.20 (m, 3H, Ar—H), 6.97 (d, J=2.0 Hz, 1H, Ar—H). HRESI-MS m/z calcd for [M+H]⁺ C₁₆H₁₁ClFN₄O: 329.0600, found: 329.0593. mp 265-267° C.

### Example 19: 5-(5-Chloro-3-methyl-1H-indol-2-yl)-N-(4-fluorophenyl)-1,3,4-oxadiazol-2-amine (ABM337)

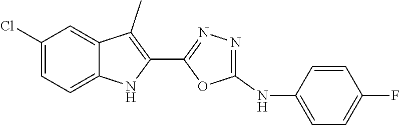 [SMILES: Cc1c(-c2nnc(Nc3ccc(F)cc3)o2)[nH]c2ccc(Cl)cc12]

5-(5-Chloro-3-methyl-1H-indol-2-yl)-N-(4-fluorophenyl)-1,3,4-oxadiazol-2-amine was prepared as a white solid from 5-chloro-3-methyl-1H-indole-2-carboxylic acid and N-(4-fluorophenyl)hydrazinecarbothioamide using General Method B. ¹H NMR (400 MHz, DMSO-d₆) δ 11.90 (s, 1H, indole NH), 10.69 (s, 1H, NH), 7.71-7.62 (m, 3H, Ar—H), 7.42 (d, J=8.6 Hz, 1H, Ar—H), 7.25-7.20 (m, 3H, Ar—H), 2.54 (s, 3H, CH₃). HRESI-MS m/z calcd for [M+H]⁺ C₁₇H₁₃ClFN₄O: 343.0756, found: 343.0748. mp 256-258° C.

### Example 20: 5-(5-Bromo-3-ethyl-1H-indol-2-yl)-N-(4-fluorophenyl)-1,3,4-oxadiazol-2-amine (ABM338)

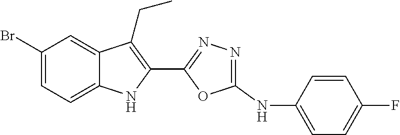 [SMILES: CCc1c(-c2nnc(Nc3ccc(F)cc3)o2)[nH]c2ccc(Br)cc12]

5-(5-Bromo-3-ethyl-1H-indol-2-yl)-N-(4-fluorophenyl)-1,3,4-oxadiazol-2-amine was prepared as a white solid from 5-bromo-3-ethyl-1H-indole-2-carboxylic acid and N-(4-fluorophenyl)hydrazinecarbothioamide using General Method B. ¹H NMR (400 MHz, DMSO-d₆) δ 11.91 (s, 1H, indole NH), 10.69 (s, 1H, NH), 7.84 (d, J=1.8 Hz, 1H, Ar—H), 7.71-7.62 (m, 2H, Ar—H), 7.39 (d, J=8.7 Hz, 1H, Ar—H), 7.32 (dd, J=8.6, 1.9 Hz, 1H, Ar—H), 7.23 (t, J=8.8 Hz, 2H, Ar—H), 3.04 (q, J=7.4 Hz, 2H, CH₂H₃), 1.24 (t, J=7.4 Hz, 3H, CH₂CH₃). HRESI-MS m/z calcd for [M+H]⁺ C₁₈H₁₅BrFN₄O: 401.0408, found: 401.0397. mp 243-245° C.

### Example 21: 5-Chloro-3-methyl-1H-indole-2-carbohydrazide

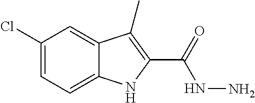

5-Chloro-3-methyl-1H-indole-2-carbohydrazide was prepared as a white solid from ethyl 5-chloro-3-methyl-1H-indole-2-carboxylate and hydrazine monohydrate using General Method C. ¹H NMR (400 MHz, DMSO-d₆) δ 11.31 (s, 1H, indole NH), 9.19 (s, 1H, NHNH₂), 7.61 (d, J=2.1 Hz, 1H, Ar—H), 7.36 (d, J=8.7 Hz, 1H, Ar—H), 7.16 (dd, J=8.6, 2.1 Hz, 1H, Ar—H), 4.52 (s, 2H, NH₂), 2.42 (s, 3H, CH₃). ¹³C NMR (101 MHz, DMSO-d₆) δ 161.97, 133.81, 129.06, 128.39, 123.64, 123.50, 118.87, 113.48, 112.55, 9.50. HRESI-MS m/z calcd for [M+H]⁺ C₁₀H₁₁ClN₃O: 224.0585, found: 224.0585. mp 241-242° C.

### Example 22: 5-(5-Chloro-3-methyl-1H-indol-2-yl)-1,3,4-oxadiazole-2-thiol

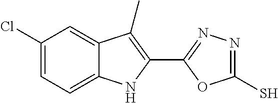 [SMILES: Cc1c(-c2nnc(S)o2)[nH]c2ccc(Cl)cc12]

5-(5-Chloro-3-methyl-1H-indol-2-yl)-1,3,4-oxadiazole-2-thiol was prepared as a white solid from 5-chloro-3-methyl-1H-indole-2-carbohydrazide and carbon disulfide using General Method D. ¹H NMR (400 MHz, DMSO-d₆) δ 12.08 (s, 1H, indole NH), 7.73 (d, J=2.0 Hz, 1H, Ar—H), 7.41 (d, J=8.7 Hz, 1H, Ar—H), 7.25 (dd, J=8.8, 2.1 Hz, 1H, Ar—H), 2.50 (s, 3H, CH₃). ¹³C NMR (101 MHz, DMSO-d₆) δ 176.44, 156.20, 135.45, 128.83, 124.64, 124.40, 119.09, 118.21, 115.12, 113.73, 9.39. HRESI-MS m/z calcd for [M+H]⁺ C₁₁H₉ClN₃OS: 266.0149, found: 266.0150. mp 194-196° C.

### Example 23: 2-(5-Chloro-3-methyl-1H-indol-2-yl)-5-((4-methoxybenzyl)thio)-1,3,4-oxadiazole (ABM301)

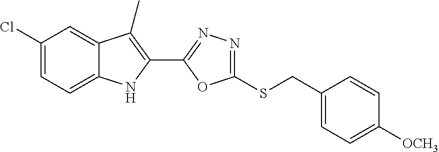 [SMILES: COc1ccc(CSc2nnc(-c3[nH]c4ccc(Cl)cc4c3C)o2)cc1]

2-(5-Chloro-3-methyl-1H-indol-2-yl)-5-((4-methoxybenzyl)thio)-1,3,4-oxadiazole was prepared as a white solid from 5-chloro-3-methyl-1H-indole-2-carbohydrazide and 1-(bromomethyl)-4-methoxybenzene using General Method E. ¹H NMR (400 MHz, DMSO-d₆) δ 12.04 (s, 1H, indole NH), 7.69 (d, J=2.0 Hz, 1H, Ar—H), 7.42-7.37 (m, 3H, Ar—H), 7.22 (dd, J=8.8, 2.1 Hz, 1H, Ar—H), 6.88 (d, J=8.6 Hz, 2H, Ar—H), 4.54 (s, 2H, SCH₂), 3.70 (s, 3H, OCH₃), 2.49 (s, 3H, CH₃). ¹³C NMR (101 MHz, DMSO-d₆) δ 162.28, 160.64, 158.89, 135.48, 130.32, 128.96, 128.07, 124.46, 124.30, 119.06, 118.90, 114.77, 114.03, 113.68, 55.09, 35.86, 9.42. HRESI-MS m/z calcd for [M+H]⁺ C₁₉H₁₇ClN₃O₂S: 386.0725, found: 386.0722. mp 210-212° C.

### Example 24: 5-Bromo-3-ethyl-1H-indole-2-carbohydrazide

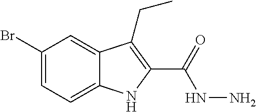 [SMILES: CCc1c(C(=O)NN)[nH]c2ccc(Br)cc12]

5-Bromo-3-ethyl-1H-indole-2-carbohydrazide was prepared as a white solid from 5-bromo-3-ethyl-1H-indole-2-carboxylic acid and hydrazine monohydrate using General Method C. ¹H NMR (400 MHz, DMSO-d₆) δ 11.29 (s, 1H, indole NH), 9.26 (s, 1H, NHNH₂), 7.80 (d, J=1.8 Hz, 1H, Ar—H), 7.36 (d, J=8.7 Hz, 1H, Ar—H), 7.29 (dd, J=8.7, 1.8 Hz, 1H, Ar—H), 4.54 (s, 2H, NH₂), 2.99 (q, J=7.4 Hz, 2H, CH₂CH₃), 1.16 (t, J=7.4 Hz, 3H, CH₂CH₃). ¹³C NMR (101 MHz, DMSO-d₆) δ 161.76, 134.06, 128.80, 127.30, 125.99, 121.84, 120.07, 114.03, 111.60, 17.08, 15.67. HRESI-MS m/z calcd for [M+H]⁺ C₁₁H₁₃BrN₃O: 282.0237, found: 282.0239. mp 221-222° C.

### Example 25: 5-(5-Bromo-3-ethyl-1H-indol-2-yl)-1,3,4-oxadiazole-2-thiol

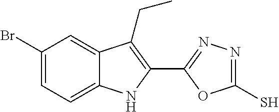 [SMILES: CCc1c(-c2nnc(S)o2)[nH]c2ccc(Br)cc12]

5-(5-Bromo-3-ethyl-1H-indol-2-yl)-1,3,4-oxadiazole-2-thiol was prepared as a white solid from 5-bromo-3-ethyl-1H-indole-2-carbohydrazide and carbon disulfide using General Method D. ¹H NMR (400 MHz, DMSO-d₆) δ 12.10 (s, 1H, indole NH), 7.88 (d, J=1.5 Hz, 1H, Ar—H), 7.36 (d, J=2.0 Hz, 2H, Ar—H), 2.99 (q, J=7.4 Hz, 2H, CH₂CH₃), 1.19 (t, J=7.4 Hz, 3H, CH₂CH₃). ¹³C NMR (101 MHz, DMSO-d₆) δ 176.47, 155.93, 135.76, 128.57, 127.08, 122.03, 121.66, 117.33, 114.25, 112.34, 17.15, 15.03. HRESI-MS m/z calcd for [M+H]⁺ C₁₂H₁₁BrN₃OS: 323.9801, found: 323.9796. mp 254-256° C.

### Example 26: 2-(Benzylthio)-5-(5-bromo-3-ethyl-1H-indol-2-yl)-1,3,4-oxadiazole (ABM305)

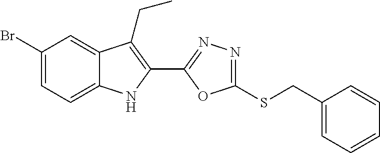 [SMILES: CCc1c(-c2nnc(SCc3ccccc3)o2)[nH]c2ccc(Br)cc12]

2-(Benzylthio)-5-(5-bromo-3-ethyl-1H-indol-2-yl)-1,3,4-oxadiazole was prepared as a white solid from 5-(5-bromo-3-ethyl-1H-indol-2-yl)-1,3,4-oxadiazole-2-thiol and (bromomethyl)benzene using General Method E. ¹H NMR (400 MHz, DMSO-d₆) δ 12.06 (s, 1H, indole NH), 7.88 (s, 1H, Ar—H), 7.48 (d, J=7.6 Hz, 2H, Ar—H), 7.43-7.24 (m, 5H, Ar—H), 4.60 (s, 2H, SCH₂), 3.02 (q, J=7.5 Hz, 2H, CH₂CH₃), 1.19 (t, J=7.4 Hz, 3H, CH₂CH₃). ¹³C NMR (101 MHz, DMSO-d₆) b 162.27, 160.45, 136.38, 135.78, 128.97, 128.70, 128.63, 127.81, 126.92, 122.01, 121.38, 117.98, 114.20, 112.25, 36.14, 17.23, 15.19. HRESI-MS m/z calcd for [M+H]⁺ C₁₉H₁₇BrN₃OS: 414.0270, found: 414.0271. mp 183-185° C.

### Example 27: 2-(5-Bromo-3-ethyl-1H-indol-2-yl)-5-((4-methoxybenzyl)thio)-1,3,4-oxadiazole (ABM309)

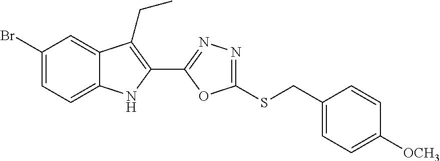 [SMILES: CCc1c(-c2nnc(SCc3ccc(OC)cc3)o2)[nH]c2ccc(Br)cc12]

2-(5-Bromo-3-ethyl-1H-indol-2-yl)-5-((4-methoxybenzyl)thio)-1,3,4-oxadiazole was prepared as a white solid from 5-(5-bromo-3-ethyl-1H-indol-2-yl)-1,3,4-oxadiazole-2-thiol and 1-(bromomethyl)-4-methoxybenzene using General Method E. ¹H NMR (400 MHz, DMSO-d₆) δ 12.08 (s, 1H, indole NH), 7.89 (s, 1H, Ar—H), 7.43-7.32 (m, 4H, Ar—H), 6.90 (d, J=8.2 Hz, 2H, Ar—H), 4.56 (s, 2H, SCH₂), 3.72 (s, 3H, OCH₃), 3.03 (q, J=7.5 Hz, 2H, CH₂CH₃), 1.19 (t, J=7.4 Hz, 3H, CH₂CH₃). ¹³C NMR (101 MHz, DMSO-d₆) δ 162.35, 160.40, 158.88, 135.78, 130.31, 128.71, 128.04, 126.92, 122.01, 121.35, 118.01, 114.22, 114.03, 112.24, 55.09, 35.86, 17.23, 15.18. HRESI-MS m/z calcd for [M+H]⁺ C₂₀H₁₉BrN₃O₂S: 444.0376, found: 444.0374. mp 181-183° C.

### Example 28: 2-(Benzylthio)-5-(5-chloro-3-methyl-1H-indol-2-yl)-1,3,4-oxadiazole (ABM321)

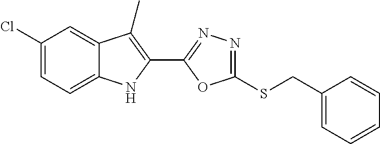 [SMILES: Cc1c(-c2nnc(SCc3ccccc3)o2)[nH]c2ccc(Cl)cc12]

2-(Benzylthio)-5-(5-chloro-3-methyl-1H-indol-2-yl)-1,3,4-oxadiazole was prepared as a white solid from 5-(5-chloro-3-methyl-1H-indol-2-yl)-1,3,4-oxadiazole-2-thiol and (bromomethyl)benzene using General Method E. ¹H NMR (400 MHz, DMSO-d₆) δ 12.05 (s, 1H, indole NH), 7.71 (d, J=2.0 Hz, 1H, Ar—H), 7.47 (d, J=7.4 Hz, 2H, Ar—H), 7.42 (d, J=8.7 Hz, 1H, Ar—H), 7.36-7.21 (m, 4H, Ar—H), 4.59 (s, 2H, CH₂), 2.50 (s, 3H, CH₃). ¹³C NMR (101 MHz, DMSO-d₆) b 162.20, 160.72, 136.47, 135.52, 129.01, 128.97, 128.65, 127.83, 124.48, 124.31, 119.08, 118.91, 114.81, 113.73, 36.15, 9.44. HRESI-MS m/z calcd for [M+H]⁺ C₁₈H₁₅ClN₃O S: 356.0619, found: 356.0627. mp 215-216° C.

### Example 29: 2-(5-Chloro-3-methyl-1H-indol-2-yl)-5-(phenethylthio)-1,3,4-oxadiazole (ABM322)

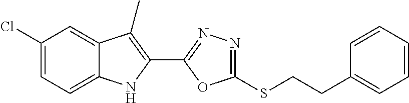 [SMILES: Cc1c(-c2nnc(SCCc3ccccc3)o2)[nH]c2ccc(Cl)cc12]

2-(5-Chloro-3-methyl-1H-indol-2-yl)-5-(phenethylthio)-1,3,4-oxadiazole was prepared as a white solid from 5-(5-chloro-3-methyl-1H-indol-2-yl)-1,3,4-oxadiazole-2-thiol and (2-bromoethyl)benzene using General Method E. ¹H NMR (400 MHz, DMSO-d₆) δ 12.04 (s, 1H, indole NH), 7.73 (d, J=1.9 Hz, 1H, Ar—H), 7.43 (d, J=8.7 Hz, 1H, Ar—H), 7.35-7.17 (m, 6H, Ar—H), 3.59 (t, J=7.5 Hz, 2H, SCH₂CH₂), 3.11 (t, J=7.5 Hz, 2H, SCH₂CH₂), 2.54 (s, 3H, CH₃). ¹³C NMR (101 MHz, DMSO-d₆) δ 162.63, 160.53, 139.17, 135.46, 128.98, 128.68, 128.43, 126.57, 124.41, 124.27, 119.06, 119.03, 114.62, 113.68, 35.01, 33.42, 9.44. HRESI-MS m/z calcd for [M+H]⁺ C₁₉H₁₇ClN₃OS: 370.0775, found: 370.0776. mp 222-224° C.

### Example 30: 2-(5-Chloro-3-methyl-1H-indol-2-yl)-5-((3-phenylpropyl)thio)-1,3,4-oxadiazole (ABM323)

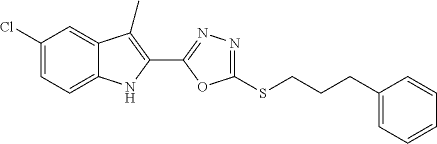 [SMILES: Cc1c(-c2nnc(SCCCc3ccccc3)o2)[nH]c2ccc(Cl)cc12]

2-(5-Chloro-3-methyl-1H-indol-2-yl)-5-((3-phenylpropyl)thio)-1,3,4-oxadiazole was prepared as a white solid from 5-(5-chloro-3-methyl-1H-indol-2-yl)-1,3,4-oxadiazole-2-thiol and (3-bromopropyl)benzene using General Method E. ¹H NMR (400 MHz, DMSO-d₆) δ 12.04 (s, 1H, indole NH), 7.70 (d, J=2.1 Hz, 1H, Ar—H), 7.41 (d, J=8.7 Hz, 1H, Ar—H), 7.31-7.12 (m, 6H, Ar—H), 3.30 (t, J=7.2 Hz, 2H, SCH₂CH₂CH₂), 2.73 (t, J=7.6 Hz, 2H, SCH₂CH₂CH₂), 2.50 (s, 3H, CH₃), 2.12-2.03 (m, 2H, SCH₂CH₂CH₂). ¹³C NMR (101 MHz, DMSO-d₆) δ 162.70, 160.52, 140.79, 135.46, 128.96, 128.38, 128.33, 125.99, 124.40, 124.26, 119.04, 118.98, 114.61, 113.68, 33.80, 31.81, 30.70, 9.40. HRESI-MS m/z calcd for [M+H]⁺ C₂₀H₁₉ClN₃OS: 384.0932, found: 384.0934. mp 179-180° C.

### Example 31: 2-(5-Chloro-3-methyl-1H-indol-2-yl)-5-((4-methylbenzyl)thio)-1,3,4-oxadiazole (ABM324)

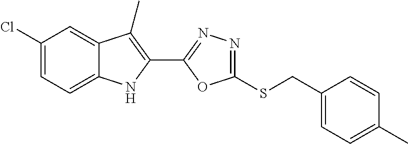 [SMILES: Cc1ccc(CSc2nnc(-c3[nH]c4ccc(Cl)cc4c3C)o2)cc1]

2-(5-Chloro-3-methyl-1H-indol-2-yl)-5-((4-methylbenzyl)thio)-1,3,4-oxadiazole was prepared as a white solid from 5-(5-chloro-3-methyl-1H-indol-2-yl)-1,3,4-oxadiazole-2-thiol and 1-(bromomethyl)-4-methylbenzene using General Method E. ¹H NMR (400 MHz, DMSO-d₆) δ 11.80 (s, 1H, indole NH), 7.67 (d, J=2.3 Hz, 1H, Ar—H), 7.41 (d, J=8.7 Hz, 1H, Ar—H), 7.32 (d, J=8.1 Hz, 2H, Ar—H), 7.20 (d, J=8.7 Hz, 1H, Ar—H), 7.10 (d, J=8.2 Hz, 2H, Ar—H), 4.52 (s, 2H, SCH₂), 2.48 (s, 3H, CH₃), 2.22 (s, 3H, CH₃). ¹³C NMR (101 MHz, DMSO-d₆) δ 162.18, 160.73, 137.13, 135.61, 133.32, 129.18, 128.98, 128.92, 124.38, 124.24, 119.03, 114.70, 113.79, 35.98, 20.71, 9.44. HRESI-MS m/z calcd for [M+H]⁺ C₁₉H₁₇ClN₃OS: 370.0775, found: 370.0763. mp 212-214° C.

### Example 32: 2-(5-Bromo-3-ethyl-1H-indol-2-yl)-5-((4-methoxybenzyl)thio)-1,3,4-oxadiazole (ABM325)

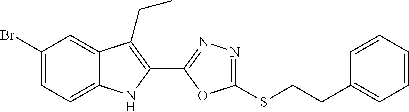 [SMILES: CCc1c(-c2nnc(SCCc3ccccc3)o2)[nH]c2ccc(Br)cc12]

2-(5-Bromo-3-ethyl-1H-indol-2-yl)-5-((4-methoxybenzyl)thio)-1,3,4-oxadiazole was prepared as a white solid from 5-(5-bromo-3-ethyl-1H-indol-2-yl)-1,3,4-oxadiazole-2-thiol and (2-bromoethyl)benzene using General Method E. ¹H NMR (400 MHz, DMSO-d₆) δ 12.07 (s, 1H, indole NH), 7.89 (s, 1H, Ar—H), 7.43-7.19 (m, 7H, Ar—H), 3.59 (t, J=7.5 Hz, 2H, SCH₂CH₂), 3.13-3.03 (m, 4H, SCH₂CH₂, CH₂CH₃), 1.21 (t, J=7.4 Hz, 3H, CH₂CH₃). ¹³C NMR (101 MHz, DMSO-d₆) b 162.68, 160.28, 139.15, 135.76, 128.72, 128.67, 128.41, 126.85, 126.56, 121.98, 121.20, 118.12, 114.21, 112.21, 35.00, 33.42, 17.26, 15.21. HRESI-MS m/z calcd for [M+H]⁺ C₂₀H₁₉BrN₃OS: 428.0427, found: 428.0428. mp 185-186° C.

### Example 33: 2-(5-Bromo-3-ethyl-1H-indol-2-yl)-5-((3-phenylpropyl)thio)-1,3,4-oxadiazole (ABM326)

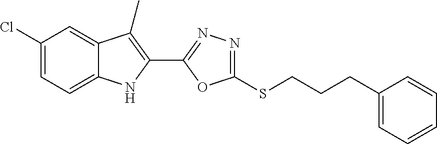 [SMILES: Cc1c(-c2nnc(SCCCc3ccccc3)o2)[nH]c2ccc(Cl)cc12]

2-(5-Bromo-3-ethyl-1H-indol-2-yl)-5-((3-phenylpropyl)thio)-1,3,4-oxadiazole was prepared as a white solid from 5-bromo-3-ethyl-1H-indole-2-carbohydrazide and (3-bromopropyl)benzene using General Method E. ¹H NMR (400 MHz, CDCl₃) δ 10.20 (s, 1H, indole NH), 7.79 (d, J=1.7 Hz, 1H, Ar—H), 7.53 (d, J=8.7 Hz, 1H, Ar—H), 7.36-7.18 (m, 6H, Ar—H), 3.31 (t, J=7.4 Hz, 2H, SCH₂CH₂CH₂), 3.04 (q, J=7.5 Hz, 2H, CH₂CH₃), 2.83 (t, J=7.5 Hz, 2H, SCH₂CH₂CH₂), 2.25-2.17 (m, 2H, SCH₂CH₂CH₂), 1.29 (t, J=7.5 Hz, 3H, CH₂CH₃). ¹³C NMR (101 MHz, CDCl₃) δ 164.13, 161.10, 140.49, 136.10, 129.40, 128.74, 128.59, 128.00, 126.45, 122.98, 122.59, 117.94, 114.03, 113.43, 34.64, 32.22, 30.88, 18.04, 15.57. HRESI-MS m/z calcd for [M+H]⁺ C₂₁H₂₁BrN₃OS: 442.0583, found: 442.0582. mp 134-135° C.

### Example 34: 2-(5-Bromo-3-ethyl-1H-indol-2-yl)-5-((4-methylbenzyl)thio)-1,3,4-oxadiazole (ABM327)

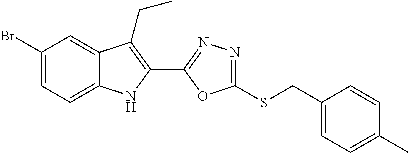 [SMILES: CCc1c(-c2nnc(SCc3ccc(C)cc3)o2)[nH]c2ccc(Br)cc12]

2-(5-Bromo-3-ethyl-1H-indol-2-yl)-5-((4-methylbenzyl)thio)-1,3,4-oxadiazole was prepared as a white solid from 5-(5-bromo-3-ethyl-1H-indol-2-yl)-1,3,4-oxadiazole-2-thiol and 1-(bromomethyl)-4-methylbenzene using General Method E. ¹H NMR (400 MHz, DMSO-d₆) δ 12.13 (s, 1H, indole NH), 7.88 (d, J=1.7 Hz, 1H, Ar—H), 7.44-7.31 (m, 4H, Ar—H), 7.14 (d, J=8.0 Hz, 2H, Ar—H), 4.56 (s, 2H, SCH₂), 3.02 (q, J=7.4 Hz, 2H, CH₂CH₃), 2.26 (s, 3H, CH₃), 1.18 (t, J=7.4 Hz, 3H, CH₂CH₃). ¹³C NMR (101 MHz, DMSO-d₆) δ 162.29, 160.42, 137.13, 135.80, 133.28, 129.18, 128.90, 128.70, 126.90, 122.00, 121.33, 117.98, 114.25, 112.23, 35.99, 20.70, 17.22, 15.18. HRESI-MS m/z calcd for [M+H]⁺ C₂₀H₁₉BrN₃OS: 428.0427, found: 428.0417. mp 201-203° C.

### Example 35: 5-Chloro-3-ethyl-1H-indole-2-carbohydrazide

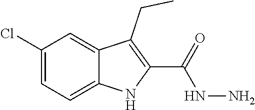 [SMILES: CCc1c(C(=O)NN)[nH]c2ccc(Cl)cc12]

5-Chloro-3-ethyl-1H-indole-2-carbohydrazide was prepared as a white solid from ethyl 5-chloro-3-ethyl-1H-indole-2-carboxylate and hydrazine monohydrate using General Method C. ¹H NMR (400 MHz, DMSO-d₆) δ 11.34 (s, 1H, indole NH), 9.29 (s, 1H, NH), 7.65 (d, J=2.0 Hz, 1H, Ar—H), 7.40 (d, J=8.7 Hz, 1H, Ar—H), 7.18 (dd, J=8.7, 2.1 Hz, 1H, Ar—H), 4.59 (s, 2H, NH₂), 3.00 (q, J=7.4 Hz, 2H, CH₂CH₃), 1.16 (t, J=7.5 Hz, 3H, CH₂CH₃). HRESI-MS m/z calcd for [M+H]⁺ C₁₁H₁₃ClN₃O: 238.0742, found: 238.0736. mp 205-207° C.

### Example 36: 5-(5-Chloro-3-ethyl-1H-indol-2-yl)-1,3,4-oxadiazole-2-thiol

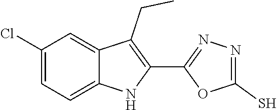 [SMILES: CCc1c(-c2nnc(S)o2)[nH]c2ccc(Cl)cc12]

5-(5-Chloro-3-ethyl-1H-indol-2-yl)-1,3,4-oxadiazole-2-thiol was prepared as a white solid from 5-chloro-3-ethyl-1H-indole-2-carbohydrazide and carbon disulfide using General Method D. ¹H NMR (400 MHz, DMSO-d₆) δ 12.09 (s, 1H, indole NH), 7.75 (s, 1H, Ar—H), 7.42 (d, J=8.8 Hz, 1H, Ar—H), 7.25 (dd, J=8.7, 1.6 Hz, 1H, Ar—H), 3.00 (q, J=7.5 Hz, 2H, CH₂CH₃), 1.20 (t, J=7.4 Hz, 3H, CH₂CH₃). HRESI-MS m/z calcd for [M+H]⁺ C₁₂H₁₁ClN₃OS: 280.0306, found: 280.0302. mp 245-247° C.

### Example 37: 2-(Benzylthio)-5-(5-chloro-3-ethyl-1H-indol-2-yl)-1,3,4-oxadiazole (ABM328)

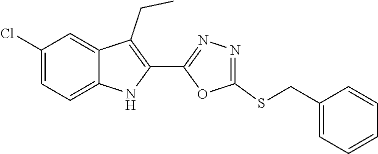 [SMILES: CCc1c(-c2nnc(SCc3ccccc3)o2)[nH]c2ccc(Cl)cc12]

2-(Benzylthio)-5-(5-chloro-3-ethyl-1H-indol-2-yl)-1,3,4-oxadiazole was prepared as a white solid from 5-(5-chloro-3-ethyl-1H-indol-2-yl)-1,3,4-oxadiazole-2-thiol and (bromomethyl)benzene using General Method E. ¹H NMR (400 MHz, DMSO-d₆) δ 12.05 (s, 1H, indole NH), 7.74 (s, 1H, Ar—H), 7.48 (d, J=7.6 Hz, 2H, Ar—H), 7.43 (d, J=8.6 Hz, 1H, Ar—H), 7.40-7.27 (m, 3H, Ar—H), 7.23 (d, J=8.8 Hz, 1H, Ar—H), 4.60 (s, 2H, SCH₂), 3.04 (q, J=7.5 Hz, 2H, CH₂CH₃), 1.19 (t, J=7.5 Hz, 3H, CH₂CH₃). HRESI-MS m/z calcd for [M+H]⁺ C₁₉H₁₇ClN₃OS: 370.0775, found: 370.0765. mp 180-182° C.

### Example 38: 2-(5-Chloro-3-ethyl-1H-indol-2-yl)-5-(phenethylthio)-1,3,4-oxadiazole (ABM329)

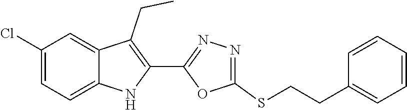 [SMILES: CCc1c(-c2nnc(SCCc3ccccc3)o2)[nH]c2ccc(Cl)cc12]

2-(5-Chloro-3-ethyl-1H-indol-2-yl)-5-(phenethylthio)-1,3,4-oxadiazole was prepared as a white solid from 5-(5-chloro-3-ethyl-1H-indol-2-yl)-1,3,4-oxadiazole-2-thiol and (2-bromoethyl)benzene using General Method E. ¹H NMR (400 MHz, DMSO-d₆) δ 12.10 (s, 1H, indole NH), 7.74 (d, J=2.0 Hz, 1H, Ar—H), 7.45 (d, J=8.7 Hz, 1H, Ar—H), 7.34-7.29 (m, 4H, Ar—H), 7.25-7.20 (m, 2H, Ar—H), 3.59 (t, J=7.5 Hz, 2H, SCH₂CH₂), 3.13-3.03 (m, 4H, SCH₂CH₂, CH₂CH₃), 1.21 (t, J=7.4 Hz, 3H, CH₂CH₃). HRESI-MS m/z calcd for [M+H]⁺ C₂₀H₁₉ClN₃OS: 384.0932, found: 384.0921.

### Example 39: 2-(5-Chloro-3-ethyl-1H-indol-2-yl)-5-((3-phenylpropyl)thio)-1,3,4-oxadiazole (ABM330)

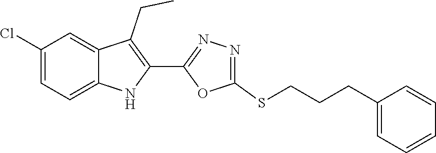 [SMILES: CCc1c(-c2nnc(SCCCc3ccccc3)o2)[nH]c2ccc(Cl)cc12]

2-(5-Chloro-3-ethyl-1H-indol-2-yl)-5-((3-phenylpropyl)thio)-1,3,4-oxadiazole was prepared as a white solid from 5-(5-chloro-3-ethyl-1H-indol-2-yl)-1,3,4-oxadiazole-2-thiol and (3-bromopropyl)benzene using General Method E. ¹H NMR (400 MHz, DMSO-d₆) δ 12.06 (s, 1H, indole NH), 7.73 (d, J=1.9 Hz, 1H, Ar—H), 7.43 (d, J=8.7 Hz, 1H, Ar—H), 7.30-7.16 (m, 6H, Ar—H), 3.32 (t, J=7.2 Hz, 2H, SCH₂CH₂CH₂), 3.04 (q, J=7.5 Hz, 2H, CH₂CH₃), 2.75 (t, J=7.6 Hz, 2H, SCH₂CH₂CH₂), 2.09 (p, J=7.5 Hz, 2H, SCH₂CH₂CH₂), 1.20 (t, J=7.4 Hz, 3H, CH₂CH₃). HRESI-MS m/z calcd for [M+H]⁺ C₂₁H₂₁ClN₃OS: 398.1088, found: 398.1077. mp 170-172° C.

### Example 40: 2-(5-Chloro-3-ethyl-1H-indol-2-yl)-5-((4-methoxybenzyl)thio)-1,3,4-oxadiazole (ABM331)

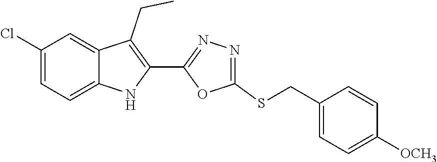 [SMILES: CCc1c(-c2nnc(SCc3ccc(OC)cc3)o2)[nH]c2ccc(Cl)cc12]

2-(5-Chloro-3-ethyl-1H-indol-2-yl)-5-((4-methoxybenzyl)thio)-1,3,4-oxadiazole was prepared as a white solid from 5-(5-chloro-3-ethyl-1H-indol-2-yl)-1,3,4-oxadiazole-2-thiol and 1-(bromomethyl)-4-methoxybenzene using General Method E. ¹H NMR (400 MHz, DMSO-d₆) δ 12.15 (s, 1H, indole NH), 7.73 (t, J=1.8 Hz, 1H, Ar—H), 7.45-7.38 (m, 3H, Ar—H), 7.23 (dd, J=8.7, 1.9 Hz, 1H, Ar—H), 6.89 (d, J=8.6 Hz, 2H, Ar—H), 4.55 (s, 2H, SCH₂), 3.72 (s, 3H, OCH₃), 3.03 (q, J=7.5 Hz, 2H, CH₂CH₃), 1.19 (t, J=7.4 Hz, 3H, CH₂CH₃). HRESI-MS m/z calcd for [M+H]⁺ C₂₀H₁₉ClN₃O₂S: 400.0881, found: 400.0870. mp 179-181° C.

### Example 41: 2-(5-Chloro-3-ethyl-1H-indol-2-yl)-5-((4-methylbenzyl)thio)-1,3,4-oxadiazole (ABM332)

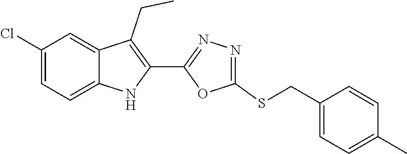 [SMILES: CCc1c(-c2nnc(SCc3ccc(C)cc3)o2)[nH]c2ccc(Cl)cc12]

2-(5-Chloro-3-ethyl-1H-indol-2-yl)-5-((4-methylbenzyl)thio)-1,3,4-oxadiazole was prepared as a white solid from 5-(5-chloro-3-ethyl-1H-indol-2-yl)-1,3,4-oxadiazole-2-thiol and 1-(bromomethyl)-4-methylbenzene using General Method E. ¹H NMR (400 MHz, DMSO-d₆) δ 12.63 (s, 1H, indole NH), 8.25 (d, J=2.0 Hz, 1H, Ar—H), 7.96 (d, J=8.6 Hz, 1H, Ar—H), 7.87 (d, J=7.7 Hz, 2H, Ar—H), 7.75 (dd, J=8.6, 2.0 Hz, 1H, Ar—H), 7.65 (d, J=7.7 Hz, 2H, Ar—H), 5.07 (s, 2H, SCH₂), 3.55 (q, J=7.5 Hz, 2H, CH₂H₃), 2.78 (s, 3H, C₆H₄CH₃), 1.71 (t, J=7.5 Hz, 3H, CH₂CH₃). HRESI-MS m/z calcd for [M+H]⁺ C₂₀H₁₉ClN₃OS: 384.0932, found: 384.0923. mp 195-197° C.

### Example 42: 1 H-Indole-2-carbohydrazide

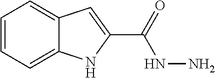 [SMILES: NNC(=O)c1cc2ccccc2[nH]1]

1H-Indole-2-carbohydrazide was prepared as a white solid from ethyl 1H-indole-2-carboxylate and hydrazine monohydrate using General Method C. ¹H NMR (400 MHz, DMSO-d₆) δ 11.62 (s, 1H, indole NH), 9.80 (s, 1H, NHNH₂), 7.59 (d, J=8.0 Hz, 1H, Ar—H), 7.45 (d, J=8.2 Hz, 1H, Ar—H), 7.17 (dd, J=8.2, 6.9 Hz, 1H, Ar—H), 7.11 (s, 1H, Ar—H), 7.03 (dd, J=8.0, 6.9 Hz, 1H, Ar—H), 4.54 (s, 2H, NH₂). ¹³C NMR (101 MHz, DMSO-d₆) δ 161.32, 136.38, 130.53, 127.18, 123.17, 121.45, 119.76, 112.32, 101.95. HRESI-MS m/z calcd for [M+H]⁺ C₉H₁₀N₃O: 176.0818, found: 176.0818. mp 230-232° C.

### Example 43: 5-(1H-Indol-2-yl)-1,3,4-oxadiazole-2-thiol

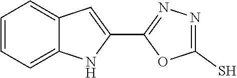 [SMILES: Sc1nnc(-c2cc3ccccc3[nH]2)o1]

5-(1H-Indol-2-yl)-1,3,4-oxadiazole-2-thiol was prepared as a white solid from 1H-indole-2-carbohydrazide and carbon disulfide using General Method D. ¹H NMR (400 MHz, DMSO-d₆) δ 12.19 (s, 1H, indole NH), 7.66 (d, J=8.2 Hz, 1H, Ar—H), 7.46 (d, J=8.3 Hz, 1H, Ar—H), 7.26 (dd, J=8.3, 7.0 Hz, 1H, Ar—H), 7.17 (d, J=2.2 Hz, 1H, Ar—H), 7.10 (dd, J=8.1, 6.9 Hz, 1H, Ar—H). ¹³C NMR (101 MHz, DMSO-d₆) δ 176.94, 156.04, 137.79, 127.12, 124.47, 121.55, 120.46, 120.19, 112.27, 105.21. mp 190-192° C.

### Example 44: 2-(Benzylthio)-5-(1H-indol-2-yl)-1,3,4-oxadiazole (ABM333)

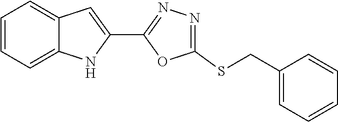 [SMILES: c1ccc(CSc2nnc(-c3cc4ccccc4[nH]3)o2)cc1]

2-(Benzylthio)-5-(1H-indol-2-yl)-1,3,4-oxadiazole was prepared as a white solid from 5-(1H-Indol-2-yl)-1,3,4-oxadiazole-2-thiol and (bromomethyl)benzene using General Method E. ¹H NMR (400 MHz, DMSO-d₆) b 12.23 (d, J=2.2 Hz, 1H, indole NH), 7.67 (d, J=8.0 Hz, 1H, Ar—H), 7.53-7.44 (m, 3H, Ar—H), 7.40-7.31 (m, 2H, Ar—H), 7.31-7.21 (m, 2H, Ar—H), 7.18 (d, J=2.2 Hz, 1H, Ar—H), 7.10 (dd, J=8.0, 7.0 Hz, 1H, Ar—H), 4.60 (s, 2H, SCH₂). ¹³C NMR (101 MHz, DMSO-d₆) δ 162.51, 160.54, 137.76, 136.45, 129.03, 128.61, 127.81, 127.23, 124.28, 121.47, 120.61, 120.37, 112.26, 105.00, 36.08. HRESI-MS m/z calcd for [M+H]⁺ C₁₇H₁₄N₃OS: 308.0852, found: 308.0850. mp 178-180° C.

### Example 45: 2-(1H-Indol-2-yl)-5-(phenethylthio)-1,3,4-oxadiazole (ABM334)

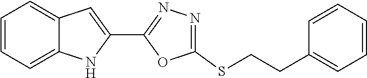 [SMILES: c1ccc(CCSc2nnc(-c3cc4ccccc4[nH]3)o2)cc1]

2-(1H-Indol-2-yl)-5-(phenethylthio)-1,3,4-oxadiazole was prepared as a white solid from 5-(1H-Indol-2-yl)-1,3,4-oxadiazole-2-thiol and (2-bromoethyl)benzene using General Method E. ¹H NMR (400 MHz, DMSO-d₆) b 11.24 (s, 1H, indole NH), 7.63 (d, J=8.0 Hz, 2H, Ar—H), 7.46 (d, J=8.3 Hz, 2H, Ar—H), 7.34-7.16 (m, 4H, Ar—H), 7.04 (t, J=7.5 Hz, 2H, Ar—H), 3.59 (t, J=7.5 Hz, 2H, SCH₂CH₂), 3.11 (t, J=7.5 Hz, 2H, SCH₂CH₂). HRESI-MS m/z calcd for [M+H]⁺ C₁₈H₁₆N₃OS: 322.1009, found: 322.1002. mp 184-186° C.

### Example 46: 2-(5-Chloro-3-methyl-1H-indol-2-yl)-5-((4-fluorobenzyl)thio)-1,3,4-oxadiazole (ABM339)

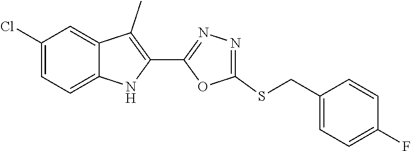 [SMILES: Cc1c(-c2nnc(SCc3ccc(F)cc3)o2)[nH]c2ccc(Cl)cc12]

2-(5-Chloro-3-methyl-1H-indol-2-yl)-5-((4-fluorobenzyl)thio)-1,3,4-oxadiazole was prepared as a white solid from 5-(5-chloro-3-methyl-1H-indol-2-yl)-1,3,4-oxadiazole-2-thiol and 1-(bromomethyl)-4-fluorobenzene using General Method E. ¹H NMR (400 MHz, DMSO-d₆) δ 11.98 (s, 1H, indole NH), 7.69 (d, J=2.2 Hz, 1H, Ar—H), 7.53 (dd, J=8.3, 2.3 Hz, 2H, Ar—H), 7.43 (dd, J=8.7, 2.3 Hz, 1H, Ar—H), 7.23 (dd, J=8.7, 2.4 Hz, 1H, Ar—H), 7.16 (dd, J=9.0, 2.5 Hz, 2H, Ar—H), 4.59 (s, 2H, SCH₂), 2.50 (s, 3H, CH₃). HRESI-MS m/z calcd for [M+H]⁺ C₁₈H₁₄ClFN₃OS: 374.0525, found: 374.0514. mp 210-212° C.

### Example 47: 2-(5-Bromo-3-ethyl-1H-indol-2-yl)-5-((4-fluorobenzyl)thio)-1,3,4-oxadiazole (ABM340)

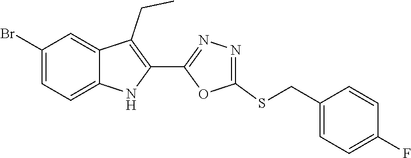 [SMILES: CCc1c(-c2nnc(SCc3ccc(F)cc3)o2)[nH]c2ccc(Br)cc12]

2-(5-Bromo-3-ethyl-1H-indol-2-yl)-5-((4-fluorobenzyl)thio)-1,3,4-oxadiazole was prepared as a white solid from 5-(5-bromo-3-ethyl-1H-indol-2-yl)-1,3,4-oxadiazole-2-thiol and 1-(bromomethyl)-4-fluorobenzene using General Method E. ¹H NMR (400 MHz, DMSO-d₆) δ 12.06 (s, 1H, indole NH), 7.86 (s, 1H, Ar—H), 7.53 (d, J=8.4 Hz, 2H, Ar—H), 7.41 (d, J=8.8 Hz, 1H, Ar—H), 7.34 (d, J=8.8 Hz, 1H, Ar—H), 7.16 (d, J=8.7 Hz, 2H, Ar—H), 4.60 (s, 2H, SCH₂), 3.01 (q, J=7.2 Hz, 2H, CH₂CH₃), 1.17 (t, J=7.2 Hz, 3H, CH₂CH₃). HRESI-MS m/z calcd for [M+H]⁺ C₁₉H₁₆BrFN₃OS: 432.0176, found: 432.0176. mp 185-187° C.

### Biological Studies

### Cannabinoid (CB) Receptor Allosteric Modulators—Functional Characteristics

Cannabinoid receptor allosteric ligands may be functionally characterised, for example, according to:

(1) their effect upon agonist binding; and/or

(2) their effect upon agonist-induced activity efficacy.

For example, allosteric interactions at the cannabinoid CB1 receptor have recently been described (see, e.g., Price et al., 2005). Three compounds, ORG27569, ORG29647 and ORG27759, produce a slowing of the dissociation of radiolabelled CB1 receptor agonist CP55940 from the CB1 receptor in mouse brain membranes. This slowing of the dissociation of the agonist is indicative of an allosteric modulator (see, e.g., Price et al., 2005). These three ORG compounds also induce an increase in the binding of CP55940 in an equilibrium binding assay and a decrease in the binding of a radiolabelled inverse agonist SR141716A. Thus, these modulators display a markedly divergent effect on orthosteric ligand affinity versus efficacy; they are allosteric enhancers of agonist binding affinity and allosteric inhibitors of agonist activity efficacy.

Irrespective of mechanism, ligands can be classified solely on their overall functional effects: ligands which amplify the effect of an agonist are known as allosteric enhancers or positive allosteric modulators; ligands which suppress the effect of an agonist are known as allosteric inhibitors or negative allosteric modulators.

### Assays Used to Investigate Allosteric Modulation

In order to fully investigate the effects of allosteric modulators, it is important to investigate the functional effects. When an allosteric ligand binds, it can either increase or decrease the dissociation rates of radiolabelled ligands. The use of functional assays to find allosteric modulators is also widely used as a screening mechanism and is required in order to define the overall modulatory effect, positive or negative. It is possible to find allosteric modulators that affect activity but don't affect affinity, which could be missed using the radioligand binding assays (equilibrium and dissociation) (see, e.g., Christopolous et al., 2004). Functional assays such as the β-arrestin recruitment and [³⁵S]GTPγS binding assays represent an excellent starting point for G-protein coupled receptor based drug discovery where the main purpose of drug discovery is to find compounds that exert functional effects.

### Functional Assays

Functional assays are used to find allosteric modulators, and are also widely used as a screening mechanism and are required in order to define the overall modulatory effect, positive or negative. It is possible to identify allosteric modulators that affect activity but don't affect affinity, and which could be missed using only a radioligand binding assay (equilibrium and dissociation) (see, e.g., Christopolous et al., 2004).

Negative allosteric modulators will cause a characteristic decrease in efficacy (Eₘₐₓ) of an orthosteric agonist in a functional assay. In contrast, competitive antagonists cause no change in agonist efficacy, but do cause a decrease in the potency (EC₅₀). Positive allosteric modulators will show a characteristic increase in the efficacy (Eₘₐₓ) of the orthosteric agonist.

### PathHunter™ β-Arrestin Assays

β-Arrestins are multifunctional intracellular proteins that interact with a structurally diverse group of cell surface receptors including GPCRs, to regulate cellular functions (see, e.g., Violin et al., 2007).

PathHunter™ enzyme fragment complementation is the most important method to measure β-arrestin recruitment. PathHunter™ β-Arrestin assays developed by DiscoveRX are revolutionary high-throughput screening assays for monitoring GPCR activation following ligand stimulation, without an imaging instrument, fluorescent protein tag, or radioactivity.

Instead, the assays detect GPCR activation through binding of β-arrestin to the expressed GPCR of interest, and measure the interaction of the two proteins using enzyme fragment complementation (EFC). The EFC approach offers a range of benefits for screening, including signal amplification and robust performance.

In this assay, the β-galactosidase enzyme (β-gal) is split into two inactive fragments. The larger portion of β-gal, termed EA for enzyme acceptor, is fused to the C-terminus of β-arrestin. The smaller, complementing fragment of β-gal, the ProLink™ tag, is expressed as a fusion protein with the GPCR of interest at the C-terminus. Upon activation, the GPCR is bound by β-arrestin. The interaction of β-arrestin and the GPCR forces the interaction of ProLink and EA, thus allowing complementation of the two fragments of β-gal and the formation of a functional enzyme capable of hydrolyzing substrate and generating a fluorescence signal.

Using the PathHunter™ CB1 kit from DiscoveRX, a cannabinoid CB1 receptor agonist is added and activates the receptor, EA, which is fused to the C-terminus of β-arrestin, and then interacts with the ProLink™ tag which is fused to the CB1 receptor. This will result in the fluorescence signal that is recorded by a luminescence plate reader. The fluorescence signal is directly related to the activation of the receptor, therefore a higher concentration of agonist will yield a larger fluorescence signal. The cannabinoid agonist is added in increasing concentrations to obtain an agonist dose response curve. With pre-incubation of a potential allosteric modulator, either an enhancement of agonist activity with an allosteric enhancer or a decrease in the maximal response of the agonist with an allosteric inhibitor is expected.

All raw data are obtained as luminescence (relative light units), and are normalised and presented as a percentage of the maximal response for the cannabinoid CB1 receptor agonist CP55,940 (2-[(1R,2R,5R)-5-hydroxy-2-(3-hydroxypropyl)cyclohexyl]-5-(2-methyloctan-2-yl)phenol).

### Animal Models of BD and SCZ (Dopaminergic and Glutamatergic Dysfunction)

### Dopamine Transporter Knockout Mice (DATKO):

DAT1/SLC6A3 gene polymorphism associations have been identified in various psychiatric disorders, including ADHD, BD and SCZ. Also, in line with reduced functioning of the dopamine transporter (DAT) in BD patients, mice without functioning DAT display behavioural abnormalities that model mania observed in BD and that mimic positive symptoms in SCZ. These mice are accepted as a model for assessing the effects of novel small molecules that may have utility in modulating the dopamine dysfunction; deficits respond to both lithium and antipsychotics (Beaulieu et al., 2004; Gainetdinov et al., 1999; Ralph et al., 2001).

### GluN1 Knockdown Mice (GluN1KD):

Functional NMDARs are composed of a common GluN1 subunit and one of four GluN2 subunits (GluN2A-GluN2D) combined in an undetermined ratio to make the heteromeric receptor complex. Recent studies using post mortem prefrontal cortex samples from patients with SCZ and depression, display a series of changes in NMDAR regulation. Endocannabinoids, via CB₁Rs, control the presence of NR1 C1 subunits in the neural membrane which has implications for psychosis and SCZ. The data demonstrate that the activity of NMDARs requires control via CB1Rs and disproportionate endocannabinoid control could cause NMDAR hypofunction (Rodriguez-MuAoz et al., 2017; Rodriguez-MuAoz et al., 2016). The GluN1KD (NMDAR hypofunction model) mouse line expresses 5-10% of the normal level of the subunit. GluN1KD mice display behavioural abnormalities that have been related to SCZ; these behaviours are normalized by the antipsychotics haloperidol and clozapine. At the dopamine level, GluN1KD mice have remodelled dopamine neurons, leading to a state of hyperdopaminergia, manifesting as an increase in tonic firing rates of dopaminergic neurons (Ferris et al., 2014). The GluN1KD mouse model displays both construct (Demontis et al., 2011) as well as predictive validity (Mohn et al., 1999) these mice are accepted as a model for assessing the effects of novel small modulators that may have utility in treating the positive and negative symptoms of SCZ.

| | GluN1KD | DATKO |
|---|---|---|
| Construct | De novo mutations found in | De novo mutations found in |
| Validity | both GluN1 and GluN2 | both GluN1 and GluN2 |
| | leading to ID and SCZ | leading to ID and SCZ |
| Face | Mimic behaviorual | Mimic behavioural |
| Validity | abnormalities of SCZ | abnormalities to BD and |
| | Hyperlocomotion, | positive symptoms of SCZ |
| | increased stereotypy, | Hyperlocomotion, |
| | disrupted sensorimotor | disrupted sensorimotor |
| | gating, increased mania- | gating and increased |
| | like behaviours | impulsivity |
| Predictive | Dysregulation of these | Hyperactivity and |
| Validity | behaviours have been | sensorimotor deficits both |
| | shown to be normalized by | respond to lithium and |
| | the antipsychotic drugs | classic antipsychotics |
| | haloperidol and clozapine | |

### Behavioural Assay Methods:

Adult (>P70) GluN1-knockdown (GluN1KD—F1: C571Bl/6J x 129S1/SvlmJ) and DAT-knockout (DATKO—C57Bl/6J), balanced for sex, were treated with either vehicle (1:1:18-Tween80:95% ethanol:saline) or a novel CB1 negative allosteric modulator (CB1 NAM), ABM300, at 10 mg/kg, was tested in behavioural assays, and compared to littermate controls. Locomotor, stereotypic movements and vertical activity were tested, along with anxiety/mania and sensorimotor gating behaviours. All data were analyzed with two- or three-way ANOVA, as appropriate, and corrected for multiple comparisons.

### Locomotor Activity, Stereotypy, and Vertical Activity (Open Field Test, OFT):

Classically, locomotor output has been used as a gold standard for the measure of dopamine system dysfunction. The role of dopamine in movement control is well defined and, in its simplest forms, testing for hyperactivity is based on the premise that enhanced dopaminergic activity in rodents leads to enhanced motor activity (van den Buuse et al., 2010). Locomotor, stereotypic and vertical activity was recorded using digital activity monitors, quantifying horizontal and vertical activity, along with repetitive behaviour, via infrared beam breaks. GluN1 KD and DATKO display basal hyperactivity, with increases in stereotypic behaviours which can be ameliorated with antipsychotics (Peleg-Raibstein et al., 2008).

### Sensorimotor Gating—Pre-Pulse Inhibition (PPI) of Acoustic Startle Response (ASR):

Loss of PPI is a common symptom found in SCZ and is widely accepted as an endophenotype. It is commonly considered an “interface” of psychosis and cognition. PPI deficits have been described in other psychiatric diseases; during acute mania in BD (Perry et al., 2001). PPI was measured via automated startle boxes. GluN1 KD and DATKO mice all display deficits in PPI, and this behaviour is responsive to treatment with antipsychotics.

### Non-Alcoholic Fatty Liver Disease (NAFLD) Model:

Diet-induced obese (DIO) mouse models can recapitulate the endocrine and metabolic dysfunction observed in obese humans, and thus serve as ideal preclinical models fortesting novel therapeutic compounds. To induce fatty liver or NAFL, male C57BL/6 mice (n=10 vehicle; n=10 CB1 NAM) are fed a high fat diet (60% calories from fat, TD.06414) for a minimum of 8 weeks starting at 6 weeks of age. Control mice (n=10; vehicle TD.08806) are fed a diet consisting of 10% calories from fat. Mice are weighed weekly to monitor weight gain, and food consumption assessed as the average amount of food consumed per day to determine a potential central effect on appetite suppression. After 8 weeks of HFD, mice will exhibit impaired glucose tolerance as assessed by glucose tolerance tests, and intervention with the CB1 NAM will commence. Mice are dosed daily with 5 or 10 mg/kg indole-oxadiazole or vehicle (5% ethanol: 5% Tween 80:90% saline) by i.p. administration. After 28 days of administration, glucose tolerance and insulin tolerance tests are conducted on separate days. Blood is collected by terminal cardiac puncture under isoflurane anesthetic followed by cervical dislocation to collect sufficient volumes for the subsequent measurement of multiple analytes (insulin, ALT, triglycerides, adiponectin, leptin). Liver is weighed and collected in formalin (H&E staining for histopathology) and OCT (Oil Red O staining for lipid) or snap frozen for mRNA, protein and the assessment of hepatic triglycerides. Gene expression analyses is conducted to assess the levels of CB1 receptor, FAAH, and various lipogenic and fatty acid transport genes.

### Human and Rat Liver Microsomal Stability Assay

Metabolic stability of indole-oxadiazole derivatives of Formula I was measured by determination of the rate of compound disappearance when incubated in the presence of human or rat liver microsomes. Liver microsomes are prepared from the endoplasmic reticulum of hepatocytes and are the primary source of the most important enzymes (cytochrome P450) involved in drug metabolism. Study of drug stability in the presence of liver microsomes is accepted as a valuable model permitting rapid prediction of in vivo drug stability.

### Protocol Summary:

Human and rat liver microsomes were obtained from commercial sources. Test compounds (3 μM) were incubated with pooled liver microsomes (male and female). Samples were incubated for a 45 minute period and removed at 5 time points and test compounds were analysed by LC-MS/MS.

Microsomes (final protein concentration 0.5 mg/mL), 0.1 M phosphate buffer pH 7.4, and test compound (final concentration 3 μM; diluted from 10 mM stock solution to give a final DMSO concentration of 0.25%) were incubated at 37° C. prior to the addition of NADPH (final concentration 1 mM) to initiate the reaction. The final incubation volume was 25 μL. A control incubation was included for each compound tested, where 0.1 M phosphate buffer pH 7.4 was added instead of NADPH. The control compounds testosterone and 7-hydroxycoumarin were included in each experiment and all incubations were performed singularly for each compound.

Each compound was incubated for 0, 5, 15, 30, and 45 minutes. The control (minus NADPH) was incubated for 45 minutes only. The reactions were stopped by the addition of 50 μL methanol containing internal standard at the appropriate time points. The incubation plates were centrifuged at 2500 rpm for 20 minutes at 4° C. to precipitate the protein.

### Quantitative Analysis:

Following protein precipitation, the sample supernatants were combined in cassettes of up to 4 compounds and analysed using standard LC-MS/MS conditions.

### Data Analysis:

From a plot of the natural logarithm of the peak area ratio (i.e., the ratio of compound peak area:internal standard peak area) against time, the gradient of the line was determined. Subsequently, half-life and intrinsic clearance were calculated using the equations below:

Eliminated rate constant (k)=(−gradient).

Half life (t_{1/2}) (min)=0.063/k.

Intrinsic Clearance (CLᵢₙₜ)(μL/min/million cells)=(V×0.693)/t_{1/2}.

wherein V=Incubation volume (μL/mg microsomal protein).

### Biological Study 1

Initial screening of candidate compounds was performed using an in vitro assay to determine functional characteristics.

The PathHunter™ β-Arrestin assay (from DiscoveRX, Fremont, USA) was performed as follows. HEK293 CB1 β-arrestin cells were plated 48 hours before use and incubated at 37° C., 5% CO₂ in a humidified incubator. Test compounds were dissolved in dimethylsulfoxide (DMSO) and diluted in optimized cell culture (OCC, as supplied by DiscoveRX) media to the required concentrations. 5 μL of test compound or vehicle solution was added to each well and incubated for 60 minutes at 37° C., 5% CO₂ in a humidified incubator. 5 μL of increasing concentrations of anandamide was added to each well followed by a 90-minute incubation at 37° C., 5% CO₂ in a humidified incubator. 55 μL of detection reagent (as supplied by DiscoveRX) was then added, followed by a further 90 minute incubation at room temperature in the dark. Chemiluminescence, reported in relative light units (RLU—a dimensionless value, standardised as a % of maximum stimulation with anandamide), was measured on a standard luminescence plate reader.

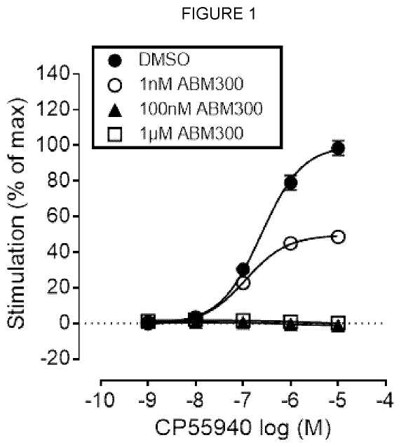

Data were plotted as % of maximal stimulation (Eₘₐₓ) caused by agonist versus the logarithm of concentration of agonist, in the presence or absence of a fixed concentration of test compound (modulator) or vehicle. For example, stimulation in the presence of agonist alone will give a value for stimulation expressed in RLU, which can be expressed as a range from 0% (at lowest agonist concentration, e.g., 1 nM) to 100% (at the highest agonist concentration, e.g., 10 μM). Addition of a test compound which is a negative allosteric modulator at a fixed concentration (e.g., 100 nM) will give reduced RLU values leading to a reduction in the maximum stimulation elicited by the agonist, which can be expressed as a % of the control (agonist alone). The concentration of modulator which produces a 50% reduction in the Eₘₐₓ of the agonist is defined as the IC₅₀. An example of this is shown in FIG. 1.

FIG. 1 is a graph illustrating the data described herein, and shows the effects of ABM300 in inhibiting the maximum level of stimulation (Eₘₐₓ) caused by the cannabinoid agonist CP55,940, as measured using the β-arrestin assay. The graph shows that ABM300 is a highly potent inhibitor of cannabinoid receptor activity and reduces the level of stimulation (Eₘₐₓ, efficacy) by 50% at a concentration of close to 1 nM (IC₅₀=1 nM, as calculated by GraphPad Prism). Each symbol represents the mean percentage of stimulation above basal ±S.E.M (n=3).

The graph also demonstrates that ABM300 is an allosteric inhibitor (negative allosteric modulator) because it gives a reduction in Eₘₐₓ which is not overcome by higher concentrations of agonist; this is in contrast to the rightward shift of the curve, without a reduction in Eₘₐₓ that would be seen with a competitive orthosteric antagonist.

All data were analysed using GraphPad Prism 5 and fitted to a sigmoidal dose response curve; the IC₅₀ value was obtained as the concentration of the inhibitor that reduces the agonist Eₘₐₓ to 50% of the maximum response obtained for the agonist.

The following compounds were studied using the PathHunter™ β-Arrestin assay as described above:

All of the compounds have an IC₅₀ for allosteric inhibition of less than 10 μM.

Data for several compounds of Formula I are shown below.

| Compound I.D. | Structure | IC₅₀ (nM) |
|---|---|---|
| ABM300 | 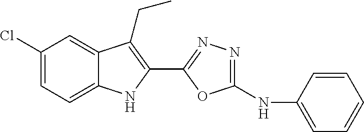 [SMILES: CCc1c(-c2nnc(Nc3ccccc3)o2)[nH]c2ccc(Cl)cc12] | 1 |
| ABM310 | 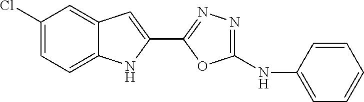 [SMILES: Clc1ccc2[nH]c(-c3nnc(Nc4ccccc4)o3)cc2c1] | 3 |
| ABM311 | 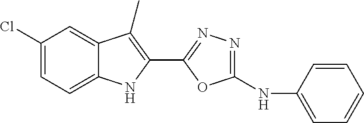 [SMILES: Cc1c(-c2nnc(Nc3ccccc3)o2)[nH]c2ccc(Cl)cc12] | 2 |
| ABM312 | 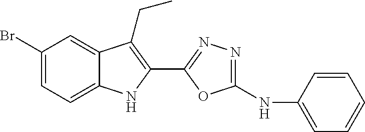 [SMILES: CCc1c(-c2nnc(Nc3ccccc3)o2)[nH]c2ccc(Br)cc12] | 1 |
| ABM313 | 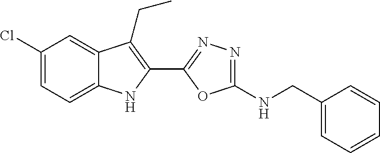 [SMILES: CCc1c(-c2nnc(NCc3ccccc3)o2)[nH]c2ccc(Cl)cc12] | 12 |
| ABM314 | 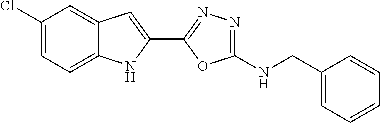 [SMILES: Clc1ccc2[nH]c(-c3nnc(NCc4ccccc4)o3)cc2c1] | 105 |
| ABM315 | 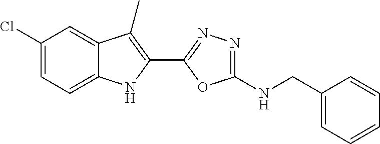 [SMILES: Cc1c(-c2nnc(NCc3ccccc3)o2)[nH]c2ccc(Cl)cc12] | 7 |
| ABM316 | 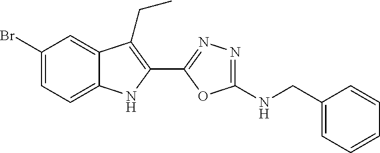 [SMILES: CCc1c(-c2nnc(NCc3ccccc3)o2)[nH]c2ccc(Br)cc12] | 12 |
| ABM317 | 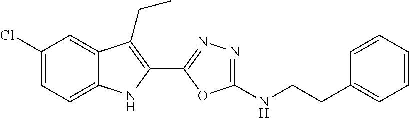 [SMILES: CCc1c(-c2nnc(NCCc3ccccc3)o2)[nH]c2ccc(Cl)cc12] | 0.5 |
| ABM318 | 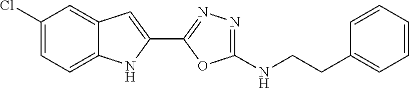 [SMILES: Clc1ccc2[nH]c(-c3nnc(NCCc4ccccc4)o3)cc2c1] | 3 |
| ABM319 | 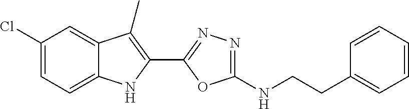 [SMILES: Cc1c(-c2nnc(NCCc3ccccc3)o2)[nH]c2ccc(Cl)cc12] | 1 |
| ABM320 | 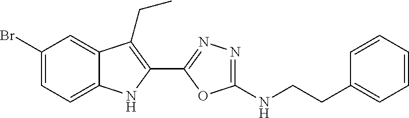 [SMILES: CCc1c(-c2nnc(NCCc3ccccc3)o2)[nH]c2ccc(Br)cc12] | 1 |
| ABM335 | 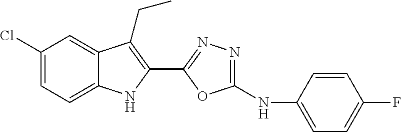 [SMILES: CCc1c(-c2nnc(Nc3ccc(F)cc3)o2)[nH]c2ccc(Cl)cc12] | 1 |
| ABM336 | 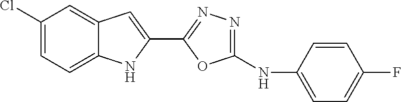 [SMILES: Fc1ccc(Nc2nnc(-c3cc4cc(Cl)ccc4[nH]3)o2)cc1] | 5 |
| ABM337 | 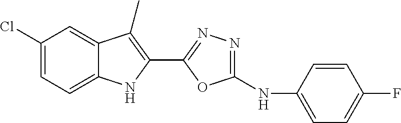 [SMILES: Cc1c(-c2nnc(Nc3ccc(F)cc3)o2)[nH]c2ccc(Cl)cc12] | 1 |
| ABM338 | 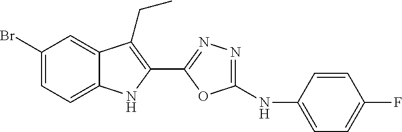 [SMILES: CCc1c(-c2nnc(Nc3ccc(F)cc3)o2)[nH]c2ccc(Br)cc12] | 1 |
| ABM301 | 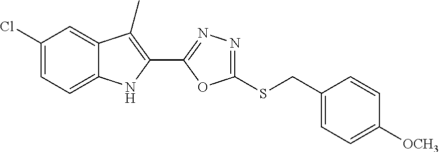 [SMILES: COc1ccc(CSc2nnc(-c3[nH]c4ccc(Cl)cc4c3C)o2)cc1] | 68 |
| ABM305 | 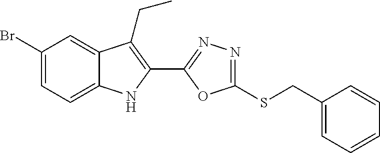 [SMILES: CCc1c(-c2nnc(SCc3ccccc3)o2)[nH]c2ccc(Br)cc12] | 30 |
| ABM321 | 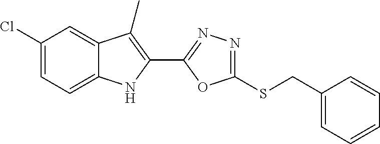 [SMILES: Cc1c(-c2nnc(SCc3ccccc3)o2)[nH]c2ccc(Cl)cc12] | 93 |
| ABM322 | 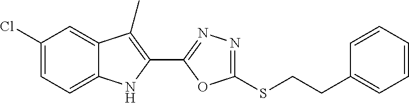 [SMILES: Cc1c(-c2nnc(SCCc3ccccc3)o2)[nH]c2ccc(Cl)cc12] | 417 |
| ABM323 | 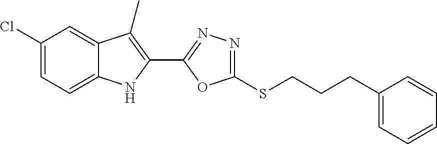 [SMILES: Cc1c(-c2nnc(SCCCc3ccccc3)o2)[nH]c2ccc(Cl)cc12] | 263 |
| ABM325 | 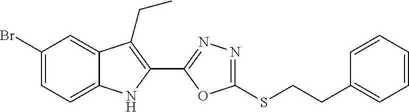 [SMILES: CCc1c(-c2nnc(SCCc3ccccc3)o2)[nH]c2ccc(Br)cc12] | 315 |
| ABM327 | 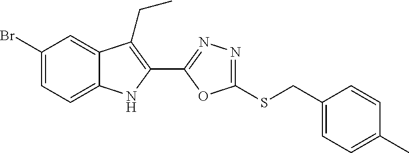 [SMILES: CCc1c(-c2nnc(SCc3ccc(C)cc3)o2)[nH]c2ccc(Br)cc12] | 67 |
| ABM328 | 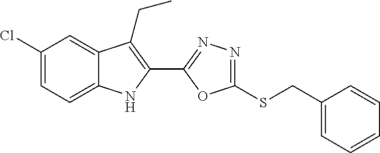 [SMILES: CCc1c(-c2nnc(SCc3ccccc3)o2)[nH]c2ccc(Cl)cc12] | 58 |
| ABM329 | 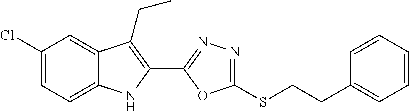 [SMILES: CCc1c(-c2nnc(SCCc3ccccc3)o2)[nH]c2ccc(Cl)cc12] | 930 |
| ABM331 | 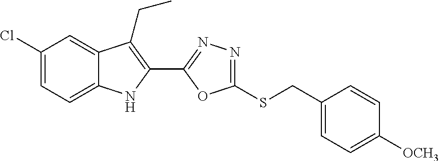 [SMILES: CCc1c(-c2nnc(SCc3ccc(OC)cc3)o2)[nH]c2ccc(Cl)cc12] | 56 |
| ABM332 | 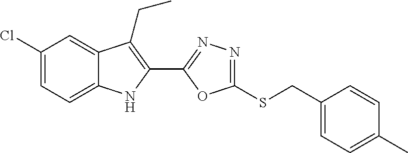 [SMILES: CCc1c(-c2nnc(SCc3ccc(C)cc3)o2)[nH]c2ccc(Cl)cc12] | 85 |
| ABM334 | 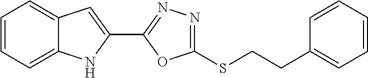 [SMILES: c1ccc(CCSc2nnc(-c3cc4ccccc4[nH]3)o2)cc1] | 172 |
| ABM339 | 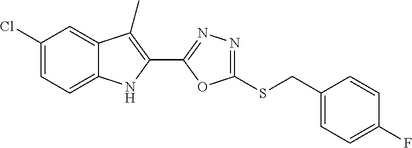 [SMILES: Cc1c(-c2nnc(SCc3ccc(F)cc3)o2)[nH]c2ccc(Cl)cc12] | 270 |
| ABM340 | 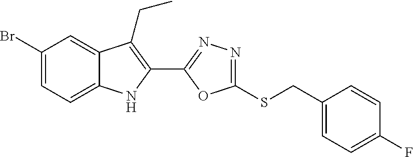 [SMILES: CCc1c(-c2nnc(SCc3ccc(F)cc3)o2)[nH]c2ccc(Br)cc12] | 19 |

Thus the data demonstrates that indole-oxadiazole compounds of the application are highly potent CB1 negative allosteric modulators.

### Biological Study 2

The metabolic stability of a number of indole-oxadiazole compounds of Formula I was determined and compared with the metabolic stability of a range of structurally related compounds using the assays described previously.

Biological half-life values (t_{1/2}) were determined for several indole-oxadiazole compounds, as well as several reference compounds, using the human and rat liver microsomal stability assay described above. The results are summarised in the following table.

Liver Microsomal Stability Data

| | | Microsomal | |
|---|---|---|---|
| | | stability | |
| | | T_{1/2} (min) | |
| Compound I.D. | Structure | Human | Rat |
| Reference compound 1 (Org27569) | 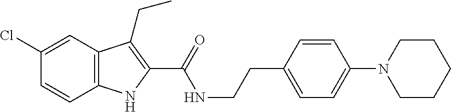 [SMILES: CCc1c(C(=O)NCCc2ccc(N3CCCCC3)cc2)[nH]c2ccc(Cl)cc12] | 32 | 28 |
| Reference compound 2 (ABD1075) | 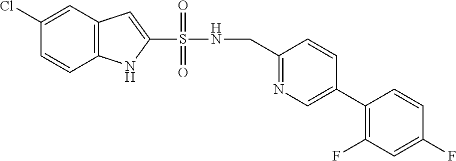 [SMILES: O=S(=O)(NCc1ccc(-c2ccc(F)cc2F)cn1)c1cc2cc(Cl)ccc2[nH]1] | 56 | 32 |
| Reference compound 3 (ABD1085) | 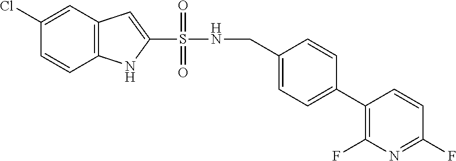 [SMILES: O=S(=O)(NCc1ccc(-c2ccc(F)nc2F)cc1)c1cc2cc(Cl)ccc2[nH]1] | 39 | 48 |
| ABM300 | 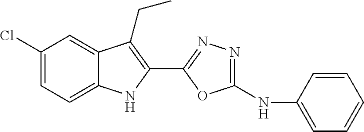 [SMILES: CCc1c(-c2nnc(Nc3ccccc3)o2)[nH]c2ccc(Cl)cc12] | 109 | 110 |
| ABM338 | 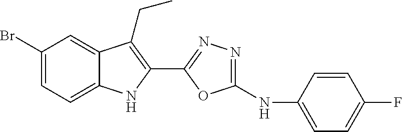 [SMILES: CCc1c(-c2nnc(Nc3ccc(F)cc3)o2)[nH]c2ccc(Br)cc12] | 174 | 292 |
| ABM328 | 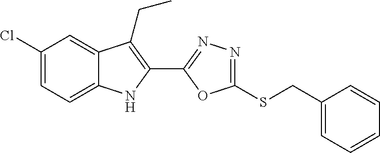 [SMILES: CCc1c(-c2nnc(SCc3ccccc3)o2)[nH]c2ccc(Cl)cc12] | 46 | 40 |
| ABM340 | 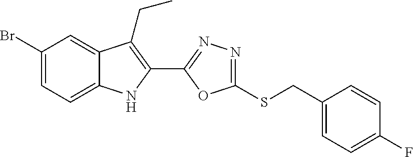 [SMILES: CCc1c(-c2nnc(SCc3ccc(F)cc3)o2)[nH]c2ccc(Br)cc12] | 40 | 23 |

These data demonstrate that it is possible to replace the amide group of reference compound 1, Org27569 (Price et al, 2005) or the sulfonamide group of reference compounds 2 and 3, ABD1075 and ABD1085 (Greig et al., 2016), with an oxadiazole with a considerable increase in metabolic stability. The data also demonstrate that this replacement is neither trivial nor predictable and can lead either to an increase or a decrease in metabolic stability.

Thus the data demonstrates that the indole-oxadiazole compounds of the application can be highly metabolically stable and thus have potential as therapeutic agents.

### Biological Study 3—Behavioral Deficits in Animal Models of Schizophrenia and Bipolar Disorder.

ABM300 (10 mg/kg) reduces hyperactivity in GluN1KD mice and DATKO mice. It also reduces abnormal stereotypic behaviours measured in open field (OF) test in these mice. In GluN1KD mice and DATKO mice ABM300 (10 mg/kg) reduces abnormal vertical exploration (risk-taking behaviour). Total vertical activity was measured using the open field (OF) test. ABM300 (10 mg/kg) rescues sensorimotor gating deficits in DATKO mice.

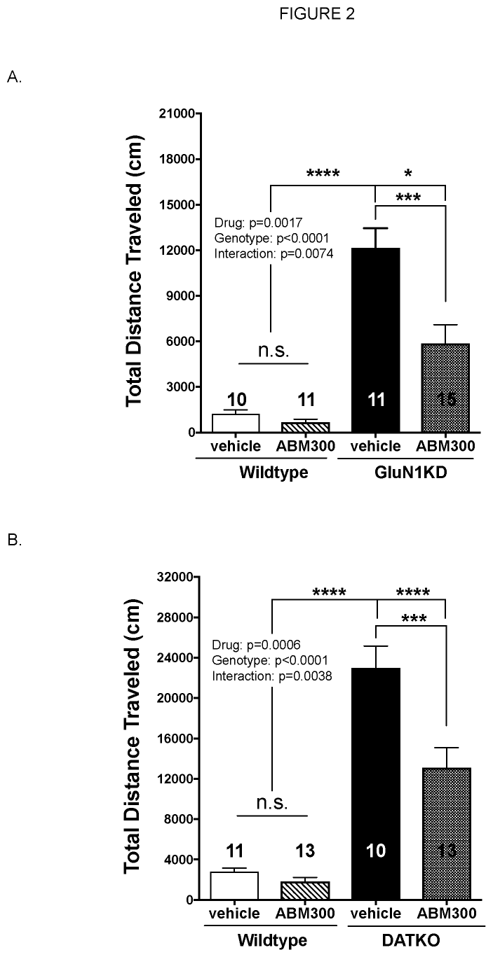

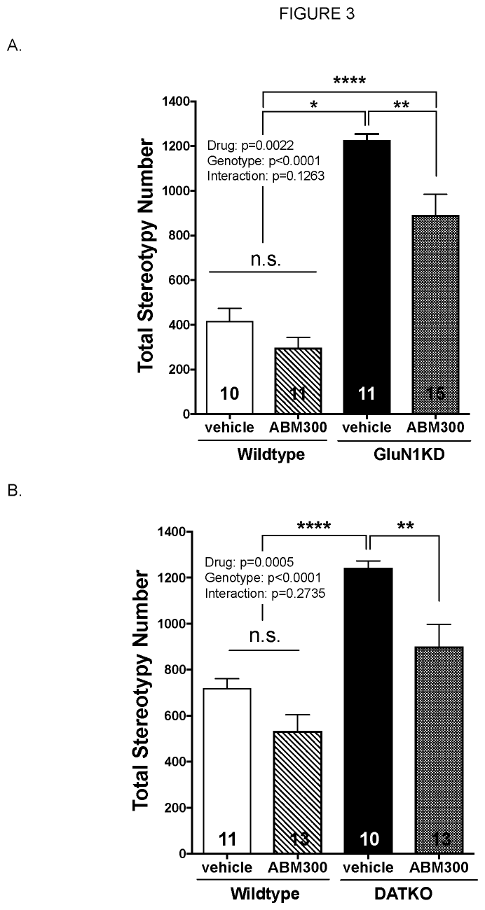

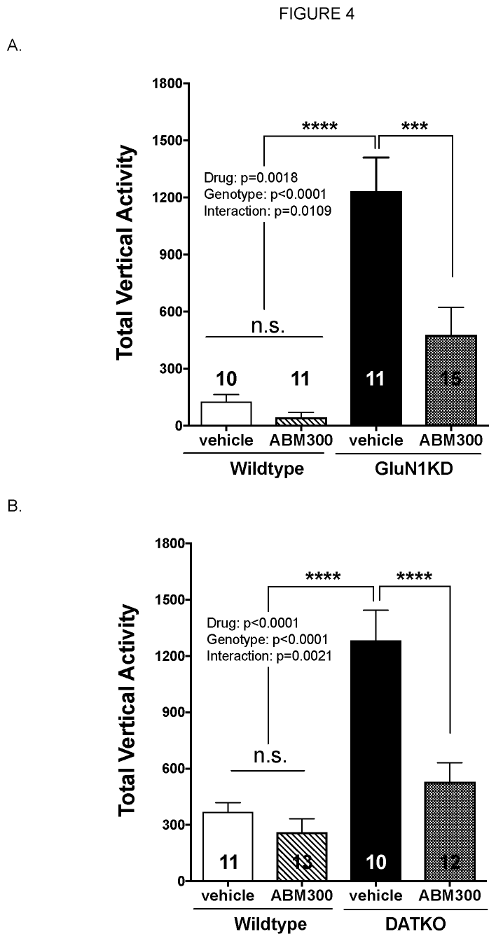

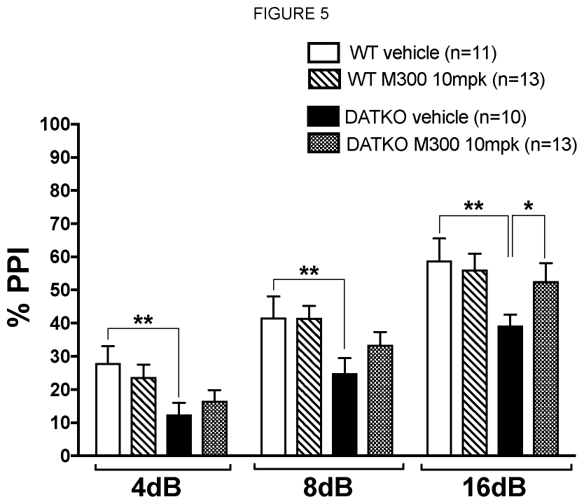

FIGS. 2, 3, 4 and 5 are graphs which show the beneficial effects of ABM300 in a range of mouse models that display behaviour deficit models indicative of dopamine dysregulation.

The results are summarised in the following table

| | WT | | GluN1KD | |
|---|---|---|---|---|
| Behaviour | | 10 mg/kg | | 10 mg/kg |
| Experiment | vehicle | ABM300 | vehicle | ABM300 |
| Locomotor Activity | 1229 ± 255 | 688 ± 193 | 12157 ± 1294 | 5857 ± 1242*** |
| Stereotypy | 417 ± 56.07 | 298.80 ± 45 | 1227 ± 27 | 891 ± 93** |
| Vertical Activity | 127 ± 36 | 43.91 ± 26 | 1233 ± 176 | 479 ± 142*** |

| | WT | | DATKO | |
|---|---|---|---|---|
| Behaviour | | 10 mg/kg | | 10 mg/kg |
| Experiment | vehicle | ABM300 | vehicle | ABM300 |
| Locomotor Activity | 2815 ± 351 | 1851 ± 378 | 22992 ± 2157 | 13120 ± 1961*** |
| Stereotypy | 720 ± 40 | 533 ± 69 | 1243 ± 30 | 900 ± 96** |
| Vertical Activity | 370 ± 48 | 262 ± 69 | 1285 ± 159 | 530 ± 101**** |

Thus, indole-oxadiazoles of Formula I show potential for the treatment of certain aspects of schizophrenia and bipolar disorder.

### Biological Study 4—Non Alcoholic Fatty Liver Disease.

ABM300 significantly reduces the levels of triglycerides and serum ALT, both markers for liver disease, in a mouse model of fatty liver disease. It also reduces the macrovesicular and microvesicular steatosis observed in mice with fatty liver disease.

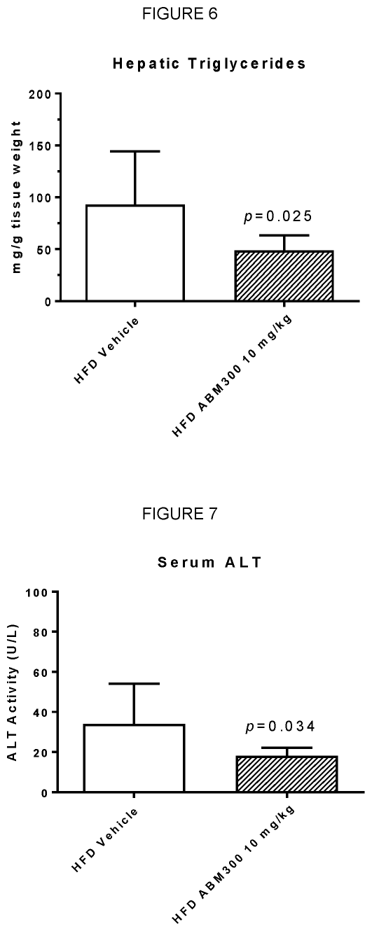

FIGS. 6 and 7 are graphs which show the beneficial effects of ABM300 in a mouse model for fatty liver disease.

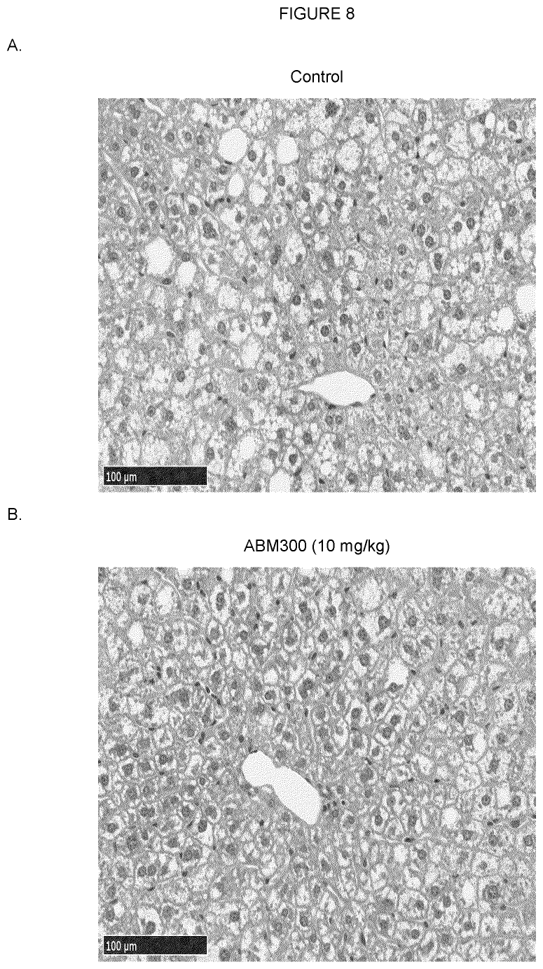

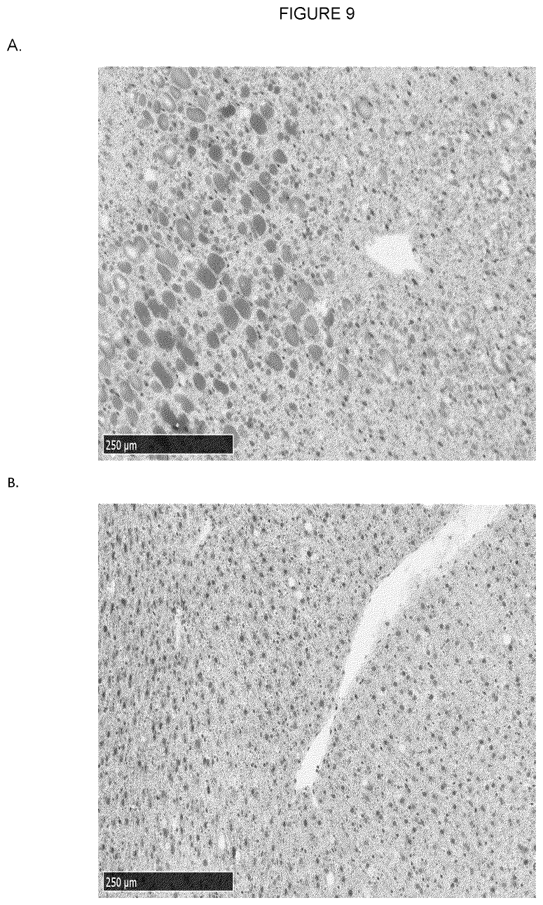

FIGS. 8 and 9 are representative images which show the beneficial effects of ABM300 in a mouse model for fatty liver disease.

The results are summarised in the following table

| | |
|---|---|
| Hepatic Triglycerides | Serum ALT |
| (mg/g tissue weight) | (ALT activity (U/L) |

| | ABM300 | | ABM300 |
|---|---|---|---|
| Vehicle | (10 mg/kg) | Vehicle | (10 mg/kg) |
| 93.1 ± 51.2 | 48.9 ± 14.6 | 34.0 ± 20.1 | 18.1 ± 4.0 |

Thus, indole-oxadiazoles of Formula I show potential for the treatment of liver disorders, including non-alcoholic fatty liver disease.

Thus, indole-oxadiazoles of Formula I show potential for the treatment of liver disorders, including those induced by anti-psychotics.

Thus, indole-oxadiazoles of Formula I show potential for the treatment of liver disorders, including those found in patients with schizophrenia and bipolar disorder.

The foregoing has described the principles, preferred embodiments, and modes of operation of the present application. However, the application should not be construed as limited to the particular embodiments discussed. Instead, the above-described embodiments should be regarded as illustrative rather than restrictive, and it should be appreciated that variations may be made in those embodiments by workers skilled in the art without departing from the scope of the present application.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### FULL CITATIONS FOR DOCUMENTS REFERRED TO IN THE SPECIFICATION

A number of publications are cited herein. Full citations for these references are provided below. Each of these references is incorporated herein by reference in its entirety into the present disclosure, to the same extent as if each individual reference was specifically and individually indicated to be incorporated by reference.
Arias et al., 2017, “Bipolar disorder and substance use disorders. Madrid study on the prevalence of dual disorders/pathology,” Adicciones, Vol. 29, pp. 186-194.
Baillie et al., 2013, “CB1 receptor allosteric modulators display both agonist and activity pathway specificity,” Mol. Pharmacol., Vol. 83, pp 322-38.
Beaulieu, J.-M. et al. 2004, “Lithium antagonizes dopamine-dependent behaviors mediated by an AKT/glycogen synthase kinase 3 activity cascade,” Proc. Natl. Acad. Sci. U.S.A, Vol. 101, pp. 5099-104
Bermúdez-Silva et al., 2008, “Presence of functional cannabinoid receptors in human endocrine pancreas,” Diabetologia, Vol 51 (3), pp 476-87.
Christopoulos et al., 2002, “G protein-coupled receptor allosterism and complexing,” Pharmacol. Rev., Vol. 54, pp. 323-374.
Christopoulos et al., 2004, “G-protein-coupled receptor allosterism: the promise and the problem(s),” Biochemical Society Transactions, Vol. 32, pp. 873-877.
Conn et al., 2009, “Allosteric modulators of GPCRs: a novel approach for the treatment of CNS disorders,” Nat. Rev. Drug Disc., Vol. 8, pp. 41-54
Cota et al., 2003, “The endogenous cannabinoid system affects energy balance via central orexigenic drive and peripheral lipogenesis,” J. Clin. Invest., Vol. 112, pp. 423-431.
Covey et al., 2017, “Endocannabinoid modulation of dopamine neurotransmission. Neuropharmacology,” Vol. 124, pp. 52-61
Croci et al., 2003, “Role of cannabinoid CB1 receptors and tumor necrosis factor-alpha in the gut and systemic anti-inflammatory activity of SR 141716 (rimonabant) in rodents,” Brit. J. Pharmacol., Vol. 140, pp. 115-122.
Demontis et al., 2011, “Association of GRIN1 and GRIN2A-D with schizophrenia and genetic interaction with maternal herpes simplex virus-2 infection affecting disease risk,” Am. J. Med. Genet. B. Neuropsychiatr. Genet., Vol. 156B, pp. 913-22
Dietrich and Hellerbrand, 2014 “Non-alcoholic fatty liver disease, obesity and the metabolic syndrome,” Best Pract Res Clin Gastroenterol, Vol. 28, pp. 637-653.
Di Marzo et al., 2004, “The endocannabinoid system and its therapeutic exploitation,” Nat. Rev. Drug Disc., Vol. 3, pp. 771-784.
Di Marzo 2008, “Targeting the endocannabinoid system: to enhance or reduce?” Nat. Rev. Drug Disc., Vol. 7, pp. 438-455.
Dopart et al., 2018, “Allosteric modulators of cannabinoid receptor 1: developing compounds for improved specificity,” Drug Metab. Rev., Vol. 50, pp. 3-13.
El Ashry et al., 2013, “Immunomodulatory properties of S- and N-alkylated 5-(1H-indol-2-yl)-1,3,4-oxadiazole-2(3H)-thione,” Journal of Enzyme Inhibition and Medicinal Chemistry, Vol. 28, pp. 105-112.
Ferris et al. 2014, “Sustained N-methyl-D-aspartate receptor hypofunction remodels the dopamine system and impairs phasic activity,” Eur. J. Neurosci. Vol. 40, pp. 2255-63.
French et al., 2015, “Early Cannabis Use, Polygenic Risk Score for Schizophrenia and Brain Maturation in Adolescence. JAMA Psychiatry, Vol. 72, pp. 1002-11.
Gainetdinov et al., 1999, “Functional hyperdopaminergia in dopamine transporter knock-out mice,” Biol. Psychiatry, Vol. 46, pp. 303-11.
Gao et al., 2006, “Keynote review: Allosterism in membrane receptors,” Druq Disc. Today, Vol. 11, pp. 191-202.
Greig et al., 2004, international patent application publication number WO 2004/078261 A1 published 16 Sep. 2004.
Greig et al., 2016, “Development of indole sulfonamides as cannabinoid receptor negative allosteric modulators,” Bioorg. Med. Chem. Lett., Vol. 26, pp. 4403-4407
Hiremath et al., 1989, “Synthesis of oxadiazolyl-, thiadiazolyl-, and triazolylindoles and indolylthiazolidinones,” Indian Journal of Chemistry, Section B: Organic Chemistry Including Medicinal Chemistry, Vol. 28B(8), pp. 626-30.
Hurst et al., 2009, “Preparation of oxadiazole derivatives as nicotinic acetylcholine receptor modulators,” international patent application publication number WO 2009071577.
Idris et al., 2005, “Regulation of bone mass, bone loss and osteoclast function by cannabinoid receptors”, Nat. Med., Vol. 11, pp. 774-779.
Jeong et al., 2008, “Paracrine activation of hepatic CB1 receptors by stellate cell-derived endocannabinoids mediates alcoholic fatty liver.” Cell Metab., Vol. 7, pp. 227-235.
Kunos et al., 2008, “Endocannabinoids and the control of energy homeostasis.” J Biol. Chem., Vol. 283, pp. 33021-33025.
Kunos et al., 2009, “Should peripheral CB(1) cannabinoid receptors be selectively targeted for therapeutic gain?” Trends Pharmacol. Sci., Vol. 30, pp. 1-7.
Martins et al., 2014, “The endocannabinoid system—back to the scene of cardiometabolic risk factors control?” Horm Metab Res, Vol. 46, pp. 529-536.
May et al., 2003, “Allosteric modulators of G-protein-coupled receptors,” Curr. Opin. Pharmacol., Vol. 3, pp. 551-556.
Mohn et al., 1999, “Mice with reduced NMDA receptor expression display behaviors related to schizophrenia.” Cell, Vol. 98, pp. 427-436.
Monge et al., 1992, “Synthesis and platelet aggregation inhibiting activity of new 1,3,4-oxadiazoles prepared by cyclodesulfurization of thiosemicarbazides with dicyclohexylcarbodiimide,” Anales de Quimica, Vol. 88, pp. 607-9.
Osei-Hyiaman et al., 2005, “Endocannabinoid activation at hepatic CB1 receptors stimulates fatty acid synthesis and contributes to diet-induced obesity,” J. Clin. Invest., Vol. 115, pp. 1298-1305.
Osei-Hyiaman et al., 2008, “Hepatic CB1 receptor is required for development of diet-induced steatosis, dyslipidemia, and insulin and leptin resistance in mice.” J. Clin. Invest., Vol. 118, pp. 3160-3169.
Peleg-Raibstein et al., 2008, “Amphetamine sensitization in rats as an animal model of schizophrenia,” Behav. Brain Res. Vol. 191, pp. 190-201.
Pertwee, 2005, “The Therapeutic potential of drugs that target cannabinoid receptors or modulate the tissue levels or actions of endocannabinoids,” AAPS Journal, Vol. 7, pp. E625-E654.
Pertwee, 2007, “Cannabinoids and multiple sclerosis,” Mol. Neurobiol., Vol. 36, pp. 45-59.
Perry et al., 2001, “Sensorimotor gating deficits in bipolar disorder patients with acute psychotic mania,” Biol. Psychiatry, Vol. 50, pp. 418-24.
Peters et al., 2004 “Preparation of novel 1,4-diazabicycloalkane derivatives as cholinergic ligands at the nicotinic acetylcholine receptors and modulators of the monoamine receptors and transporters,” international patent application publication number WO 2004029053.
Price et al., 2005, “Allosteric Modulation of the Cannabinoid CB1 Receptor, Mol. Pharmacol., Vol. 68, pp. 1484-1495.
Purohit et al., 2010, “Role of cannabinoids in the development of fatty liver (steatosis).” AAPS J 12(2): 233-237.
Purves-Tyson et al., 2017, “Putative presynaptic dopamine dysregulation in schizophrenia is supported by molecular evidence from post-mortem human midbrain. Transl. Psychiatry, Vol. 7, pp. e1003.
Quarta et al., 2011, “Energy balance regulation by endocannabinoids at central and peripheral levels.” Trends Mol. Med., Vol. 17, pp. 518-526.
Ralph et al., 2001, “Prepulse inhibition deficits and perseverative motor patterns in dopamine transporter knock-out mice: differential effects of D1 and D2 receptor antagonists,” J. Neurosci., Vol. 21, pp. 305-13.
Ravinet et al., 2004, “CB1 cannabinoid receptor knockout in mice leads to leanness, resistance to diet-induced obesity and enhanced leptin sensitivity.” Int. J. Obes. Relat. Metab. Disord., Vol. 28, pp. 640-648.
Rodriguez-MuAoz, et al., 2016, “Endocannabinoid control of glutamate NMDA receptors: the therapeutic potential and consequences of dysfunction,” Oncotarget, Vol. 7, pp. 55840-55862.
Rodriguez-MuAoz, et al., 2017, “Schizophrenia and depression, two poles of endocannabinoid system deregulation,” Transl. Psychiatry, 7, 1291.
Ross, 2003, “Anandamide and Vanilloid TRPV1 Receptors,” Br. J. Pharmacol., Vol 140, pp. 790-801.
Ross, 2007, “Allosterism and cannabinoid CB(1) receptors: the shape of things to come,” Trends Pharmacol. Sci., Vol. 28, pp. 567-572.
Ryberg et al., 2007, “The orphan receptor GPR55 is a novel cannabinoid receptor,” Br. J. Pharmacol., Vol. 152, pp. 1092-1101.
Scheen et al., 2006, “Efficacy and tolerability of rimonabant in overweight or obese patients with type 2 diabetes: a randomised controlled study,” Lancet, Vol. 368, pp. 1660-1672.
Simon and Cota, 2017, “Mechanisms in Endocrinology: Endocannabinoids and metabolism: past, present and future.” Eur. J. Endocrinol., Vol. 176, pp. R309-R324
Son et al., 2010, “Peripherally acting CB1-receptor antagonist: the relative importance of central and peripheral CB1 receptors in adiposity control,” Int. J. Obesity, Vol. 34, pp. 547-556.
Tam et al., 2010, “Peripheral CB1 cannabinoid receptor blockade improves cardiometabolic risk in mouse models of obesity,” J. Clin. Invest., Vol. 120, pp. 2953-2966.
Tourino et al., 2010, “FAAH deficiency promotes energy storage and enhances the motivation for food.” Int. J. Obes. (Lond.), Vol. 34, pp. 557-568. van den Buuse et al., 2010, “Modeling the positive symptoms of schizophrenia in genetically modified mice: pharmacology and methodology aspects,” Schizophr. Bull., Vol. 36, pp. 246-70.
Van Gaal et al., 2005, “Effects of the cannabinoid-1 receptor blocker rimonabant on weight reduction and cardiovascular risk factors in overweight patients: 1-year experience from the RIO-Europe study,” Lancet, Vol. 365, pp. 1389-1397.
Violin et al., 2007, “Beta-arrestin-biased ligands at seven-transmembrane receptors,” Trends Pharmacol. Sci., Vol. 28, pp. 416-422.
Vasu et al., 2014, “Synthesis and screening of some novel indole based 5-sulfanyl-[1,3,4]-oxadiazole,” Journal of Applicable Chemistry (Lumami, India), Vol. 3, Vol. 572-576.
Vettor and Pagano, 2009, “The role of the endocannabinoid system in lipogenesis and fatty acid metabolism,” Best Pract Res Clin Endocrinol Metab, Vol. 23, pp. 51-63.
Wang et al., 2009, “Allosteric modulators of G protein-coupled receptors: future therapeutics for complex physiological disorders,” J. Pharmacol. Exp. Ther., Vol. 331, pp. 340-348.
Wang et al., 2016, “Transition-Metal-Free Direct Arylation and Esterification Reaction of Unprotected Indolylcarboxylic Acid Derivatives: A New Entry to 2-(1H-Indol-2-yl)-5-(phenylthio)-1,3,4-oxadiazole and Aryl 1H-Indole-2-carboxylates,” Synlett., Vol. 27, pp. 2616-2620
Wang et al., 2017, “Theory of mind impairment and its clinical correlates in patients with schizophrenia, major depressive disorder and bipolar disorder. Schizophr. Res., doi:10.1016/j.schres.2017.11.003

1. A method for treating a disease, disorder or condition in a subject by modulating cannabinoid receptor activity comprising administering a therapeutically effective amount of one or more compounds of Formula I or a pharmaceutically acceptable salt and/or solvate thereof: 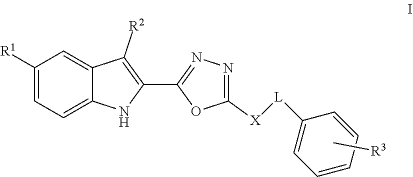
wherein:
R¹ is H, Br, Cl, F, I, C₁₋₆alkyl, SC₁₋₆alkyl or OC₁₋₆alkyl;
R² is H or C₁₋₆alkyl;
L is C₀₋₃alkylene;
R³ is H, Br, Cl, F, I, C₁₋₆alkyl, SC₁₋₆alkyl or OC₁₋₆alkyl; and
X is independently NH or S;
wherein all alkyl and alkylene are optionally fluorosubstituted.

2. The method of claim 1, wherein R¹ is Cl or Br.

3. The method of claim 1, wherein R¹ is SCH₃ or CF₃.

4. The method of claim 1, wherein R² is C₁₋₆alkyl.

5. (canceled)

6. The method of claim 1, wherein R³ is CH₃ or CF₃

7. (canceled)

8. The method of claim 1, wherein R³ is SCH₃, OCH₃ or OCF₃.

9. The method of claim 1, wherein R³ is F.

10. The method of claim 1, wherein the compound of Formula I is selected from the compounds listed below:

| Compound | | |
|---|---|---|
| I.D. | Example # | Structure |
| ABM300 | 2 | 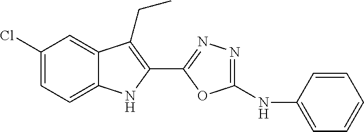 [SMILES: CCc1c(-c2nnc(Nc3ccccc3)o2)[nH]c2ccc(Cl)cc12] |
| ABM301 | 23 | 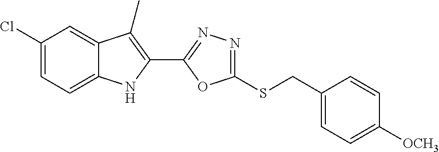 [SMILES: COc1ccc(CSc2nnc(-c3[nH]c4ccc(Cl)cc4c3C)o2)cc1] |
| ABM305 | 26 | 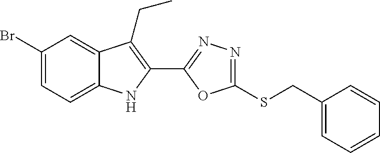 [SMILES: CCc1c(-c2nnc(SCc3ccccc3)o2)[nH]c2ccc(Br)cc12] |
| ABM309 | 27 | 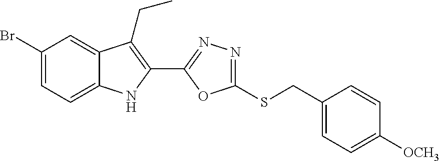 [SMILES: CCc1c(-c2nnc(SCc3ccc(OC)cc3)o2)[nH]c2ccc(Br)cc12] |
| ABM310 | 3 | 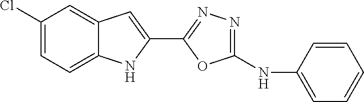 [SMILES: Clc1ccc2[nH]c(-c3nnc(Nc4ccccc4)o3)cc2c1] |
| ABM311 | 4 | 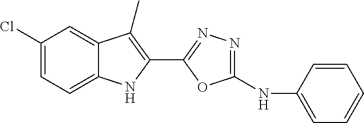 [SMILES: Cc1c(-c2nnc(Nc3ccccc3)o2)[nH]c2ccc(Cl)cc12] |
| ABM312 | 5 | 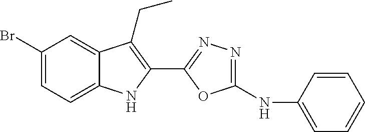 [SMILES: CCc1c(-c2nnc(Nc3ccccc3)o2)[nH]c2ccc(Br)cc12] |
| ABM313 | 7 | 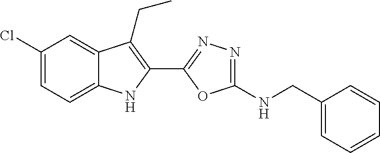 [SMILES: CCc1c(-c2nnc(NCc3ccccc3)o2)[nH]c2ccc(Cl)cc12] |
| ABM314 | 8 | 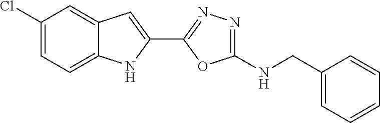 [SMILES: Clc1ccc2[nH]c(-c3nnc(NCc4ccccc4)o3)cc2c1] |
| ABM315 | 9 | 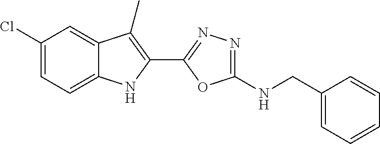 [SMILES: Cc1c(-c2nnc(NCc3ccccc3)o2)[nH]c2ccc(Cl)cc12] |
| ABM316 | 10 | 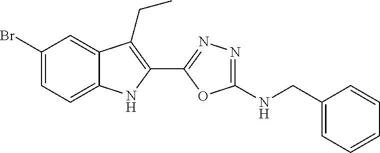 [SMILES: CCc1c(-c2nnc(NCc3ccccc3)o2)[nH]c2ccc(Br)cc12] |
| ABM317 | 12 | 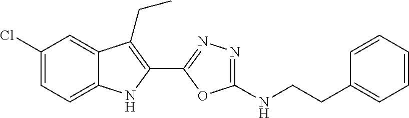 [SMILES: CCc1c(-c2nnc(NCCc3ccccc3)o2)[nH]c2ccc(Cl)cc12] |
| ABM318 | 13 | 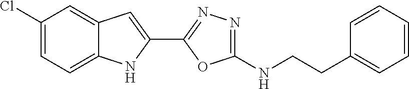 [SMILES: Clc1ccc2[nH]c(-c3nnc(NCCc4ccccc4)o3)cc2c1] |
| ABM319 | 14 | 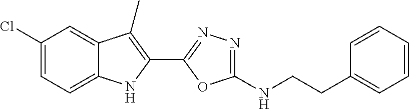 [SMILES: Cc1c(-c2nnc(NCCc3ccccc3)o2)[nH]c2ccc(Cl)cc12] |
| ABM320 | 15 | 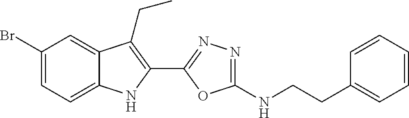 [SMILES: CCc1c(-c2nnc(NCCc3ccccc3)o2)[nH]c2ccc(Br)cc12] |
| ABM321 | 28 | 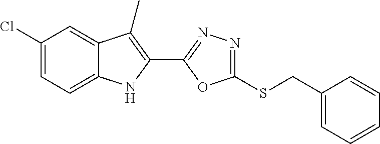 [SMILES: Cc1c(-c2nnc(SCc3ccccc3)o2)[nH]c2ccc(Cl)cc12] |
| ABM322 | 29 | 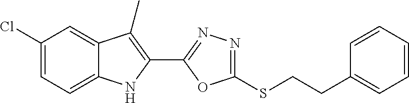 [SMILES: Cc1c(-c2nnc(SCCc3ccccc3)o2)[nH]c2ccc(Cl)cc12] |
| ABM323 | 30 | 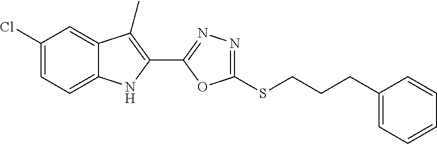 [SMILES: Cc1c(-c2nnc(SCCCc3ccccc3)o2)[nH]c2ccc(Cl)cc12] |
| ABM324 | 31 | 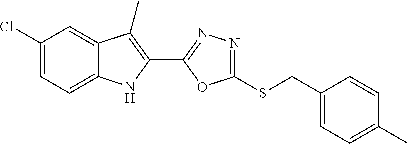 [SMILES: Cc1ccc(CSc2nnc(-c3[nH]c4ccc(Cl)cc4c3C)o2)cc1] |
| ABM325 | 32 | 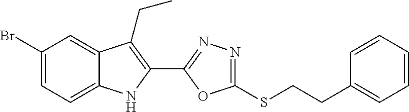 [SMILES: CCc1c(-c2nnc(SCCc3ccccc3)o2)[nH]c2ccc(Br)cc12] |
| ABM326 | 33 | 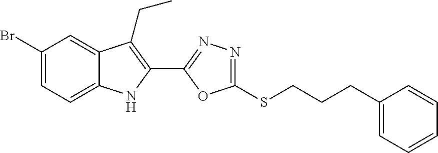 [SMILES: CCc1c(-c2nnc(SCCCc3ccccc3)o2)[nH]c2ccc(Br)cc12] |
| ABM327 | 34 | 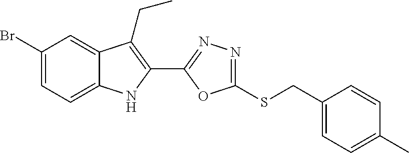 [SMILES: CCc1c(-c2nnc(SCc3ccc(C)cc3)o2)[nH]c2ccc(Br)cc12] |
| ABM328 | 37 | 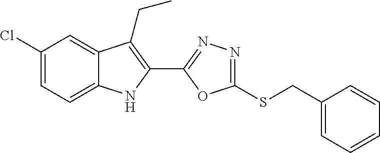 [SMILES: CCc1c(-c2nnc(SCc3ccccc3)o2)[nH]c2ccc(Cl)cc12] |
| ABM329 | 38 | 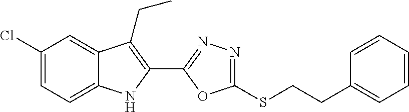 [SMILES: CCc1c(-c2nnc(SCCc3ccccc3)o2)[nH]c2ccc(Cl)cc12] |
| ABM330 | 39 | 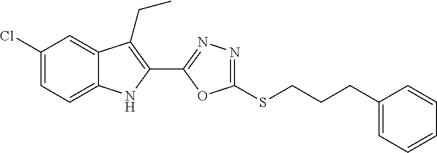 [SMILES: CCc1c(-c2nnc(SCCCc3ccccc3)o2)[nH]c2ccc(Cl)cc12] |
| ABM331 | 40 | 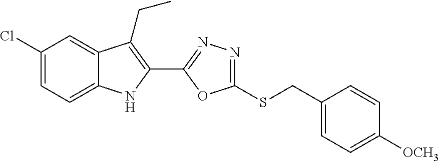 [SMILES: CCc1c(-c2nnc(SCc3ccc(OC)cc3)o2)[nH]c2ccc(Cl)cc12] |
| ABM332 | 41 | 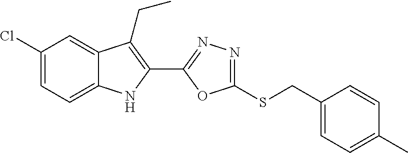 [SMILES: CCc1c(-c2nnc(SCc3ccc(C)cc3)o2)[nH]c2ccc(Cl)cc12] |
| ABM333 | 44 | 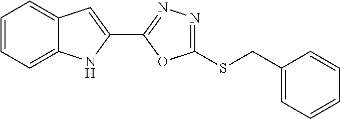 [SMILES: c1ccc(CSc2nnc(-c3cc4ccccc4[nH]3)o2)cc1] |
| ABM334 | 45 | 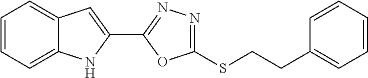 [SMILES: c1ccc(CCSc2nnc(-c3cc4ccccc4[nH]3)o2)cc1] |
| ABM335 | 17 | 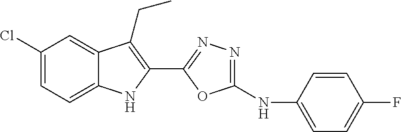 [SMILES: CCc1c(-c2nnc(Nc3ccc(F)cc3)o2)[nH]c2ccc(Cl)cc12] |
| ABM336 | 18 | 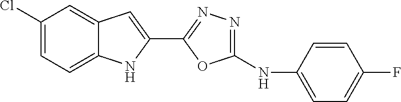 [SMILES: Fc1ccc(Nc2nnc(-c3cc4cc(Cl)ccc4[nH]3)o2)cc1] |
| ABM337 | 19 | 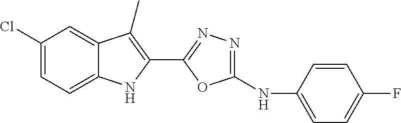 [SMILES: Cc1c(-c2nnc(Nc3ccc(F)cc3)o2)[nH]c2ccc(Cl)cc12] |
| ABM338 | 20 | 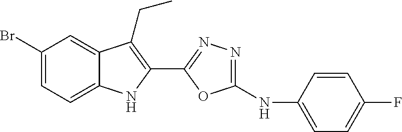 [SMILES: CCc1c(-c2nnc(Nc3ccc(F)cc3)o2)[nH]c2ccc(Br)cc12] |
| ABM339 | 46 | 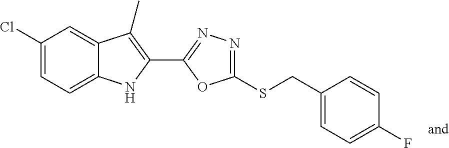 [SMILES: Cc1c(-c2nnc(SCc3ccc(F)cc3)o2)[nH]c2ccc(Cl)cc12] |
| ABM340 | 47 | 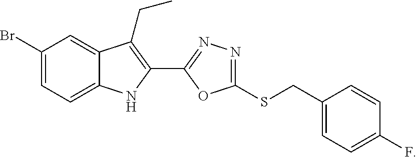 [SMILES: CCc1c(-c2nnc(SCc3ccc(F)cc3)o2)[nH]c2ccc(Br)cc12] |

or a pharmaceutically acceptable salt and/or solvate thereof.

11.-12. (canceled)

13. The method of claim 1, wherein the cannabinoid receptor is CB1.

14.-16. (canceled)

17. The method of claim 1, wherein the disease, disorder or condition that is treated by modulating cannabinoid receptor activity is a psychiatric disease, disorder or condition; a liver disease, disorder or condition; metabolic syndrome; type-2 diabetes; dyslipidaemia; obesity; eating disorder; cardiovascular disease or disease, disorder or condition associated with cardiovascular disease; a disease, disorder or condition characterised by an addiction component; a bone disease, disorder or condition; breast cancer; a disease, disorder or condition characterised by an inflammatory or an autoimmune component; and/or a disease, disorder or condition characterised by impairment of memory and/or loss of cognitive function.

18.-24. (canceled)

25. The method of claim 1, wherein the subject is a mammal.

26. (canceled)

27. A compound of Formula Ia or a pharmaceutically acceptable salt and/or solvate thereof: 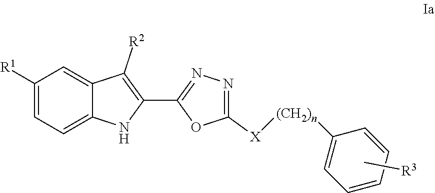
wherein:
R¹ is H, Br, Cl, F, I, C₁₋₆alkyl, SC₁₋₆alkyl or OC₁₋₆alkyl;
R² is H or C₁₋₆alkyl;
R³ is H, Br, Cl, F, I, C₁₋₆alkyl, SC₁₋₆alkyl or OC₁₋₆alkyl;
X is independently S or NH; and
n is 0, 1, 2 or 3;
wherein all alkyl are optionally fluorosubstituted,
provided
when X is S and n is 0 or 1 then R¹, R² and R³ are not all H;
when X is S, n is 1, R¹ is Br and R² is H then R³ is not H, CH₃ or Cl;
when X is NH, n is 0, R² is H and R³ is H then R¹ is not H, Br or Cl;
when X is NH, n is 0, R² is CH₃ and R³ is H then R¹ is not H or Cl;
when X is NH, n is 0, R¹ is CH₃ and R² is CH₃ then R³ is not H;
when X is NH, n is 0, R¹ is OCH₃ and R³ is H then R² is not H or CH₃;
when X is NH, n is 0, R¹ is OCH₂CH₃ and R³ is H then R² is not H.

28. The compound of claim 27, wherein R¹ is Cl or Br.

29. The compound of claim 27 wherein R¹ is SCH₃ or CF₃.

30. The compound of claim 27, wherein R² is C₁₋₆alkyl.

31. (canceled)

32. The compound of claim 27, wherein R³ is CH₃ or CF₃.

33. (canceled)

34. The compound of claim 27, wherein R³ is OCH₃ or OCF₃.

35. The compound of claim 27, wherein R³ is SCH₃.

36. The compound of claim 27, wherein R³ is F.

37. A compound selected from the compounds listed below:

| Compound | |
|---|---|
| I.D. | Structure |
| ABM300 | 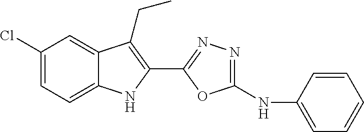 [SMILES: CCc1c(-c2nnc(Nc3ccccc3)o2)[nH]c2ccc(Cl)cc12] |
| ABM301 | 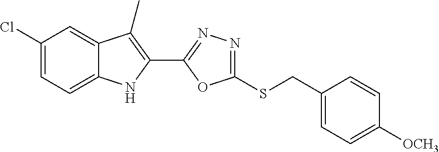 [SMILES: COc1ccc(CSc2nnc(-c3[nH]c4ccc(Cl)cc4c3C)o2)cc1] |
| ABM305 | 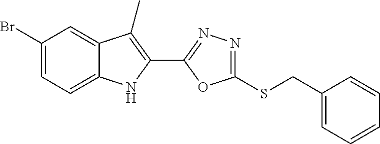 [SMILES: Cc1c(-c2nnc(SCc3ccccc3)o2)[nH]c2ccc(Br)cc12] |
| ABM309 | 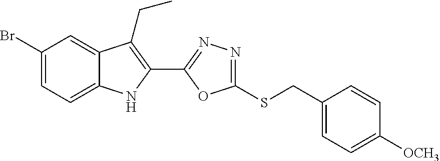 [SMILES: CCc1c(-c2nnc(SCc3ccc(OC)cc3)o2)[nH]c2ccc(Br)cc12] |
| ABM312 | 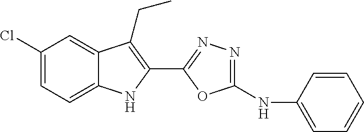 [SMILES: CCc1c(-c2nnc(Nc3ccccc3)o2)[nH]c2ccc(Cl)cc12] |
| ABM313 | 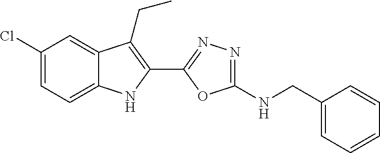 [SMILES: CCc1c(-c2nnc(NCc3ccccc3)o2)[nH]c2ccc(Cl)cc12] |
| ABM314 | 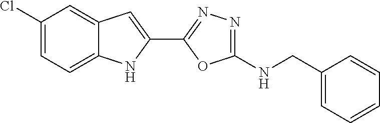 [SMILES: Clc1ccc2[nH]c(-c3nnc(NCc4ccccc4)o3)cc2c1] |
| ABM315 | 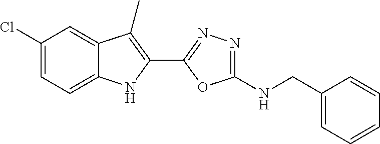 [SMILES: Cc1c(-c2nnc(NCc3ccccc3)o2)[nH]c2ccc(Cl)cc12] |
| ABM316 | 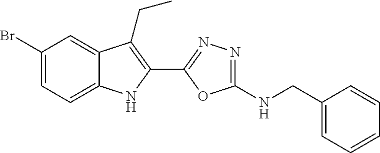 [SMILES: CCc1c(-c2nnc(NCc3ccccc3)o2)[nH]c2ccc(Br)cc12] |
| ABM317 | 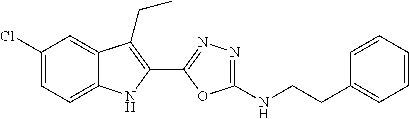 [SMILES: CCc1c(-c2nnc(NCCc3ccccc3)o2)[nH]c2ccc(Cl)cc12] |
| ABM318 | 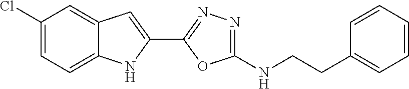 [SMILES: Clc1ccc2[nH]c(-c3nnc(NCCc4ccccc4)o3)cc2c1] |
| ABM319 | 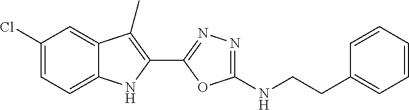 [SMILES: Cc1c(-c2nnc(NCCc3ccccc3)o2)[nH]c2ccc(Cl)cc12] |
| ABM320 | 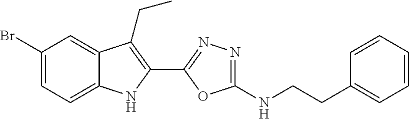 [SMILES: CCc1c(-c2nnc(NCCc3ccccc3)o2)[nH]c2ccc(Br)cc12] |
| ABM321 | 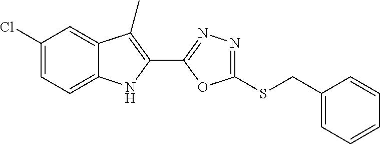 [SMILES: Cc1c(-c2nnc(SCc3ccccc3)o2)[nH]c2ccc(Cl)cc12] |
| ABM322 | 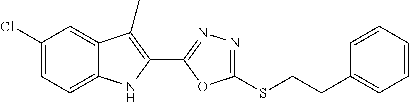 [SMILES: Cc1c(-c2nnc(SCCc3ccccc3)o2)[nH]c2ccc(Cl)cc12] |
| ABM323 | 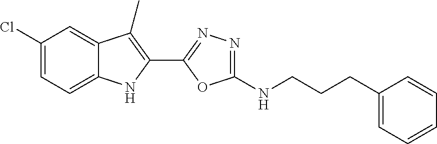 [SMILES: Cc1c(-c2nnc(NCCCc3ccccc3)o2)[nH]c2ccc(Cl)cc12] |
| ABM324 | 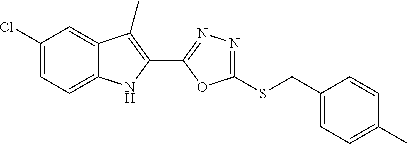 [SMILES: Cc1ccc(CSc2nnc(-c3[nH]c4ccc(Cl)cc4c3C)o2)cc1] |
| ABM325 | 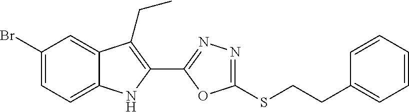 [SMILES: CCc1c(-c2nnc(SCCc3ccccc3)o2)[nH]c2ccc(Br)cc12] |
| ABM326 | 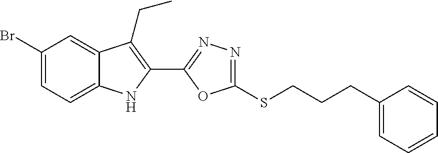 [SMILES: CCc1c(-c2nnc(SCCCc3ccccc3)o2)[nH]c2ccc(Br)cc12] |
| ABM327 | 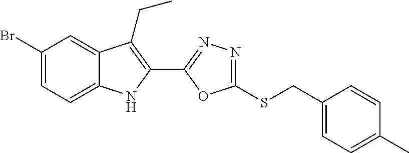 [SMILES: CCc1c(-c2nnc(SCc3ccc(C)cc3)o2)[nH]c2ccc(Br)cc12] |
| ABM328 | 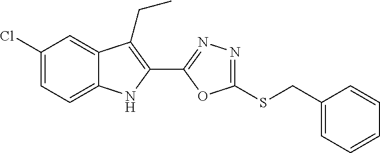 [SMILES: CCc1c(-c2nnc(SCc3ccccc3)o2)[nH]c2ccc(Cl)cc12] |
| ABM329 | 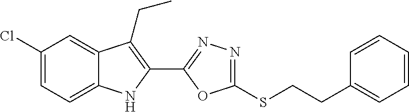 [SMILES: CCc1c(-c2nnc(SCCc3ccccc3)o2)[nH]c2ccc(Cl)cc12] |
| ABM330 | 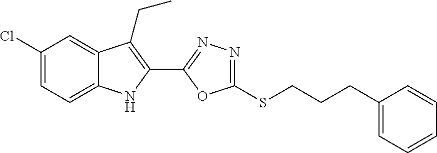 [SMILES: CCc1c(-c2nnc(SCCCc3ccccc3)o2)[nH]c2ccc(Cl)cc12] |
| ABM331 | 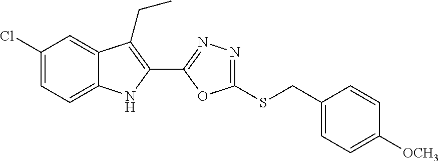 [SMILES: CCc1c(-c2nnc(SCc3ccc(OC)cc3)o2)[nH]c2ccc(Cl)cc12] |
| ABM332 | 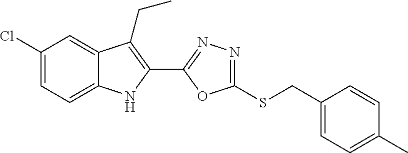 [SMILES: CCc1c(-c2nnc(SCc3ccc(C)cc3)o2)[nH]c2ccc(Cl)cc12] |
| ABM334 | 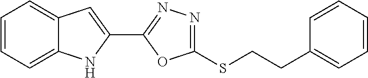 [SMILES: c1ccc(CCSc2nnc(-c3cc4ccccc4[nH]3)o2)cc1] |
| ABM335 | 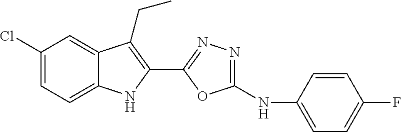 [SMILES: CCc1c(-c2nnc(Nc3ccc(F)cc3)o2)[nH]c2ccc(Cl)cc12] |
| ABM336 | 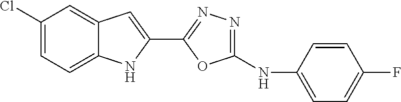 [SMILES: Fc1ccc(Nc2nnc(-c3cc4cc(Cl)ccc4[nH]3)o2)cc1] |
| ABM337 | 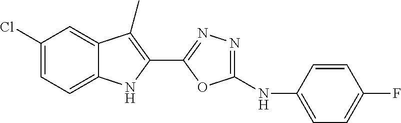 [SMILES: Cc1c(-c2nnc(Nc3ccc(F)cc3)o2)[nH]c2ccc(Cl)cc12] |
| ABM338 | 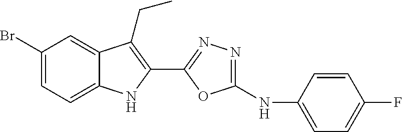 [SMILES: CCc1c(-c2nnc(Nc3ccc(F)cc3)o2)[nH]c2ccc(Br)cc12] |
| ABM339 | 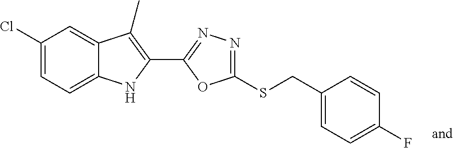 [SMILES: Cc1c(-c2nnc(SCc3ccc(F)cc3)o2)[nH]c2ccc(Cl)cc12] |
| ABM340 | 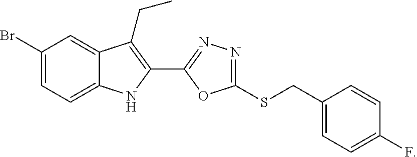 [SMILES: CCc1c(-c2nnc(SCc3ccc(F)cc3)o2)[nH]c2ccc(Br)cc12] |

or a pharmaceutically acceptable salt, and/or solvate thereof.

38.-39. (canceled)
